(19) United States
(12) Patent Application Publication
(10) Pub. No.: US 2021/0162051 A1
(43) Pub. Date: Jun. 3, 2021

(54) AQUEOUS-BASED COMPOSITIONS

(71) Applicants: Olufoyekemi BADEJO, Bridgewater, NJ (US); Reginald BRADLEY, Bedmister, NJ (US); Bayer HealthCare LLC, Whippany, NJ (US)

(72) Inventors: Olufoyekemi BADEJO, Bridgewater, NJ (US); Reginald BRADLEY, Bedmister, NJ (US)

(21) Appl. No.: 17/258,545

(22) PCT Filed: Jun. 21, 2019
(86) PCT No.: PCT/US2019/038384

§ 371 Date: Jan. 7, 2021

Related U.S. Application Data
(60) Provisional application No. 62/695,589, filed on Jul. 9, 2018.

(51) Int. Cl. A61K 47/26; A61K 47/10; A61K 9/06; A61K 9/08; A61K 9/00; A61K 31/485; A61K 31/137; A61K 47/12; A61K 47/36; A61K 31/4409

(52) U.S. Cl. CPC A61K 47/26; A61K 47/10; A61K 9/06; A61K 9/08; A61K 31/4409; A61K 31/485; A61K 31/137; A61K 47/12; A61K 47/36; A61K 9/0095

(57) ABSTRACT

The present disclosure relates to an aqueous-based composition containing one or more natural polymers and two or more natural polyols, where the aqueous-based composition is formulated to inhibit microbial growth without utilizing any added artificial preservatives.

### CROSS-REFERENCE TO RELATED APPLICATIONS

This application claims priority to U.S. Provisional Application No. 62/695,589, filed Jul. 9, 2018, the disclosure of which is hereby incorporated by reference in its entirety.

### FIELD OF THE INVENTION

The present disclosure relates to an aqueous-based composition containing one or more natural polymers and two or more natural polyols, where the aqueous-based composition is formulated to inhibit microbial growth without utilizing any added artificial preservatives.

### BACKGROUND

Aqueous-based compositions are used in a wide range of pharmaceutical, nutritional, and cosmetic applications. These compositions contain multiple components that can promote microbial growth, including water, which forms the basis of these compositions, and so antimicrobial preservatives are added to ensure these compositions are safe for human use. For example, aqueous-based compositions in syrup form are orally administered to infants and toddlers as well as school-aged children, because they allow simple swallowing of pharmaceutical doses. The additives that provide the syrupy consistency and contribute to the sweetness of these compositions, however, can also generally act as substrates and provide nutrients that promote microbial growth and contamination. Most pediatric syrups, therefore, contain antimicrobial preservatives, as well as synthetic flavorants and colorants to provide a more pleasant taste and appearance. Recent studies linking preservatives and other synthetic compounds to various diseases and disorders have increased public concern about the presence of such synthetic additives in medications, foods, and cosmetics. Because aqueous-based compositions can be administered at a very young age, there is an interest in making them as free of synthetic additives as possible.

One potential approach is described by Barr and Tice (Barr, M. and Tice, L. F. (1957), Journal of the American Pharmaceutical Association (Scientific ed.) 46(4): 217-218.). However, while Barr and Tice disclose the use of a combination of glycerin and sorbitol in aqueous formulations to inhibit microbial growth, they do not disclose the further inclusion in their aqueous formulations of high levels of a natural polymer, for example, to adjust the viscosity of the aqueous formulations. It was thought to be problematic to use high levels of natural polymers in the absence of synthetic or artificial preservatives, as such high levels of natural polymers would promote microbial contamination.

### BRIEF SUMMARY

Thus, there exists a need for liquid compositions, such as syrups, that include natural polymers for adjusting viscosity, but that are free of antimicrobial preservatives, contain only natural flavorants and colorants, and are still able to maintain antimicrobial effectiveness. In order to meet these needs, the present disclosure provides aqueous-based compositions that have antimicrobial effectiveness without containing preservatives. Moreover, the present disclosure is based, at least in part, on the surprising discovery that multiple aqueous-based compositions containing known natural polyols were able to maintain antimicrobial effectiveness in the presence of high amounts of a natural polymer (See e.g., Example 1). Natural polymers, such as xanthan gum, can act as substrates and provide nutrients that promote microbial growth and contamination. Unexpectedly, known natural polyols at specific concentrations were able to overcome this quality of natural polymers and inhibit microbial growth. This finding was especially surprising in the context of an aqueous-based composition, as water is known to promote microbial growth. As such, it would have been expected that adding a microbial substrate, such as xanthan gum, to an aqueous-based composition would result in microbial contamination. Surprisingly, however, natural polyols at specific concentrations were sufficient to inhibit microbial growth. While natural polyols may have some antimicrobial efficacy in aqueous-based compositions in the absence of any microbial growth-promoting substances, it was entirely unexpected that this quality would be maintained in fully-formulated aqueous-based compositions containing such microbial growth-promoting substances. Advantageously, the examples herein demonstrate that the antimicrobial effectiveness of aqueous-based compositions having two or more natural polyols, for example glycerin and sorbitol, at a specific range of concentrations is maintained in the presence of a high level of a natural polymer, for example xanthan gum, and/or a broad pH range, without the need for any further artificial or otherwise synthetic preservatives to maintain such antimicrobial effectiveness.

Accordingly, certain aspects of the present disclosure relate to an aqueous-based composition, having one or more natural polymers; and two or more natural polyols, where the aqueous-based composition does not have any pharmaceutical antimicrobial preservatives or any artificial antimicrobial preservatives. In some embodiments, the aqueous-based composition has each of the two or more natural polyols in an amount that is effective as an antimicrobial. Other aspects of the present disclosure relate to an aqueous-based composition, having one or more natural polymers; and two or more natural polyols, where the aqueous-based composition does not have any butylparaben, methylparaben, propylparaben, EDTA, sodium benzoate, sodium propionate, or any combination thereof. In some embodiments, the aqueous-based composition has each of the two or more natural polyols in an amount that is effective as an antimicrobial. Other aspects of the present disclosure relate to an aqueous-based composition, having one or more natural polymers in an amount effective to promote microbial growth; and two or more natural polyols in an amount sufficient to inhibit the microbial growth promoted by the one or more natural polymers. In some embodiments, the aqueous-based composition includes a pharmaceutically effective amount of one or more active pharmaceutical ingredients.

Other aspects of the present disclosure relate to an aqueous-based composition, having a pharmaceutically effective amount of one or more active pharmaceutical ingredients; one or more natural polymers; and two or more natural polyols, where the aqueous-based composition does not have any pharmaceutical antimicrobial preservatives. In some embodiments, the aqueous-based composition has each of the two or more natural polyols in an amount that is effective as an antimicrobial. Other aspects of the present disclosure relate to an aqueous-based composition, having a pharmaceutically effective amount of one or more active pharmaceutical ingredients; one or more natural polymers; and two or more natural polyols, where the aqueous-based composition does not have butylparaben, methylparaben, propylparaben, EDTA, sodium benzoate, sodium propionate, or any combination thereof. In some embodiments, the aqueous-based composition has each of the two or more natural polyols in an amount that is effective as an antimicrobial. Other aspects of the present disclosure relate to an aqueous-based composition, having a pharmaceutically effective amount of one or more active pharmaceutical ingredients; one or more natural polymers; and two or more natural polyols, where the aqueous-based composition does not have any artificial antimicrobial preservatives. Other aspects of the present disclosure relate to an aqueous-based composition, having a pharmaceutically effective amount of one or more active pharmaceutical ingredients; one or more natural polymers in an amount effective to promote microbial growth; and two or more natural polyols in an amount sufficient to inhibit the microbial growth promoted by the one or more natural polymers.

In some embodiments that may be combined with any of the preceding embodiments, the one or more natural polymers are one or more polysaccharides. In some embodiments that may be combined with any of the preceding embodiments, the one or more polysaccharides are selected from xanthan gum, alginate (alginic acid), carageenan, pectin, acacia gum (gum arabic), tragacanth, locust bean gum, guar gum, cellulose gum, gellan gum, chitosan, and any combination thereof. In some embodiments that may be combined with any of the preceding embodiments, the one or more natural polymers are one or more peptides. In some embodiments that may be combined with any of the preceding embodiments, the one or more peptides are gelatin. In some embodiments that may be combined with any of the preceding embodiments, the one or more natural polymers are one or more thickening agents. In some embodiments that may be combined with any of the preceding embodiments, the one or more thickening agents are xanthan gum. In some embodiments that may be combined with any of the preceding embodiments, the aqueous-based composition contains the one or more natural polymers in an amount that ranges from 0.5% w/v to 1% w/v. In some embodiments that may be combined with any of the preceding embodiments, the two or more natural polyols are glycerin and one or more sugar alcohols. In some embodiments that may be combined with any of the preceding embodiments, the one or more sugar alcohols are selected from sorbitol, mannitol, maltitol, xylitol, erythritol, isomalt or any combination thereof. In some embodiments that may be combined with any of the preceding embodiments, the two or more natural polyols are glycerin and sorbitol. In some embodiments that may be combined with any of the preceding embodiments, the aqueous-based composition has glycerin in an amount that ranges from about 50% w/v to about 70% w/v. In some embodiments that may be combined with any of the preceding embodiments, the aqueous-based composition has sorbitol in an amount that ranges from about 10% w/v to about 50% w/v.

In some embodiments that may be combined with any of the preceding embodiments, the one or more active pharmaceutical ingredients are two or more pharmaceutical ingredients, three or more active pharmaceutical ingredients, four or more active pharmaceutical ingredients, or five or more active pharmaceutical ingredients. In some embodiments that may be combined with any of the preceding embodiments, the one or more active pharmaceutical ingredients are selected from an anti-inflammatory, an antirheumatic, an antipyretic, a decongestant, a cough-suppressant, an expectorant, an anti-allergy, an antiemetic, an analgesic, an antiepileptic, an antipsychotic, an antidepressant, a hypnotic, an anti-ulceric, a prokinetic, an anti-asthmatic, an antiparkinsonic, a cardiovascular, a vasodilator, a urologic, a diuretic, a hypolipidemic, an anti-diabetic, an anesthetic, an opioid, an opioid derivative, an antihistaminic active ingredient, and any combination thereof. In some embodiments that may be combined with any of the preceding embodiments, the one or more active pharmaceutical ingredients are selected from phenylephrine, dextromethorphan, chlorpheniramine, doxylamine, guaifenesin, loratadine, diphenhydramine, desloratadine, fexofenadine, docusate, pseudoephedrine, cetirizine, triprolidine, brompheniramine, ephedrine, ibuprofen, acetaminophen (paracetamol), ketoprofen, naproxen, piroxicam, meloxicam, leflunomide, ondansetron, granisetron, carbamazepine, lamotrigine, clozapine, olanzapine, risperidone, citalopram, paroxetine, sertraline, fluoxetine, fluvoxamine, zopiclon, zolpidem, cimetidine, ranitidine, famotidine, omeprazole, metoclopramide, cisapride, domperidon, zafirlukast, montelukast, pramipexol, selegiline, doxazosin, terazosin, atenolol, bisoprolol, amlodipine, nifedipine, diltiazem, enalapril, captopril, ramipril, losartan, glyceroltrinitrate, alfuzosin, finasteride, pravastatin, atorvastatin, simvastatin, gemfibrozil, metformin, terfenadine, celecoxib, rifecoxib, rivastignine, astemizole, hydroxyzine, clemastine, methyl salicylate, choline salicylate, aspirin, lidocaine, benzocaine, tetracaine, and a pharmaceutically acceptable salt, ester, hydrate or solvate thereof. In some embodiments that may be combined with any of the preceding embodiments, the pharmaceutically effective amount of the one or more active pharmaceutical ingredients ranges from 0.1 mg/ml to 35 mg/ml.

In some embodiments that may be combined with any of the preceding embodiments, the one or more active pharmaceutical ingredients are two pharmaceutical ingredients. In some embodiments that may be combined with any of the preceding embodiments, the two pharmaceutical ingredients are phenylephrine, or a pharmaceutically acceptable salt, ester, hydrate, or solvate thereof, and dextromethorphan, or a pharmaceutically acceptable salt, ester, hydrate, or solvate thereof. In some embodiments that may be combined with any of the preceding embodiments, the two pharmaceutical ingredients are dextromethorphan, or a pharmaceutically acceptable salt, ester, hydrate, or solvate thereof, and chlorpheniramine, or a pharmaceutically acceptable salt, ester, hydrate, or solvate thereof. In some embodiments that may be combined with any of the preceding embodiments, the two pharmaceutical ingredients are phenylephrine, or a pharmaceutically acceptable salt, ester, hydrate, or solvate thereof, and chlorpheniramine, or a pharmaceutically acceptable salt, ester, hydrate, or solvate thereof. In some embodiments that may be combined with any of the preceding embodiments, the two pharmaceutical ingredients are diphenhydramine, or a pharmaceutically acceptable salt, ester, hydrate, or solvate thereof, and phenylephrine, or a pharmaceutically acceptable salt, ester, hydrate, or solvate thereof.

Other aspects of the present disclosure relate to an aqueous-based composition, having a pharmaceutically effective amount of phenylephrine and dextromethorphan, or salts thereof; xanthan gum; glycerin; and sorbitol, where the aqueous-based composition does not have any pharmaceutical antimicrobial preservatives or any artificial antimicrobial preservatives. In some embodiments, the pharmaceutical antimicrobial preservative or artificial antimicrobial preservative is selected from butylparaben, methylparaben, propylparaben, EDTA, sodium benzoate, sodium propionate, and any combination thereof. Other aspects of the present disclosure relate to an aqueous-based composition, having a pharmaceutically effective amount of phenylephrine and dextromethorphan, or salts thereof; xanthan gum, wherein the xanthan gum is present in an amount effective to promote microbial growth; glycerin; and sorbitol, where the glycerin and sorbitol are present in amounts sufficient to inhibit the microbial growth promoted by the xanthan gum. In some embodiments, the pharmaceutically effective amount of phenylephrine ranges from 0.3 mg/ml to 3 mg/ml. In some embodiments, the pharmaceutically effective amount of dextromethorphan ranges from 1.3 mg/ml to 3 mg/ml. In some embodiments, the aqueous-based composition comprises the xanthan gum in an amount that ranges from 0.05% w/v to 1% w/v. In some embodiments, the aqueous-based composition comprises the glycerin in an amount that ranges from about 50% w/v to about 70% w/v. In some embodiments, the aqueous-based composition comprises sorbitol in an amount that ranges from about 10% w/v to about 50% w/v.

Other aspects of the present disclosure relate to an aqueous-based composition, having a pharmaceutically effective amount of dextromethorphan and chlorpheniramine, or salts thereof; xanthan gum; glycerin; and sorbitol, where the aqueous-based composition does not have any pharmaceutical antimicrobial preservatives or any artificial antimicrobial preservatives. Other aspects of the present disclosure relate to an aqueous-based composition, having a pharmaceutically effective amount of dextromethorphan and chlorpheniramine, or salts thereof xanthan gum; glycerin; and sorbitol, where the aqueous-based composition does not have butylparaben, methylparaben, propylparaben, EDTA, sodium benzoate, sodium propionate, or any combination thereof. Other aspects of the present disclosure relate to an aqueous-based composition, having a pharmaceutically effective amount of dextromethorphan and chlorpheniramine, or salts thereof xanthan gum, wherein the xanthan gum is present in an amount effective to promote microbial growth; glycerin; and sorbitol, wherein the glycerin and sorbitol are present in amounts sufficient to inhibit the microbial growth promoted by the xanthan gum. In some embodiments, the pharmaceutically effective amount of dextromethorphan ranges from 1.3 mg/ml to 3 mg/ml. In some embodiments, the pharmaceutically effective amount of chlorpheniramine ranges from 0.2 mg/ml to 2 mg/ml. In some embodiments, the aqueous-based composition comprises the xanthan gum in an amount that ranges from 0.05% w/v to 1% w/v. In some embodiments, the aqueous-based composition comprises the glycerin in an amount that ranges from about 50% w/v to about 70% w/v. In some embodiments, the aqueous-based composition comprises sorbitol in an amount that ranges from about 10% w/v to about 50% w/v.

Other aspects of the present disclosure relate to an aqueous-based composition, having a pharmaceutically effective amount of phenylephrine and chlorpheniramine, or salts thereof xanthan gum; glycerin; and sorbitol, where the aqueous-based composition does not have any pharmaceutical antimicrobial preservatives or any artificial antimicrobial preservatives. Other aspects of the present disclosure relate to an aqueous-based composition, having a pharmaceutically effective amount of phenylephrine and chlorpheniramine, or salts thereof xanthan gum; glycerin; and sorbitol, where the aqueous-based composition does not have butylparaben, methylparaben, propylparaben, EDTA, sodium benzoate, sodium propionate, or any combination thereof. Other aspects of the present disclosure relate to an aqueous-based composition, having a pharmaceutically effective amount of phenylephrine and chlorpheniramine, or salts thereof; xanthan gum, wherein the xanthan gum is present in an amount effective to promote microbial growth; glycerin; and sorbitol, where the glycerin and sorbitol are present in amounts sufficient to inhibit the microbial growth promoted by the xanthan gum. In some embodiments, the pharmaceutically effective amount of phenylephrine ranges from 0.3 mg/ml to 3 mg/ml. In some embodiments, the pharmaceutically effective amount of chlorpheniramine ranges from 0.2 mg/ml to 2 mg/ml. In some embodiments, the aqueous-based composition comprises the xanthan gum in an amount that ranges from 0.05% w/v to 1% w/v. In some embodiments, the aqueous-based composition comprises the glycerin in an amount that ranges from about 50% w/v to about 70% w/v. In some embodiments, the aqueous-based composition comprises sorbitol in an amount that ranges from about 10% w/v to about 50% w/v.

Other aspects of the present disclosure relate to an aqueous-based composition, having a pharmaceutically effective amount of diphenhydramine and chlorpheniramine, or salts thereof; xanthan gum; glycerin; and sorbitol, where the aqueous-based composition does not have any pharmaceutical antimicrobial preservatives or any artificial antimicrobial preservatives. Other aspects of the present disclosure relate to an aqueous-based composition, having a pharmaceutically effective amount of diphenhydramine and chlorpheniramine, or salts thereof; xanthan gum; glycerin; and sorbitol, where the aqueous-based composition does not have butylparaben, methylparaben, propylparaben, EDTA, sodium benzoate, sodium propionate, or any combination thereof. Other aspects of the present disclosure relate to an aqueous-based composition, having a pharmaceutically effective amount of diphenhydramine and chlorpheniramine, or salts thereof; xanthan gum, where the xanthan gum is present in an amount effective to promote microbial growth; glycerin; and sorbitol, where the glycerin and sorbitol are present in amounts sufficient to inhibit the microbial growth promoted by the xanthan gum. In some embodiments, the pharmaceutically effective amount of diphenhydramine ranges from 1.5 mg/ml to 7.5 mg/ml. In some embodiments, the pharmaceutically effective amount of chlorpheniramine ranges from 0.2 mg/ml to 2 mg/ml. In some embodiments, the aqueous-based composition comprises the xanthan gum in an amount that ranges from 0.05% w/v to 1% w/v. In some embodiments, the aqueous-based composition comprises the glycerin in an amount that ranges from about 50% w/v to about 70% w/v. In some embodiments, the aqueous-based composition comprises sorbitol in an amount that ranges from about 10% w/v to about 50% w/v.

In some embodiments that may be combined with any of the preceding embodiments, the aqueous-based composition has one or more pharmaceutically acceptable excipients. In some embodiments that may be combined with any of the preceding embodiments, the aqueous-based composition has a pH value that ranges from about 2.5 to about 8.0. In some embodiments that may be combined with any of the preceding embodiments, the aqueous-based composition does not have an artificial flavorant. In some embodiments that may be combined with any of the preceding embodiments, the aqueous-based composition has a natural flavorant selected from a honey flavorant, a grape flavorant, a strawberry flavorant, a raspberry flavorant, a blue raspberry flavorant, and a tropical punch flavorant. In some embodiments that may be combined with any of the preceding embodiments, the aqueous-based composition does not have an artificial colorant. In some embodiments that may be combined with any of the preceding embodiments, the aqueous-based composition has a natural colorant selected from lutein, paprika, cochineal/carmine, beetroot red, anthocyanin, caramel/malt, and any combination thereof. In some embodiments that may be combined with any of the preceding embodiments, the aqueous-based composition does not have an artificial sweetener. In some embodiments that may be combined with any of the preceding embodiments, the aqueous-based composition is in the form of a solution, a syrup, a suspension, or a gel. In some embodiments that may be combined with any of the preceding embodiments, the aqueous-based composition is a multi-dose form. In some embodiments that may be combined with any of the preceding embodiments, the aqueous-based composition is an oral composition, a buccal composition, a sublingual composition, an inhalation composition, a nebulization composition, an ophthalmic composition, an otic composition, a nasal composition, an intravenous composition, an intramuscular composition, an intrathecal composition, an intraosseous composition, a rectal composition, a subcutaneous composition, a cutaneous composition, or a transdermal composition. In some embodiments that may be combined with any of the preceding embodiments, the aqueous-based composition is an oral composition.

Other aspects of the present disclosure relate to a liquid multi-dosage form, having one or more natural polymers in an amount effective to promote microbial growth, where the liquid multi-dosage form does not have any pharmaceutical antimicrobial preservatives or any artificial antimicrobial preservatives. Other aspects of the present disclosure relate to a liquid multi-dosage form, having one or more natural polymers in an amount effective to promote microbial growth, where the liquid multi-dosage form does not have butylparaben, methylparaben, propylparaben, EDTA, sodium benzoate, sodium propionate, or any combination thereof. In some embodiments, the liquid multi-dosage form has two or more natural polyols in an amount sufficient to inhibit the microbial growth promoted by the one or more natural polymers. In some embodiments, the two or more natural polyols are glycerin and one or more sugar alcohols. In some embodiments, the one or more sugar alcohols are selected from sorbitol, mannitol, maltitol, xylitol, erythritol, isomalt or any combination thereof. In some embodiments, the two or more natural polyols are glycerin and sorbitol. In some embodiments, the liquid multi-dosage form contains glycerin in an amount that ranges from about 50% w/v to about 70% w/v. In some embodiments, the liquid multi-dosage form contains sorbitol in an amount that ranges from about 10% w/v to about 50% w/v. In some embodiments, the liquid multi-dosage form further contains a pharmaceutically effective amount of one or more active pharmaceutical ingredients. In some embodiments, the one or more active pharmaceutical ingredients are two or more pharmaceutical ingredients, three or more active pharmaceutical ingredients, four or more active pharmaceutical ingredients, or five or more active pharmaceutical ingredients. In some embodiments, the one or more active pharmaceutical ingredients are selected from an anti-inflammatory, an antirheumatic, an antipyretic, a decongestant, a cough-suppressant, an expectorant, an anti-allergy, an antiemetic, an analgesic, an antiepileptic, an antipsychotic, an antidepressant, a hypnotic, an anti-ulceric, a prokinetic, an anti-asthmatic, an antiparkinsonic, a cardiovascular, a vasodilator, a urologic, a diuretic, a hypolipidemic, an anti-diabetic, an anesthetic, an opioid, an opioid derivative, an antihistaminic active ingredient, and any combination thereof. In some embodiments, the one or more active pharmaceutical ingredients are selected from phenylephrine, dextromethorphan, chlorpheniramine, doxylamine, guaifenesin, loratadine, diphenhydramine, desloratadine, fexofenadine, docusate, pseudoephedrine, cetirizine, triprolidine, brompheniramine, ephedrine, ibuprofen, acetaminophen (paracetamol), ketoprofen, naproxen, piroxicam, meloxicam, leflunomide, ondansetron, granisetron, carbamazepine, lamotrigine, clozapine, olanzapine, risperidone, citalopram, paroxetine, sertraline, fluoxetine, fluvoxamine, zopiclon, zolpidem, cimetidine, ranitidine, famotidine, omeprazole, metoclopramide, cisapride, domperidon, zafirlukast, montelukast, pramipexol, selegiline, doxazosin, terazosin, atenolol, bisoprolol, amlodipine, nifedipine, diltiazem, enalapril, captopril, ramipril, losartan, glyceroltrinitrate, alfuzosin, finasteride, pravastatin, atorvastatin, simvastatin, gemfibrozil, metformin, terfenadine, celecoxib, rifecoxib, rivastignine, astemizole, hydroxyzine, clemastine, methyl salicylate, choline salicylate, aspirin, lidocaine, benzocaine, tetracaine, and a pharmaceutically acceptable salt, ester, hydrate or solvate thereof. In some embodiments, the pharmaceutically effective amount of the one or more active pharmaceutical ingredients ranges from 0.1 mg/ml to 35 mg/ml. In some embodiments, the one or more active pharmaceutical ingredients are two pharmaceutical ingredients. In some embodiments, the two pharmaceutical ingredients are phenylephrine, or a pharmaceutically acceptable salt, ester, hydrate, or solvate thereof, and dextromethorphan, or a pharmaceutically acceptable salt, ester, hydrate, or solvate thereof. In some embodiments, the two pharmaceutical ingredients are dextromethorphan, or a pharmaceutically acceptable salt, ester, hydrate, or solvate thereof, and chlorpheniramine, or a pharmaceutically acceptable salt, ester, hydrate, or solvate thereof. In some embodiments, the two pharmaceutical ingredients are phenylephrine, or a pharmaceutically acceptable salt, ester, hydrate, or solvate thereof, and chlorpheniramine, or a pharmaceutically acceptable salt, ester, hydrate, or solvate thereof. In some embodiments, the two pharmaceutical ingredients are diphenhydramine, or a pharmaceutically acceptable salt, ester, hydrate, or solvate thereof, and phenylephrine, or a pharmaceutically acceptable salt, ester, hydrate, or solvate thereof. In some embodiments that may be combined with any of the preceding embodiments, the one or more natural polymers are selected from xanthan gum, alginate (alginic acid), sodium alginate, carageenan, gelatin, pectin, acacia gum (gum arabic), tragacanth, locust bean gum, guar gum, cellulose gum, gellan gum, chitosan or any combination thereof. In some embodiments that may be combined with any of the preceding embodiments, the one or more natural polymers are one or more thickening agents. In some embodiments that may be combined with any of the preceding embodiments, the one or more thickening agents are xanthan gum. In some embodiments that may be combined with any of the preceding embodiments, the liquid multi-dosage form has the one or more natural polymers in an amount that ranges from 0.5% w/v to 1% w/v. In some embodiments that may be combined with any of the preceding embodiments, the liquid multi-dosage form contains one or more pharmaceutically acceptable excipients. In some embodiments that may be combined with any of the preceding embodiments, the liquid multi-dosage form has a pH value that ranges from about 2.5 to about 8.0. In some embodiments that may be combined with any of the preceding embodiments, the liquid multi-dosage form does not have any artificial flavorant. In some embodiments that may be combined with any of the preceding embodiments, the liquid multi-dosage form contains a natural flavorant selected from a honey flavorant, a grape flavorant, a strawberry flavorant, a raspberry flavorant, a blue raspberry flavorant, and a tropical punch flavorant. In some embodiments that may be combined with any of the preceding embodiments, the liquid multi-dosage form does not contain any artificial colorant. In some embodiments that may be combined with any of the preceding embodiments, the liquid multi-dosage form contains a natural colorant selected from lutein, paprika, cochineal/carmine, beetroot red, anthocyanin, caramel/malt, and any combination thereof. In some embodiments that may be combined with any of the preceding embodiments, the liquid multi-dosage form does not contain an artificial sweetener. In some embodiments that may be combined with any of the preceding embodiments, the liquid multi-dosage form is in the form of a solution, a syrup, or a suspension. In some embodiments that may be combined with any of the preceding embodiments, the liquid multi-dosage form is an oral composition, a buccal composition, a sublingual composition, an inhalation composition, a nebulization composition, an ophthalmic composition, an otic composition, a nasal composition, an intravenous composition, an intramuscular composition, an intrathecal composition, an intraosseous composition, a rectal composition, a subcutaneous composition, a cutaneous composition, or a transdermal composition.

It is to be understood that one, some, or all of the properties of the various embodiments described herein may be combined to form other embodiments of the present invention. These and other aspects of the invention will become apparent to one of skill in the art.

### DETAILED DESCRIPTION

The following description sets forth exemplary methods, parameters, and the like. It should be recognized, however, that such description is not intended as a limitation on the scope of the present disclosure but is instead provided as a description of exemplary embodiments.

### Compositions

Certain aspects of the present disclosure relate to aqueous-based compositions containing one or more natural polymers and two or more natural polyols. As used herein, the term “aqueous-based” refers to a composition that is primarily water-based, but may include any number of active ingredients and/or excipients, and can be in the form of, e.g., a solution, a syrup, a suspension, a gel, etc. In some embodiments, aqueous-based compositions of the present disclosure do not contain a pharmaceutical antimicrobial or an artificial antimicrobial preservative. In some embodiments, the aqueous-based oral compositions may contain one or more natural polymers that promote microbial growth and two or more natural polyols in an amount sufficient to inhibit the microbial growth promoted by the one or more natural polymers. As used herein, “microbes” refer to bacteria, molds, and yeasts. Examples of microbes include, but are not limited to, the test organisms that may be used in antimicrobial effectiveness testing, such as Candida albicans, Aspergillus brasiliensis, Escherichia coli, Pseudomonas aeruginosa, and Staphylococcus aureus. In some embodiments, the aqueous-based compositions further contain a pharmaceutically effective amount of one or more active pharmaceutical ingredients (APIs). In some embodiments, the aqueous-based compositions may contain a pharmaceutically effective amount of phenylephrine and dextromethorphan, the natural polymer xanthan gum, the natural polyols glycerin and sorbitol, and do not contain a pharmaceutical antimicrobial or an artificial antimicrobial preservative. In some embodiments, the aqueous-based compositions may contain a pharmaceutically effective amount of phenylephrine and dextromethorphan, the natural polymer xanthan gum that promotes microbial growth, and the natural polyols glycerin and sorbitol in an amount sufficient to inhibit the microbial growth promoted by the natural polymer xanthan gum. The aqueous-based compositions may include, without limitation, one or more natural polymers, two or more natural polyols, one or more excipients, one or more thickening agents, one or more pH-adjusting agents, one or more natural sweeteners, one or more natural flavorants, one or more natural colorants, and/or water.

An aqueous-based composition of the present disclosure can be any suitable composition. A suitable composition may include, without limitation, a pharmaceutical composition, a nutritional composition, and/or a cosmetic composition. In some embodiments, the pharmaceutical composition may be, without limitation, a medicated liquid or syrup, a medicated gel or paste, and/or a medicated lotion or cream. In some embodiments, the pharmaceutical composition may be delivered via a spray, for example using an aspirator. In some embodiments, the pharmaceutical composition may be delivered via drops, for example using a squeezable bottle. In some embodiments, the pharmaceutical composition may be orally administered to children in the form of a liquid or syrup. In some embodiments, the nutritional composition may be, without limitation, a food, a beverage or a substance used in the preparation thereof, an energy gel or paste, and/or a nutritional supplement such as a multivitamin. In some embodiments, the nutritional composition may be formulated to fit the dietary requirements of children. In some embodiments, the cosmetic composition may be, without limitation, any suitable lotion, gel, or cream known in the art. In some embodiments, the cosmetic composition may be any suitable hair treatment, for example shampoo or conditioner. In some embodiments, the cosmetic composition may be any suitable deodorant, shaving cream, and/or soap. In some embodiments, the cosmetic composition may be makeup, for example foundation or lipstick.

A suitable composition of an aqueous-based composition of the present disclosure can be in any suitable form. A suitable form may include, without limitation, a liquid form, a syrup form, a spray form, a gel form, a paste form, a lotion form, and/or a cream form. In some embodiments, the suitable form may have a firm and gel-like consistency, may have a syrupy consistency, and/or may have a watery consistency. In some embodiments, the suitable form may have a pleasant mouthfeel, a thickened syrupy consistency, and/or may be easily swallowed.

Natural Polymers

In some aspects, the present disclosure relates to aqueous-based compositions containing one or more (e.g., one or more, two or more, three or more, four or more, five or more, etc.) natural polymers. In some embodiments, the one or more natural polymers are known to promote microbial growth. In some embodiments, the one or more natural polymers increase the viscosity of the aqueous-based composition. In some embodiments, the one or more natural polymers are thickening agents. The one or more natural polymers may be any suitable natural polymer. The one or more natural polymers may be polysaccharides, proteins, peptides, glycoproteins, or a mixture thereof. Examples of suitable natural polymers include, without limitation, xanthan gum, alginate (alginic acid, sodium alginate, etc.), carageenan, gelatin, pectin, acacia gum (gum arabic), tragacanth, locust bean gum, guar gum, cellulose gum, gellan gum, chitosan or any combination thereof. In some embodiments, the natural polymer is xanthan gum.

In some embodiments, the one or more natural polymers in the aqueous-based composition are present in an amount sufficient to produce a composition with sufficient viscosity. In some embodiments, this sufficient viscosity is suitable for syrup formulation, provides a pleasant mouthfeel, and/or enables easier swallowing. Accordingly, in some embodiments, the aqueous-based composition contains one or more natural polymers in an amount sufficient to provide the composition with the desired viscosity for a syrup formulation and/or a pleasant mouthfeel, sufficient to allow easier swallowing of the composition.

In some embodiments, the aqueous-based composition contains one or more natural polymers in an amount that ranges from about 0.5% w/v to about 1% w/v. In some embodiments, the aqueous-based composition contains the one or more natural polymers in an amount of at least about 0.5% w/v, at least about 0.55% w/v, at least about 0.6% w/v, at least about 0.65% w/v, at least about 0.7% w/v, at least about 0.75% w/v, at least about 0.8% w/v, at least about 0.85% w/v, at least about 0.9% w/v, at least about 0.91% w/v, at least about 0.92% w/v, at least about 0.93% w/v, at least about 0.94% w/v, at least about 0.95% w/v, at least about 0.96% w/v, at least about 0.97% w/v, at least about 0.98% w/v, at least about 0.99% w/v, or at least about 1% w/v.

As used herein, the term “percent weight/volume (% w/v)” refers to the weight of the indicated component as a percentage of the total volume of the final formulation of an individual aqueous-based composition of the present disclosure.

In some embodiments, the aqueous-based composition includes one or more natural polymers in an amount sufficient to yield a composition having a dynamic viscosity in the range of 30 to 5000 centipoise (cP) at room temperature when measured using a LV2 spindle using a viscometer at 30 rpm. In some embodiments, the dynamic viscosity is at least about 30 cP, at least about 50 cP, at least about 100 cP, at least about 150 cP, at least about 200 cP, at least about 250 cP, at least about 300 cP, at least about 350 cP, at least about 400 cP, at least about 450 cP, at least about 500 cP, at least about 550 cP, at least about 600 cP, at least about 650 cP, at least about 700 cP, at least about 750 cP, at least about 800 cP, at least about 850 cP, at least about 900 cP, at least about 950 cP, at least about 1000 cP, at least about 1050 cP, at least about 1100 cP, at least about 1150 cP, at least about 1200 cP, at least about 1250 cP, at least about 1300 cP, at least about 1350 cP, at least about 1400 cP, at least about 1450 cP, at least about 1500 cP, at least about 1550 cP, at least about 1600 cP, at least about 1650 cP, at least about 1700 cP, at least about 1750 cP, at least about 1800 cP, at least about 1850 cP, at least about 1900 cP, at least about 1950 cP, at least about 2000 cP, at least about 2500 cP, at least about 3000 cP, at least about 3500 cP, at least about 4000 cP, at least about 4500 cP, or at least about 5000 cP.

Natural Polyols

In some aspects, the present disclosure relates to aqueous-based compositions containing two or more natural polyols (e.g., two or more, three or more, four or more, five or more, six or more, etc.). As disclosed herein, the two or more natural polyols are present in an amount sufficient to inhibit microbial growth. Any suitable natural polyol may be used. In some embodiments, one of the two or more natural polyols may be a sugar alcohol. Examples of suitable natural polyols include, without limitation, glycerin, sorbitol, mannitol, maltitol solution, xylitol, erythritol, isomalt and any combination thereof. In some embodiments, the two or more natural polyols are glycerin and sorbitol. In some embodiments, the two or more natural polyols are glycerin and maltitol. In some embodiments, the two or more natural polyols are glycerin plus one or more sugar alcohols.

In some embodiments, the aqueous-based composition contains two or more natural polyols in an amounts sufficient to inhibit microbial growth. In some embodiments, the aqueous-based composition contains two or more natural polyols, wherein each natural polyol is present in an amount that ranges from about 10% w/v to about 70% w/v. In some embodiments, the aqueous-based composition contains two or more natural polyols, wherein each natural polyol is present in an amount that is less than about 70% w/v, less than about 69% w/v, less than about 68% w/v, less than about 67% w/v, less than about 66% w/v, less than about 65% w/v, less than about 64% w/v, less than about 63% w/v, less than about 62% w/v, less than about 61% w/v, less than about 60% w/v, less than about 59% w/v, less than about 58% w/v, less than about 57% w/v, less than about 56% w/v, less than about 55% w/v, less than about 54% w/v, less than about 53% w/v, less than about 52% w/v, less than about 51% w/v, less than about 50% w/v, less than about 49% w/v, less than about 48% w/v, less than about 47% w/v, less than about 46% w/v, less than about 45% w/v, less than about 44% w/v, less than about 43% w/v, less than about 42% w/v, less than about 41% w/v, less than about 40% w/v, less than about 39% w/v, less than about 38% w/v, less than about 37% w/v, less than about 36% w/v, less than about 35% w/v, less than about 34% w/v, less than about 33% w/v, less than about 32% w/v, less than about 31% w/v, less than about 30% w/v, less than about 29% w/v, less than about 28% w/v, less than about 27% w/v, less than about 26% w/v, less than about 25% w/v, less than about 24% w/v, less than about 23% w/v, less than about 22% w/v, less than about 21% w/v, less than about 20% w/v, less than about 19% w/v, less than about 18% w/v, less than about 17% w/v, less than about 16% w/v, less than about 15% w/v, less than about 14% w/v, less than about 13% w/v, less than about 12% w/v, less than about 11% w/v, or less than about 10% w/v. In some embodiments, the aqueous-based composition contains two or more natural polyols, wherein each natural polyol is present in an amount that is greater than about 10% w/v, greater than about 11% w/v, greater than about 12% w/v, greater than about 13% w/v, greater than about 14% w/v, greater than about 15% w/v, greater than about 16% w/v, greater than about 17% w/v, greater than about 18% w/v, greater than about 19% w/v, greater than about 20% w/v, greater than about 21% w/v, greater than about 22% w/v, greater than about 23% w/v, greater than about 24% w/v, greater than about 25% w/v, greater than about 26% w/v, greater than about 27% w/v, greater than about 28% w/v, greater than about 29% w/v, greater than about 30% w/v, greater than about 31% w/v, greater than about 32% w/v, greater than about 33% w/v, greater than about 34% w/v, greater than about 35% w/v, greater than about 36% w/v, greater than about 37% w/v, greater than about 38% w/v, greater than about 39% w/v, greater than about 40% w/v, greater than about 41% w/v, greater than about 42% w/v, greater than about 43% w/v, greater than about 44% w/v, greater than about 45% w/v, greater than about 46% w/v, greater than about 47% w/v, greater than about 48% w/v, greater than about 49% w/v, greater than about 50% w/v, greater than about 51% w/v, greater than about 52% w/v, greater than about 53% w/v, greater than about 54% w/v, greater than about 55% w/v, greater than about 56% w/v, greater than about 57% w/v, greater than about 58% w/v, greater than about 59% w/v, greater than about 60% w/v, greater than about 61% w/v, greater than about 62% w/v, greater than about 63% w/v, greater than about 64% w/v, greater than about 65% w/v, greater than about 66% w/v, greater than about 67% w/v, greater than about 68% w/v, greater than about 69% w/v, or greater than about 70% w/v.

In some embodiments, the aqueous-based composition contains glycerin in an amount that ranges from about 50% w/v to about 70% w/v. In some embodiments, the aqueous-based composition contains glycerin in an amount that is less than about 70% w/v, less than about 69.5% w/v, less than about 69% w/v, less than about 68.5% w/v, less than about 68% w/v, less than about 67.5% w/v, less than about 67% w/v, less than about 66.5% w/v, less than about 66% w/v, less than about 65.5% w/v, less than about 65% w/v, less than about 64.5% w/v, less than about 64% w/v, less than about 63.5% w/v, less than about 63% w/v, less than about 62.5% w/v, less than about 62% w/v, less than about 61.5% w/v, less than about 61% w/v, less than about 60.5% w/v, less than about 60% w/v, less than about 59.5% w/v, less than about 59% w/v, less than about 58.5% w/v, less than about 58% w/v, less than about 57.5% w/v, less than about 57% w/v, less than about 56.5% w/v, less than about 56% w/v, less than about 55.5% w/v, less than about 55% w/v, less than about 54.5% w/v, less than about 54% w/v, less than about 53.5% w/v, less than about 53% w/v, less than about 52.5% w/v, less than about 52% w/v, less than about 51.5% w/v, less than about 51% w/v, less than about 50.5% w/v, or less than about 50% w/v. In some embodiments, the aqueous-based composition contains glycerin in an amount that is greater than about 50% w/v, greater than about 50.5% w/v, greater than about 51% w/v, greater than about 51.5% w/v, greater than about 52% w/v, greater than about 52.5% w/v, greater than about 53% w/v, greater than about 53.5% w/v, greater than about 54% w/v, greater than about 54.5% w/v, greater than about 55% w/v, greater than about 55.5% w/v, greater than about 56% w/v, greater than about 56.5% w/v, greater than about 57% w/v, greater than about 57.5% w/v, greater than about 58% w/v, greater than about 58.5% w/v, greater than about 59% w/v, greater than about 59.5% w/v, greater than about 60% w/v, greater than about 60.5% w/v, greater than about 61% w/v, greater than about 61.5% w/v, greater than about 62% w/v, greater than about 62.5% w/v, greater than about 63% w/v, greater than about 63.5% w/v, greater than about 64% w/v, greater than about 64.5% w/v, greater than about 65% w/v, greater than about 65.5% w/v, greater than about 66% w/v, greater than about 66.5% w/v, greater than about 67% w/v, greater than about 67.5% w/v, greater than about 68% w/v, greater than about 68.5% w/v, greater than about 69% w/v, greater than about 69.5% w/v, or greater than about 70% w/v. In some embodiments, the aqueous-based composition contains glycerin in an amount that has a range of % w/v having an upper limit of about 70% w/v, 67.5% w/v, 65% w/v, 62.5% w/v, 60% w/v, 57.5% w/v, 55% w/v, 52.5% w/v, 50% w/v, and an independently selected lower limit of about 50% w/v, 52.5% w/v, 55% w/v, 57.5% w/v, 60% w/v, 62.5% w/v, 65% w/v, 67.5% w/v, or 70% w/v, where the lower limit is less than the upper limit. In some embodiments, the aqueous-based composition contains glycerin in an amount of about 50% w/v, about 52.5% w/v, about 55% w/v, about 57.5% w/v, about 60% w/v, about 62.5% w/v, about 65% w/v, about 67.5% w/v, or about 70%.

In some embodiments, the aqueous-based composition contains sorbitol in an amount that ranges from about 10% w/v to about 50% w/v. In some embodiments, the aqueous-based composition contains sorbitol in an amount that is less than about 50% w/v, less than about 49% w/v, less than about 48% w/v, less than about 47% w/v, less than about 46% w/v, less than about 45% w/v, less than about 44% w/v, less than about 43% w/v, less than about 42% w/v, less than about 41% w/v, less than about 40% w/v, less than about 39% w/v, less than about 38% w/v, less than about 37% w/v, less than about 36% w/v, less than about 35% w/v, less than about 34% w/v, less than about 33% w/v, less than about 32% w/v, less than about 31% w/v, less than about 30% w/v, less than about 29% w/v, less than about 28% w/v, less than about 27% w/v, less than about 26% w/v, less than about 25% w/v, less than about 24% w/v, less than about 23% w/v, less than about 22% w/v, less than about 21% w/v, less than about 20% w/v, less than about 19% w/v, less than about 18% w/v, less than about 17% w/v, less than about 16% w/v, less than about 15% w/v, less than about 14% w/v, less than about 13% w/v, less than about 12% w/v, less than about 11% w/v, or less than about 10% w/v. In some embodiments, the aqueous-based composition contains sorbitol in an amount that is greater than about 10% w/v, greater than about 11% w/v, greater than about 12% w/v, greater than about 13% w/v, greater than about 14% w/v, greater than about 15% w/v, greater than about 16% w/v, greater than about 17% w/v, greater than about 18% w/v, greater than about 19% w/v, greater than about 20% w/v, greater than about 21% w/v, greater than about 22% w/v, greater than about 23% w/v, greater than about 24% w/v, greater than about 25% w/v, greater than about 26% w/v, greater than about 27% w/v, greater than about 28% w/v, greater than about 29% w/v, greater than about 30% w/v, greater than about 31% w/v, greater than about 32% w/v, greater than about 33% w/v, greater than about 34% w/v, greater than about 35% w/v, greater than about 36% w/v, greater than about 37% w/v, greater than about 38% w/v, greater than about 39% w/v, greater than about 40% w/v, greater than about 41% w/v, greater than about 42% w/v, greater than about 43% w/v, greater than about 44% w/v, greater than about 45% w/v, greater than about 46% w/v, greater than about 47% w/v, greater than about 48% w/v, greater than about 49% w/v, or greater than about 50% w/v. In some embodiments, the aqueous-based composition contains sorbitol in an amount that has a range of % w/v having an upper limit of about 50% w/v, 47.5% w/v, 45% w/v, 42.5% w/v, 40% w/v, 37.5% w/v, 35% w/v, 32.5% w/v, 30% w/v, 27.5% w/v, 25% w/v, 22.5% w/v, 20% w/v, 17.5% w/v, 15% w/v, 12.5% w/v, or 10% w/v, and an independently selected lower limit of about 10% w/v, 12.5% w/v, 15% w/v, 17.5% w/v, 20% w/v, 22.5% w/v, 25% w/v, 27.5% w/v, 30% w/v, 32.5% w/v, 35% w/v, 37.5% w/v, 40% w/v, 42.5% w/v, 45% w/v, 47.5% w/v, or 50% w/v, where the lower limit is less than the upper limit. In some embodiments, the aqueous-based composition contains sorbitol in an amount of about 10% w/v, about 12.5% w/v, about 15% w/v, about 17.5% w/v, about 20% w/v, about 22.5% w/v, about 25% w/v, about 27.5% w/v, about 30%, about 32.5% w/v, about 35% w/v, about 37.5% w/v, about 40% w/v, about 42.5% w/v, about 45% w/v, about 47.5% w/v, or about 50%.

Antimicrobial Preservatives

In some aspects, the present disclosure relates to aqueous-based compositions that do not contain any pharmaceutical, artificial, or otherwise synthetic antimicrobial preservatives. As used herein, the terms “pharmaceutical antimicrobial preservative”, “artificial antimicrobial preservative”, and “synthetic antimicrobial preservative” may be used interchangeably and refer to a substance or product formed through specialized processes and requiring human agency that is added to a nonsterile aqueous-based composition to protect it from microbial growth. Pharmaceutical, artificial, or otherwise synthetic antimicrobial preservatives of the present disclosure exclude synthetic sugars, synthetic salts, synthetic glycols, synthetic alcohols, tocopherol, hops, diatomaceous earth, and castor oil.

Examples of such pharmaceutical, artificial, or otherwise synthetic antimicrobial preservatives, include, without limitation, butylparaben, methylparaben, propylparaben, EDTA, sodium benzoate, and sodium propionate.

Active Pharmaceutical Ingredients

In some aspects, the present disclosure relates to aqueous-based compositions containing one or more (e.g., one or more, two or more, three or more, four or more, five or more, etc.) active pharmaceutical ingredients (APIs).

Any suitable API may be used. Examples of suitable APIs include, without limitation, one or more of an anti-inflammatory, an antirheumatic, an antipyretic, a decongestant, a cough-suppressant, an expectorant, an anti-allergy, an antiemetic, an analgesic, an antiepileptic, an antipsychotic, an antidepressant, a hypnotic, an anti-ulceric, a prokinetic, an anti-asthmatic, an antiparkinsonic, a cardiovascular, a vasodilator, a urologic, a diuretic, a hypolipidemic, an anti-diabetic, an anesthetic, an opioid, an opioid derivative, an antihistamine active ingredient, and any combinations thereof.

In some embodiments, the one or more active pharmaceutical ingredients (APIs) of the present disclosure may include, without limitation, phenylephrine, dextromethorphan, chlorpheniramine, doxylamine, guaifenesin, loratadine, diphenhydramine, desloratadine, fexofenadine, docusate, pseudoephedrine, cetirizine, triprolidine, brompheniramine, ephedrine, ibuprofen, acetaminophen (paracetamol), ketoprofen, naproxen, piroxicam, meloxicam, leflunomide, ondansetron, granisetron, carbamazepine, lamotrigine, clozapine, olanzapine, risperidone, citalopram, paroxetine, sertraline, fluoxetine, fluvoxamine, zopiclon, zolpidem, cimetidine, ranitidine, famotidine, omeprazole, metoclopramide, cisapride, domperidon, zafirlukast, montelukast, pramipexol, selegiline, doxazosin, terazosin, atenolol, bisoprolol, amlodipine, nifedipine, diltiazem, enalapril, captopril, ramipril, losartan, glyceroltrinitrate, alfuzosin, finasteride, pravastatin, atorvastatin, simvastatin, gemfibrozil, metformin, terfenadine, celecoxib, rifecoxib, rivastignine, astemizole, hydroxyzine, clemastine, methyl salicylate, choline salicylate, aspirin, lidocaine, benzocaine, tetracaine, and a pharmaceutically acceptable salt, ester, hydrate or solvate thereof.

The quantity of the active pharmaceutical ingredients present in the suspension may be expressed in terms of concentration of milligrams per milliliter (mg/ml) of the suspension. Alternatively, the quantity of the active pharmaceutical ingredients may also be expressed as a weight percentage of the total volume of the aqueous-based composition (% w/v). As disclosed herein, the concentration of any of the active pharmaceutical ingredients of the present disclosure may be in mg/ml or % w/v.

In some embodiments, the aqueous-based composition contains one or more APIs in a therapeutically effective amount. In some embodiments, the aqueous-based composition contains one or more APIs in an amount that ranges from about 0.01% w/v to about 50% w/v. In some embodiments, the aqueous-based composition contains one or more APIs in an amount that is less than about 50% w/v, less than about 40% w/v, less than about 35% w/v, less than about 30% w/v, less than about 25% w/v, less than about 20% w/v, less than about 15% w/v, less than about 10% w/v, less than about 9% w/v, less than about 8% w/v, less than about 7% w/v, less than about 6% w/v, less than about 5% w/v, less than about 4% w/v, less than about 3% w/v, less than about 2% w/v, less than about 1% w/v, less than about 0.5% w/v, less than about 0.1% w/v, less than about 0.05% w/v, or less than about 0.01% w/v.

In some embodiments, the aqueous-based composition contains one or more APIs in an amount that is greater than about 0.01% w/v, greater than about 0.05% w/v, greater than about 0.1% w/v, greater than about 0.5% w/v, greater than about 1% w/v, greater than about 2% w/v, greater than about 3% w/v, greater than about 4% w/v, greater than about 5% w/v, greater than about 6% w/v, greater than about 7% w/v, greater than about 8% w/v, greater than about 9% w/v, greater than about 10% w/v, greater than about 15% w/v, greater than about 20% w/v, greater than about 25% w/v, greater than about 30% w/v, greater than about 35% w/v, greater than about 40% w/v, or greater than about 45% w/v.

In some embodiments, the aqueous-based composition contains one or more APIs in an amount that has a range of % w/v having an upper limit of about 50% w/v, 45% w/v, 40% w/v, 35% w/v, 30% w/v, 25% w/v, 20% w/v, 15% w/v, 10% w/v, 9% w/v, 8% w/v, 7% w/v, 6% w/v, 5% w/v, 4% w/v, 3% w/v, 2% w/v, 1% w/v, 0.5% w/v, or 0.1% w/v, and an independently selected lower limit of about 0.01% w/v, 0.05% w/v, 0.1% w/v, 0.5% w/v, 1% w/v, 2% w/v, 3% w/v, 4% w/v, 5% w/v, 6% w/v, 7% w/v, 8% w/v, 9% w/v, 10% w/v, 15% w/v, 20% w/v, 25% w/v, 30% w/v, 35% w/v, 40% w/v, or 45% w/v, where the lower limit is less than the upper limit.

In some embodiments, the aqueous-based composition contains one or more APIs in an amount of about 0.01% w/v, about 0.05% w/v, about 0.1% w/v, about 0.25% w/v, about 0.5% w/v, about 0.75% w/v, about 1% w/v, about 2% w/v, about 3% w/v, about 4% w/v, about 5% w/v, about 6% w/v, about 7% w/v, about 8% w/v, about 9% w/v, about 10% w/v, about 15% w/v, about 20% w/v, about 25% w/v, about 30% w/v, about 35% w/v, about 40% w/v, about 45% w/v, or about 50% w/v.

In some embodiments, the aqueous-based composition contains one or more APIs in a therapeutically effective amount. In some embodiments, the aqueous-based composition contains one or more APIs in an amount that ranges from about 0.1% w/v to about 5% w/v. In some embodiments, the aqueous-based composition contains one or more APIs in an amount that is less than about 5% w/v, less than about 4.9% w/v, less than about 4.8% w/v, less than about 4.7% w/v, less than about 4.6% w/v, less than about 4.5% w/v, less than about 4.4% w/v, less than about 4.3% w/v, less than about 4.2% w/v, less than about 4.1% w/v, less than about 4% w/v, less than about 3.9% w/v, less than about 3.8% w/v, less than about 3.7% w/v, less than about 3.6% w/v, less than about 3.5% w/v, less than about 3.4% w/v, less than about 3.3% w/v, less than about 3.2% w/v, less than about 3.1% w/v, less than about 3% w/v, less than about 2.9% w/v, less than about 2.8% w/v, less than about 2.7% w/v, less than about 2.6% w/v, less than about 2.5% w/v, less than about 2.4% w/v, less than about 2.3% w/v, less than about 2.2% w/v, less than about 2.1% w/v, less than about 2% w/v, less than about 1.9% w/v, less than about 1.8% w/v, less than about 1.7% w/v, less than about 1.6% w/v, less than about 1.5% w/v, less than about 1.4% w/v, less than about 1.3% w/v, less than about 1.2% w/v, less than about 1.1% w/v, less than about 1% w/v, less than about 0.8% w/v, less than about 0.8% w/v, less than about 0.7% w/v, less than about 0.6% w/v, less than about 0.5% w/v, less than about 0.4% w/v, less than about 0.3% w/v, less than about 0.2% w/v, or less than about 0.1% w/v.

In some embodiments, the aqueous-based composition contains one or more APIs in an amount that is greater than about 0.1% w/v, greater than about 0.2% w/v, greater than about 0.3% w/v, greater than about 0.4% w/v, greater than about 0.5% w/v, greater than about 0.6% w/v, greater than about 0.7% w/v, greater than about 0.8% w/v, greater than about 0.9% w/v, greater than about 1% w/v, greater than about 1.1% w/v, greater than about 1.2% w/v, greater than about 1.3% w/v, greater than about 1.4% w/v, greater than about 1.5% w/v, greater than about 1.6% w/v, greater than about 1.7% w/v, greater than about 1.8% w/v, greater than about 1.9% w/v, greater than about 2% w/v, greater than about 2.1% w/v, greater than about 2.2% w/v, greater than about 2.3% w/v, greater than about 2.4% w/v, greater than about 2.5% w/v, greater than about 2.6% w/v, greater than about 2.7% w/v, greater than about 2.8% w/v, greater than about 2.9% w/v, greater than about 3% w/v, greater than about 3.1% w/v, greater than about 3.2% w/v, greater than about 3.3% w/v, greater than about 3.4% w/v, greater than about 3.5% w/v, greater than about 3.6% w/v, greater than about 3.7% w/v, greater than about 3.8% w/v, greater than about 3.9% w/v, greater than about 4% w/v, greater than about 4.1% w/v, greater than about 4.2% w/v, greater than about 4.3% w/v, greater than about 4.4% w/v, greater than about 4.5% w/v, greater than about 4.6% w/v, greater than about 4.7% w/v, greater than about 4.8% w/v, greater than about 4.9% w/v, or greater than about 5% w/v.

In some embodiments, the aqueous-based composition contains one or more APIs in an amount that has a range of % w/v having an upper limit of about 5% w/v, 4% w/v, 3% w/v, 2% w/v, 1% w/v, 0.5% w/v, 0.4% w/v, 0.3% w/v, 0.2% w/v, or 0.1% w/v, and an independently selected lower limit of about 0.1% w/v, 0.2% w/v, 0.3% w/v, 0.4% w/v, 0.5% w/v, 1% w/v, 2% w/v, 3% w/v, 4% w/v, or 5% w/v, where the lower limit is less than the upper limit. In some embodiments, the aqueous-based composition contains one or more APIs in an amount of about 0.1% w/v, about 0.2% w/v, about 0.3% w/v, about 0.4% w/v, about 0.5% w/v, about 1% w/v, about 2% w/v, about 3% w/v, about 4% w/v, or about 5% w/v.

In some embodiments, the aqueous-based composition contains one or more APIs in an amount that ranges from about 0.2 mg/ml to about 100 mg/ml. As used herein, the term “mg/ml” refers to the total weight of the indicated component in milligrams as present in one milliliter of the final formulation of an individual aqueous-based composition of the present disclosure. In some embodiments, the aqueous-based composition contains one or more APIs in an amount that is less than about 100 mg/ml, less than about 95 mg/ml, less than about 90 mg/ml, less than about 85 mg/ml, less than about 80 mg/ml, less than about 75 mg/ml, less than about 70 mg/ml, less than about 65 mg/ml, less than about 60 mg/ml, less than about 55 mg/ml, less than about 50 mg/ml, less than about 45 mg/ml, less than about 40 mg/ml, less than about 35 mg/ml, less than about 30 mg/ml, less than about 25 mg/ml, less than about 20 mg/ml, less than about 15 mg/ml, less than about 10 mg/ml less than about 9 mg/ml, less than about 8 mg/ml, less than about 7 mg/ml, less than about 6 mg/ml, less than about 5 mg/ml, less than about 4 mg/ml, less than about 3 mg/ml, less than about 2 mg/ml, less than about 1 mg/ml, less than about 0.9 mg/ml, less than about 0.8 mg/ml, less than about 0.7 mg/ml, less than about 0.6 mg/ml, less than about 0.5 mg/ml, less than about 0.4 mg/ml, less than about 0.3 mg/ml, or less than about 0.2 mg/ml.

In some embodiments, the aqueous-based composition contains one or more APIs in an amount that is greater than about 0.2 mg/ml, greater than about 0.3 mg/ml, greater than about 0.4 mg/ml, greater than about 0.5 mg/ml, greater than about 0.6 mg/ml, greater than about 0.7 mg/ml, greater than about 0.8 mg/ml, greater than about 0.9 mg/ml, greater than about 1 mg/ml, greater than about 2 mg/ml, greater than about 3 mg/ml, greater than about 4 mg/ml, greater than about 5 mg/ml, greater than about 6 mg/ml, greater than about 7 mg/ml, greater than about 8 mg/ml, greater than about 9 mg/ml, greater than about 10 mg/ml, greater than about 15 mg/ml, greater than about 20 mg/ml, greater than about 25 mg/ml, greater than about 30 mg/ml, greater than about 35 mg/ml, greater than about 40 mg/ml, greater than about 45 mg/ml, greater than about 50 mg/ml, greater than about 55 mg/ml, greater than about 60 mg/ml, greater than about 65 mg/ml, greater than about 70 mg/ml, greater than about 75 mg/ml, greater than about 80 mg/ml, greater than about 85 mg/ml, greater than about 90 mg/ml, greater than about 95 mg/ml, or greater than about 100 mg/ml. In some embodiments, the aqueous-based composition contains one or more APIs in an amount that has a range of mg/ml having an upper limit of about 100 mg/ml, 80 mg/ml, 60 mg/ml, 40 mg/ml, 20 mg/ml, 10 mg/ml, 1 mg/ml, 0.8 mg/ml, 0.4 mg/ml, or 0.2 mg/ml and an independently selected lower limit of about 0.2 mg/ml, 0.4 mg/ml, 0.8 mg/ml, 1 mg/ml, 10 mg/ml, 20 mg/ml, 30 mg/ml, 40 mg/ml, 50 mg/ml, 60 mg/ml, 70 mg/ml, 80 mg/ml, 90 mg/ml, or 100 mg/ml, where the lower limit is less than the upper limit. In some embodiments, the aqueous-based composition contains one or more APIs in an amount of about 0.2 mg/ml, about 0.4 mg/ml, about 0.8 mg/ml, about 1 mg/ml, about 10 mg/ml, about 20 mg/ml, about 30 mg/ml, about 40 mg/ml, about 50 mg/ml, about 60 mg/ml, about 70 mg/ml, about 80 mg/ml, about 90 mg/ml, or about 100 mg/ml.

In some embodiments, the API is phenylephrine, or a pharmaceutically acceptable salt, ester, hydrate, or solvate thereof (e.g., phenylephrine HCl, phenylephrine bitartrate, etc.). In some embodiments, the API is dextromethorphan, or a pharmaceutically acceptable salt, ester, hydrate, or solvate thereof (e.g., dextromethorphan HBr, etc.). In some embodiments, the API is diphenhydramine, or a pharmaceutically acceptable salt, ester, hydrate, or solvate thereof (e.g., diphenhydramine HCl, etc.). In some embodiments, the API is chlorpheniramine, or a pharmaceutically acceptable salt, ester, hydrate, or solvate thereof (e.g., chlorpheniramine maleate, etc.). In some embodiments, the API is brompheniramine, or a pharmaceutically acceptable salt, ester, hydrate, or solvate thereof (e.g., brompheniramine maleate, etc.). In some embodiments, the API is loratadine, or a pharmaceutically acceptable salt, ester, hydrate, or solvate thereof. In some embodiments, the API is naproxen, or a pharmaceutically acceptable salt, ester, hydrate, or solvate thereof (e.g., naproxen sodium, etc.). In some embodiments, the API is ibuprofen, or a pharmaceutically acceptable salt, ester, hydrate, or solvate thereof (e.g., ibuprofen lysinate, etc.). In some embodiments, the API is acetaminophen (paracetamol), or a pharmaceutically acceptable salt, ester, hydrate, or solvate thereof. In some embodiments, the API is guaifenesin, or a pharmaceutically acceptable salt, ester, hydrate, or solvate thereof.

In some embodiments, the aqueous-based composition contains phenylephrine, or a pharmaceutically acceptable salt, ester, hydrate, or solvate thereof (e.g., phenylephrine HCl, phenylephrine bitartrate, etc.), in a pharmaceutically effective amount. In some embodiments, the pharmaceutically effective amount of phenylephrine ranges from 0.01% w/v to 0.05% w/v. In some embodiments, the aqueous-based composition contains phenylephrine, or a pharmaceutically acceptable salt, ester, hydrate, or solvate thereof, in an amount that is less than about 0.05% w/v, less than about 0.049% w/v, less than about 0.048% w/v, less than about 0.047% w/v, less than about 0.046% w/v, less than about 0.045% w/v, less than about 0.044% w/v, less than about 0.043% w/v, less than about 0.042% w/v, less than about 0.041% w/v, less than about 0.04% w/v, less than about 0.039% w/v, less than about 0.038% w/v, less than about 0.037% w/v, less than about 0.036% w/v, less than about 0.035% w/v, less than about 0.034% w/v, less than about 0.033% w/v, less than about 0.032% w/v, less than about 0.031% w/v, less than about 0.03% w/v, less than about 0.029% w/v, less than about 0.028% w/v, less than about 0.027% w/v, less than about 0.026% w/v, less than about 0.025% w/v, less than about 0.024% w/v, less than about 0.023% w/v, less than about 0.022% w/v, less than about 0.021% w/v, less than about 0.02% w/v, less than about 0.019% w/v, less than about 0.018% w/v, less than about 0.017% w/v, less than about 0.016% w/v, less than about 0.015% w/v, less than about 0.014% w/v, less than about 0.013% w/v, less than about 0.012% w/v, less than about 0.011% w/v, or less than about 0.01% w/v.

In some embodiments, the aqueous-based composition contains phenylephrine, or a pharmaceutically acceptable salt, ester, hydrate, or solvate thereof, in an amount that is greater than about 0.01% w/v, greater than about 0.011% w/v, greater than about 0.012% w/v, greater than about 0.013% w/v, greater than about 0.014% w/v, greater than about 0.015% w/v, greater than about 0.016% w/v, greater than about 0.017% w/v, greater than about 0.018% w/v, greater than about 0.019% w/v, greater than about 0.02% w/v, greater than about 0.021% w/v, greater than about 0.022% w/v, greater than about 0.023% w/v, greater than about 0.024% w/v, greater than about 0.025% w/v, greater than about 0.026% w/v, greater than about 0.027% w/v, greater than about 0.028% w/v, greater than about 0.029% w/v, greater than about 0.03% w/v, greater than about 0.031% w/v, greater than about 0.032% w/v, greater than about 0.033% w/v, greater than about 0.034% w/v, greater than about 0.035% w/v, greater than about 0.036% w/v, greater than about 0.037% w/v, greater than about 0.038% w/v, greater than about 0.039% w/v, greater than about 0.04% w/v, greater than about 0.041% w/v, greater than about 0.042% w/v, greater than about 0.043% w/v, greater than about 0.044% w/v, greater than about 0.045% w/v, greater than about 0.046% w/v, greater than about 0.047% w/v, greater than about 0.048% w/v, greater than about 0.049% w/v, or greater than about 0.05% w/v.

In some embodiments, the aqueous-based composition contains phenylephrine, or a pharmaceutically acceptable salt, ester, hydrate, or solvate thereof, in an amount that has a range of % w/v having an upper limit of about 0.05% w/v, 0.04% w/v, 0.03% w/v, 0.02% w/v, or 0.01% w/v, and an independently selected lower limit of about 0.01% w/v, 0.02% w/v, 0.03% w/v, 0.04% w/v, or 0.05% w/v, where the lower limit is less than the upper limit. In some embodiments, the aqueous-based composition contains phenylephrine, or a pharmaceutically acceptable salt, ester, hydrate, or solvate thereof, in an amount of about 0.01% w/v, about 0.02% w/v, about 0.03% w/v, about 0.04% w/v, or about 0.05% w/v.

In some embodiments, the aqueous-based composition contains phenylephrine, or a pharmaceutically acceptable salt, ester, hydrate, or solvate thereof (e.g., phenylephrine HCl, phenylephrine bitartrate, etc.), in a pharmaceutically effective amount. In some embodiments, the pharmaceutically effective amount of phenylephrine ranges from 0.3 mg/ml to 10 mg/ml. In some embodiments, the aqueous-based composition contains phenylephrine, or a pharmaceutically acceptable salt, ester, hydrate, or solvate thereof, in an amount that is less than about 10 mg/ml, less than about 9.9 mg/ml, less than about 9.8 mg/ml, less than about 9.7 mg/ml, less than about 9.6 mg/ml, less than about 9.5 mg/ml, less than about 9.4 mg/ml, less than about 9.3 mg/ml, less than about 9.2 mg/ml, less than about 9.1 mg/ml, less than about 9 mg/ml, less than about 8.9 mg/ml, less than about 8.8 mg/ml, less than about 8.7 mg/ml, less than about 8.6 mg/ml, less than about 8.5 mg/ml, less than about 8.4 mg/ml, less than about 8.3 mg/ml, less than about 8.2 mg/ml, less than about 8.1 mg/ml, less than about 8 mg/ml, less than about 7.9 mg/ml, less than about 7.8 mg/ml, less than about 7.7 mg/ml, less than about 7.6 mg/ml, less than about 7.5 mg/ml, less than about 7.4 mg/ml, less than about 7.3 mg/ml, less than about 7.2 mg/ml, less than about 7.1 mg/ml, less than about 7 mg/ml, less than about 6.9 mg/ml, less than about 6.8 mg/ml, less than about 6.7 mg/ml, less than about 6.6 mg/ml, less than about 6.5 mg/ml, less than about 6.4 mg/ml, less than about 6.3 mg/ml, less than about 6.2 mg/ml, less than about 6.1 mg/ml, less than about 6 mg/ml, less than about 5.9 mg/ml, less than about 5.8 mg/ml, less than about 5.7 mg/ml, less than about 5.6 mg/ml, less than about 5.5 mg/ml, less than about 5.4 mg/ml, less than about 5.3 mg/ml, less than about 5.2 mg/ml, less than about 5.1 mg/ml, less than about 5 mg/ml, less than about 4.9 mg/ml, less than about 4.8 mg/ml, less than about 4.7 mg/ml, less than about 4.6 mg/ml, less than about 4.5 mg/ml, less than about 4.4 mg/ml, less than about 4.3 mg/ml, less than about 4.2 mg/ml, less than about 4.1 mg/ml, less than about 4 mg/ml, less than about 3.9 mg/ml, less than about 3.8 mg/ml, less than about 3.7 mg/ml, less than about 3.6 mg/ml, less than about 3.5 mg/ml, less than about 3.4 mg/ml, less than about 3.3 mg/ml, less than about 3.2 mg/ml, less than about 3.1 mg/ml, less than about 3 mg/ml, less than about 2.9 mg/ml, less than about 2.8 mg/ml, less than about 2.7 mg/ml, less than about 2.6 mg/ml, less than about 2.5 mg/ml, less than about 2.4 mg/ml, less than about 2.3 mg/ml, less than about 2.2 mg/ml, less than about 2.1 mg/ml, less than about 2 mg/ml, less than about 1.9 mg/ml, less than about 1.8 mg/ml, less than about 1.7 mg/ml, less than about 1.6 mg/ml, less than about 1.5 mg/ml, less than about 1.4 mg/ml, less than about 1.3 mg/ml, less than about 1.2 mg/ml, less than about 1.1 mg/ml, less than about 1 mg/ml, less than about 0.9 mg/ml, less than about 0.8 mg/ml, less than about 0.7 mg/ml, less than about 0.6 mg/ml, less than about 0.5 mg/ml, less than about 0.4 mg/ml, or less than about 0.3 mg/ml.

In some embodiments, the aqueous-based composition contains phenylephrine, or a pharmaceutically acceptable salt, ester, hydrate, or solvate thereof, in an amount that is greater than about 0.3 mg/ml, greater than about 0.4 mg/ml, greater than about 0.5 mg/ml, greater than about 0.6 mg/ml, greater than about 0.7 mg/ml, greater than about 0.8 mg/ml, greater than about 0.9 mg/ml, greater than about 1 mg/ml, greater than about 1.1 mg/ml, greater than about 1.2 mg/ml, greater than about 1.3 mg/ml, greater than about 1.4 mg/ml, greater than about 1.5 mg/ml, greater than about 1.6 mg/ml, greater than about 1.7 mg/ml, greater than about 1.8 mg/ml, greater than about 1.9 mg/ml, greater than about 2 mg/ml, greater than about 2.1 mg/ml, greater than about 2.2 mg/ml, greater than about 2.3 mg/ml, greater than about 2.4 mg/ml, greater than about 2.5 mg/ml, greater than about 2.6 mg/ml, greater than about 2.7 mg/ml, greater than about 2.8 mg/ml, greater than about 2.9 mg/ml, greater than about 3 mg/ml, greater than about 3.1 mg/ml, greater than about 3.2 mg/ml, greater than about 3.3 mg/ml, greater than about 3.4 mg/ml, greater than about 3.5 mg/ml, greater than about 3.6 mg/ml, greater than about 3.7 mg/ml, greater than about 3.8 mg/ml, greater than about 3.9 mg/ml, greater than about 4 mg/ml, greater than about 4.1 mg/ml, greater than about 4.2 mg/ml, greater than about 4.3 mg/ml, greater than about 4.4 mg/ml, greater than about 4.5 mg/ml, greater than about 4.6 mg/ml, greater than about 4.7 mg/ml, greater than about 4.8 mg/ml, greater than about 4.9 mg/ml, greater than about 5 mg/ml, greater than about 5.1 mg/ml, greater than about 5.2 mg/ml, greater than about 5.3 mg/ml, greater than about 5.4 mg/ml, greater than about 5.5 mg/ml, greater than about 5.6 mg/ml, greater than about 5.7 mg/ml, greater than about 5.8 mg/ml, greater than about 5.9 mg/ml, greater than about 6 mg/ml, greater than about 6.1 mg/ml, greater than about 6.2 mg/ml, greater than about 6.3 mg/ml, greater than about 6.4 mg/ml, greater than about 6.5 mg/ml, greater than about 6.6 mg/ml, greater than about 6.7 mg/ml, greater than about 6.8 mg/ml, greater than about 6.9 mg/ml, greater than about 7 mg/ml, greater than about 7.1 mg/ml, greater than about 7.2 mg/ml, greater than about 7.3 mg/ml, greater than about 7.4 mg/ml, greater than about 7.5 mg/ml, greater than about 7.6 mg/ml, greater than about 7.7 mg/ml w/v, greater than about 7.8 mg/ml, greater than about 7.9 mg/ml, greater than about 8 mg/ml, greater than about 8.1 mg/ml, greater than about 8.2 mg/ml, greater than about 8.3 mg/ml, greater than about 8.4 mg/ml, greater than about 8.5 mg/ml, greater than about 8.6 mg/ml, greater than about 8.7 mg/ml, greater than about 8.8 mg/ml, greater than about 8.9 mg/ml, greater than about 9 mg/ml, greater than about 9.1 mg/ml, greater than about 9.2 mg/ml, greater than about 9.3 mg/ml, greater than about 9.4 mg/ml, greater than about 9.5 mg/ml, greater than about 9.6 mg/ml, greater than about 9.7 mg/ml, greater than about 9.8 mg/ml, greater than about 9.9 mg/ml, or greater than about 10 mg/ml.

In some embodiments, the aqueous-based composition contains phenylephrine, or a pharmaceutically acceptable salt, ester, hydrate, or solvate thereof, in an amount that has a range of mg/ml having an upper limit of about 10 mg/ml, 9 mg/ml, 8 mg/ml, 7 mg/ml, 6 mg/ml, 5 mg/ml, 4 mg/ml, 3 mg/ml, 2 mg/ml, 1.5 mg/ml, 1 mg/ml, or 0.5 mg/ml, and an independently selected lower limit of about 0.5 mg/ml, 1 mg/ml, 1.5 mg/ml, 2 mg/ml, 3 mg/ml, 4 mg/ml, 5 mg/ml, 6 mg/ml, 7 mg/ml, 8 mg/ml, 9 mg/ml, or 10 mg/ml, where the lower limit is less than the upper limit. In some embodiments, the aqueous-based composition contains phenylephrine, or a pharmaceutically acceptable salt, ester, hydrate, or solvate thereof, in an amount of about 0.5 mg/ml, about 1 mg/ml, about 1.5 mg/ml, about 2 mg/ml, about 3 mg/ml, about 4 mg/ml, about 5 mg/ml, about 6 mg/ml, about 7 mg/ml, about 8 mg/ml, about 9 mg/ml, or about 10 mg/ml. In some embodiments, the aqueous-based composition contains phenylephrine, or a pharmaceutically acceptable salt, ester, hydrate, or solvate thereof, in an amount of about 0.5 mg/ml. In some embodiments, the aqueous-based composition contains phenylephrine, or a pharmaceutically acceptable salt, ester, hydrate, or solvate thereof, in an amount of about 1.5 mg/ml.

In some embodiments, the aqueous-based composition contains dextromethorphan, or a pharmaceutically acceptable salt, ester, hydrate, or solvate thereof (e.g., dextromethorphan HBr, etc.), in a pharmaceutically effective amount. In some embodiments, the pharmaceutically effective amount of dextromethorphan ranges from 0.01% w/v to 0.10% w/v. In some embodiments, the aqueous-based composition contains dextromethorphan, or a pharmaceutically acceptable salt, ester, hydrate, or solvate thereof, in an amount that is less than about 0.1% w/v, less than about 0.099% w/v, less than about 0.098% w/v, less than about 0.097% w/v, less than about 0.096% w/v, less than about 0.095% w/v, less than about 0.094% w/v, less than about 0.093% w/v, less than about 0.092% w/v, less than about 0.091% w/v, less than about 0.09% w/v, less than about 0.089% w/v, less than about 0.088% w/v, less than about 0.087% w/v, less than about 0.086% w/v, less than about 0.085% w/v, less than about 0.084% w/v, less than about 0.083% w/v, less than about 0.082% w/v, less than about 0.081% w/v, less than about 0.08% w/v, less than about 0.079% w/v, less than about 0.078% w/v, less than about 0.077% w/v, less than about 0.076% w/v, less than about 0.075% w/v, less than about 0.074% w/v, less than about 0.073% w/v, less than about 0.072% w/v, less than about 0.071% w/v, less than about 0.07% w/v, less than about 0.069% w/v, less than about 0.068% w/v, less than about 0.067% w/v, less than about 0.066% w/v, less than about 0.065% w/v, less than about 0.064% w/v, less than about 0.063% w/v, less than about 0.062% w/v, less than about 0.061% w/v, less than about 0.06% w/v, less than about 0.059% w/v, less than about 0.058% w/v, less than about 0.057% w/v, less than about 0.056% w/v, less than about 0.055% w/v, less than about 0.054% w/v, less than about 0.053% w/v, less than about 0.052% w/v, less than about 0.051% w/v, 0.05% w/v, less than about 0.049% w/v, less than about 0.048% w/v, less than about 0.047% w/v, less than about 0.046% w/v, less than about 0.045% w/v, less than about 0.044% w/v, less than about 0.043% w/v, less than about 0.042% w/v, less than about 0.041% w/v, less than about 0.04% w/v, less than about 0.039% w/v, less than about 0.038% w/v, less than about 0.037% w/v, less than about 0.036% w/v, less than about 0.035% w/v, less than about 0.034% w/v, less than about 0.033% w/v, less than about 0.032% w/v, less than about 0.031% w/v, less than about 0.03% w/v, less than about 0.029% w/v, less than about 0.028% w/v, less than about 0.027% w/v, less than about 0.026% w/v, less than about 0.025% w/v, less than about 0.024% w/v, less than about 0.023% w/v, less than about 0.022% w/v, less than about 0.021% w/v, less than about 0.02% w/v, less than about 0.019% w/v, less than about 0.018% w/v, less than about 0.017% w/v, less than about 0.016% w/v, less than about 0.015% w/v, less than about 0.014% w/v, less than about 0.013% w/v, less than about 0.012% w/v, less than about 0.011% w/v, or less than about 0.01% w/v.

In some embodiments, the aqueous-based composition contains dextromethorphan, or a pharmaceutically acceptable salt, ester, hydrate, or solvate thereof, in an amount that is greater than about 0.01% w/v, greater than about 0.011% w/v, greater than about 0.012% w/v, greater than about 0.013% w/v, greater than about 0.014% w/v, greater than about 0.015% w/v, greater than about 0.016% w/v, greater than about 0.017% w/v, greater than about 0.018% w/v, greater than about 0.019% w/v, greater than about 0.02% w/v, greater than about 0.021% w/v, greater than about 0.022% w/v, greater than about 0.023% w/v, greater than about 0.024% w/v, greater than about 0.025% w/v, greater than about 0.026% w/v, greater than about 0.027% w/v, greater than about 0.028% w/v, greater than about 0.029% w/v, greater than about 0.03% w/v, greater than about 0.031% w/v, greater than about 0.032% w/v, greater than about 0.033% w/v, greater than about 0.034% w/v, greater than about 0.035% w/v, greater than about 0.036% w/v, greater than about 0.037% w/v, greater than about 0.038% w/v, greater than about 0.039% w/v, greater than about 0.04% w/v, greater than about 0.041% w/v, greater than about 0.042% w/v, greater than about 0.043% w/v, greater than about 0.044% w/v, greater than about 0.045% w/v, greater than about 0.046% w/v, greater than about 0.047% w/v, greater than about 0.048% w/v, greater than about 0.049% w/v, greater than about 0.05% w/v, greater than about 0.051% w/v, greater than about 0.052% w/v, greater than about 0.053% w/v, greater than about 0.054% w/v, greater than about 0.055% w/v, greater than about 0.056% w/v, greater than about 0.057% w/v, greater than about 0.058% w/v, greater than about 0.059% w/v, greater than about 0.06% w/v, greater than about 0.061% w/v, greater than about 0.062% w/v, greater than about 0.063% w/v, greater than about 0.064% w/v, greater than about 0.065% w/v, greater than about 0.066% w/v, greater than about 0.067% w/v, greater than about 0.068% w/v, greater than about 0.069% w/v, greater than about 0.07% w/v, greater than about 0.071% w/v, greater than about 0.072% w/v, greater than about 0.073% w/v, greater than about 0.074% w/v, greater than about 0.075% w/v, greater than about 0.076% w/v, greater than about 0.077% w/v, greater than about 0.078% w/v, greater than about 0.079% w/v, greater than about 0.08% w/v, greater than about 0.081% w/v, greater than about 0.082% w/v, greater than about 0.083% w/v, greater than about 0.084% w/v, greater than about 0.085% w/v, greater than about 0.086% w/v, greater than about 0.087% w/v, greater than about 0.088% w/v, greater than about 0.089% w/v, greater than about 0.09% w/v, greater than about 0.091% w/v, greater than about 0.092% w/v, greater than about 0.093% w/v, greater than about 0.094% w/v, greater than about 0.095% w/v, greater than about 0.096% w/v, greater than about 0.097% w/v, greater than about 0.098% w/v, greater than about 0.099% w/v, or greater than about 0.1% w/v.

In some embodiments, the aqueous-based composition contains dextromethorphan, or a pharmaceutically acceptable salt, ester, hydrate, or solvate thereof, in an amount that has a range of % w/v having an upper limit of about 0.1% w/v, 0.09% w/v, 0.08% w/v, 0.07% w/v, 0.06% w/v, 0.05% w/v, 0.04% w/v, 0.03% w/v, 0.02% w/v, or 0.01% w/v, and an independently selected lower limit of about 0.01% w/v, 0.02% w/v, 0.03% w/v, 0.04% w/v, 0.05% w/v, 0.06% w/v, 0.07% w/v, 0.08% w/v, 0.09% w/v, or 0.1% w/v, where the lower limit is less than the upper limit. In some embodiments, the aqueous-based composition contains dextromethorphan, or a pharmaceutically acceptable salt, ester, hydrate, or solvate thereof, in an amount of about 0.01% w/v, about 0.02% w/v, about 0.03% w/v, about 0.04% w/v, about 0.05% w/v, about 0.06% w/v, about 0.07% w/v, about 0.08% w/v, about 0.09% w/v, or about 0.1% w/v.

In some embodiments, the aqueous-based composition contains dextromethorphan, or a pharmaceutically acceptable salt, ester, hydrate, or solvate thereof (e.g., dextromethorphan HBr, etc.), in a pharmaceutically effective amount. In some embodiments, the pharmaceutically effective amount of dextromethorphan ranges from 1.3 mg/ml to 3 mg/ml. In some embodiments, the aqueous-based composition contains dextromethorphan, or a pharmaceutically acceptable salt, ester, hydrate, or solvate thereof, in an amount that is less than about 3 mg/ml, less than about 2.99 mg/ml, less than about 2.98 mg/ml, less than about 2.97 mg/ml, less than about 2.96 mg/ml, less than about 2.95 mg/ml, less than about 2.94 mg/ml, less than about 2.93 mg/ml, less than about 2.92 mg/ml, less than about 2.91 mg/ml, less than about 2.9 mg/ml, less than about 2.89 mg/ml, less than about 2.88 mg/ml, less than about 2.87 mg/ml, less than about 2.86 mg/ml, less than about 2.85 mg/ml, less than about 2.84 mg/ml, less than about 2.83 mg/ml, less than about 2.82 mg/ml, less than about 2.81 mg/ml, less than about 2.8 mg/ml, less than about 2.79 mg/ml, less than about 2.78 mg/ml, less than about 2.77 mg/ml, less than about 2.76 mg/ml, less than about 2.75 mg/ml, less than about 2.74 mg/ml, less than about 2.73 mg/ml, less than about 2.72 mg/ml, less than about 2.71 mg/ml, less than about 2.7 mg/ml, less than about 2.69 mg/ml, less than about 2.68 mg/ml, less than about 2.67 mg/ml, less than about 2.66 mg/ml, less than about 2.65 mg/ml, less than about 2.64 mg/ml, less than about 2.63 mg/ml, less than about 2.62 mg/ml, less than about 2.61 mg/ml, less than about 2.6 mg/ml, less than about 2.59 mg/ml, less than about 2.58 mg/ml, less than about 2.57 mg/ml, less than about 2.56 mg/ml, less than about 2.55 mg/ml, less than about 2.54 mg/ml, less than about 2.53 mg/ml, less than about 2.52 mg/ml, less than about 2.51 mg/ml, less than about 2.5 mg/ml, less than about 2.49 mg/ml, less than about 2.48 mg/ml, less than about 2.47 mg/ml, less than about 2.46 mg/ml, less than about 2.45 mg/ml, less than about 2.44 mg/ml, less than about 2.43 mg/ml, less than about 2.42 mg/ml, less than about 2.41 mg/ml, less than about 2.4 mg/ml, less than about 2.39 mg/ml, less than about 2.38 mg/ml, less than about 2.37 mg/ml, less than about 2.36 mg/ml, less than about 2.35 mg/ml, less than about 2.34 mg/ml, less than about 2.33 mg/ml, less than about 2.32 mg/ml, less than about 2.31 mg/ml, less than about 2.3 mg/ml, less than about 2.29 mg/ml, less than about 2.28 mg/ml, less than about 2.27 mg/ml, less than about 2.26 mg/ml, less than about 2.25 mg/ml, less than about 2.24 mg/ml, less than about 2.23 mg/ml, less than about 2.22 mg/ml, less than about 2.21 mg/ml, less than about 2.2 mg/ml, less than about 2.19 mg/ml, less than about 2.18 mg/ml, less than about 2.17 mg/ml, less than about 2.16 mg/ml, less than about 2.15 mg/ml, less than about 2.14 mg/ml, less than about 2.13 mg/ml, less than about 2.12 mg/ml, less than about 2.11 mg/ml, less than about 2 mg/ml, less than about 1.99 mg/ml, less than about 1.98 mg/ml, less than about 1.97 mg/ml, less than about 1.96 mg/ml, less than about 1.95 mg/ml, less than about 1.94 mg/ml, less than about 1.93 mg/ml, less than about 1.92 mg/ml, less than about 1.91 mg/ml, less than about 1.9 mg/ml, less than about 1.89 mg/ml, less than about 1.88 mg/ml, less than about 1.87 mg/ml, less than about 1.86 mg/ml, less than about 1.85 mg/ml, less than about 1.84 mg/ml, less than about 1.83 mg/ml, less than about 1.82 mg/ml, less than about 1.81 mg/ml, less than about 1.8 mg/ml, less than about 1.79 mg/ml, less than about 1.78 mg/ml, less than about 1.77 mg/ml, less than about 1.76 mg/ml, less than about 1.75 mg/ml, less than about 1.74 mg/ml, less than about 1.73 mg/ml, less than about 1.72 mg/ml, less than about 1.71 mg/ml, less than about 1.7 mg/ml, less than about 1.69 mg/ml, less than about 1.68 mg/ml, less than about 1.67 mg/ml, less than about 1.66 mg/ml, less than about 1.65 mg/ml, less than about 1.64 mg/ml, less than about 1.63 mg/ml, less than about 1.62 mg/ml, less than about 1.61 mg/ml, less than about 1.6 mg/ml, less than about 1.59 mg/ml, less than about 1.58 mg/ml, less than about 1.57 mg/ml, less than about 1.56 mg/ml, less than about 1.55 mg/ml, less than about 1.54 mg/ml, less than about 1.53 mg/ml, less than about 1.52 mg/ml, less than about 1.51 mg/ml, less than about 1.5 mg/ml, less than about 1.49 mg/ml, less than about 1.48 mg/ml, less than about 1.47 mg/ml, less than about 1.46 mg/ml, less than about 1.45 mg/ml, less than about 1.44 mg/ml, less than about 1.43 mg/ml, less than about 1.42 mg/ml, less than about 1.41 mg/ml, less than about 1.4 mg/ml, less than about 1.39 mg/ml, less than about 1.38 mg/ml, less than about 1.37 mg/ml, less than about 1.36 mg/ml, less than about 1.35 mg/ml, less than about 1.34 mg/ml, less than about 1.33 mg/ml, less than about 1.32 mg/ml, less than about 1.31 mg/ml, or less than about 1.3 mg/ml.

In some embodiments, the aqueous-based composition contains dextromethorphan, or a pharmaceutically acceptable salt, ester, hydrate, or solvate thereof, in an amount that is greater than about 1.3 mg/ml, greater than about 1.31 mg/ml, greater than about 1.32 mg/ml, greater than about 1.33 mg/ml, greater than about 1.34 mg/ml, greater than about 1.35 mg/ml, greater than about 1.36 mg/ml, greater than about 1.37 mg/ml, greater than about 1.38 mg/ml, greater than about 1.39 mg/ml, greater than about 1.4 mg/ml, greater than about 1.41 mg/ml, greater than about 1.42 mg/ml, greater than about 1.43 mg/ml, greater than about 1.44 mg/ml, greater than about 1.45 mg/ml, greater than about 1.46 mg/ml, greater than about 1.47 mg/ml, greater than about 1.48 mg/ml, greater than about 1.49 mg/ml, greater than about 1.5 mg/ml, greater than about 1.51 mg/ml, greater than about 1.52 mg/ml, greater than about 1.53 mg/ml, greater than about 1.54 mg/ml, greater than about 1.55 mg/ml, greater than about 1.56 mg/ml, greater than about 1.57 mg/ml, greater than about 1.58 mg/ml, greater than about 1.59 mg/ml, greater than about 1.6 mg/ml, greater than about 1.61 mg/ml, greater than about 1.62 mg/ml, greater than about 1.63 mg/ml, greater than about 1.64 mg/ml, greater than about 1.65 mg/ml, greater than about 1.66 mg/ml, greater than about 1.67 mg/ml, greater than about 1.68 mg/ml, greater than about 1.69 mg/ml, greater than about 1.7 mg/ml, greater than about 1.71 mg/ml, greater than about 1.72 mg/ml, greater than about 1.73 mg/ml, greater than about 1.74 mg/ml, greater than about 1.75 mg/ml, greater than about 1.76 mg/ml, greater than about 1.77 mg/ml w/v, greater than about 1.78 mg/ml, greater than about 1.79 mg/ml, greater than about 1.8 mg/ml, greater than about 1.81 mg/ml, greater than about 1.82 mg/ml, greater than about 1.83 mg/ml, greater than about 1.84 mg/ml, greater than about 1.85 mg/ml, greater than about 1.86 mg/ml, greater than about 1.87 mg/ml, greater than about 1.88 mg/ml, greater than about 1.89 mg/ml, greater than about 1.9 mg/ml, greater than about 1.91 mg/ml, greater than about 1.92 mg/ml, greater than about 1.93 mg/ml, greater than about 1.94 mg/ml, greater than about 1.95 mg/ml, greater than about 1.96 mg/ml, greater than about 1.97 mg/ml, greater than about 1.98 mg/ml, greater than about 1.99 mg/ml, greater than about 2 mg/ml, greater than about 2.1 mg/ml, greater than about 2.11 mg/ml, greater than about 2.12 mg/ml, greater than about 2.13 mg/ml, greater than about 2.14 mg/ml, greater than about 2.15 mg/ml, greater than about 2.16 mg/ml, greater than about 2.17 mg/ml, greater than about 2.18 mg/ml, greater than about 2.19 mg/ml, greater than about 2.2 mg/ml, greater than about 2.21 mg/ml, greater than about 2.22 mg/ml, greater than about 2.23 mg/ml, greater than about 2.24 mg/ml, greater than about 2.25 mg/ml, greater than about 2.26 mg/ml, greater than about 2.27 mg/ml, greater than about 2.28 mg/ml, greater than about 2.29 mg/ml, greater than about 2.3 mg/ml, greater than about 2.31 mg/ml, greater than about 2.32 mg/ml, greater than about 2.33 mg/ml, greater than about 2.34 mg/ml, greater than about 2.35 mg/ml, greater than about 2.36 mg/ml, greater than about 2.37 mg/ml, greater than about 2.38 mg/ml, greater than about 2.39 mg/ml, greater than about 2.4 mg/ml, greater than about 2.41 mg/ml, greater than about 2.42 mg/ml, greater than about 2.43 mg/ml, greater than about 2.44 mg/ml, greater than about 2.45 mg/ml, greater than about 2.46 mg/ml, greater than about 2.47 mg/ml, greater than about 2.48 mg/ml, greater than about 2.49 mg/ml, greater than about 2.5 mg/ml, greater than about 2.51 mg/ml, greater than about 2.52 mg/ml, greater than about 2.53 mg/ml, greater than about 2.54 mg/ml, greater than about 2.55 mg/ml, greater than about 2.56 mg/ml, greater than about 2.57 mg/ml, greater than about 2.58 mg/ml, greater than about 2.59 mg/ml, greater than about 2.6 mg/ml, greater than about 2.61 mg/ml, greater than about 2.62 mg/ml, greater than about 2.63 mg/ml, greater than about 2.64 mg/ml, greater than about 2.65 mg/ml, greater than about 2.66 mg/ml, greater than about 2.67 mg/ml, greater than about 2.68 mg/ml, greater than about 2.69 mg/ml, greater than about 2.7 mg/ml, greater than about 2.71 mg/ml, greater than about 2.72 mg/ml, greater than about 2.73 mg/ml, greater than about 2.74 mg/ml, greater than about 2.75 mg/ml, greater than about 2.76 mg/ml, greater than about 2.77 mg/ml w/v, greater than about 2.78 mg/ml, greater than about 2.79 mg/ml, greater than about 2.8 mg/ml, greater than about 2.81 mg/ml, greater than about 2.82 mg/ml, greater than about 2.83 mg/ml, greater than about 2.84 mg/ml, greater than about 2.85 mg/ml, greater than about 2.86 mg/ml, greater than about 2.87 mg/ml, greater than about 2.88 mg/ml, greater than about 2.89 mg/ml, greater than about 2.9 mg/ml, greater than about 2.91 mg/ml, greater than about 2.92 mg/ml, greater than about 2.93 mg/ml, greater than about 2.94 mg/ml, greater than about 2.95 mg/ml, greater than about 2.96 mg/ml, greater than about 2.97 mg/ml, greater than about 2.98 mg/ml, greater than about 2.99 mg/ml, or greater than about 3 mg/ml.

In some embodiments, the aqueous-based composition contains dextromethorphan, or a pharmaceutically acceptable salt, ester, hydrate, or solvate thereof, in an amount that has a range of mg/ml having an upper limit of about 3 mg/ml, 2.9 mg/ml, 2.8 mg/ml, 2.7 mg/ml, 2.6 mg/ml, 2.5 mg/ml, 2.4 mg/ml, 2.3 mg/ml, 2.2 mg/ml, 2.1 mg/ml, 2 mg/ml, 1.9 mg/ml, 1.8 mg/ml, 1.7 mg/ml, 1.6 mg/ml, 1.5 mg/ml, 1.4 mg/ml, or 1.3 mg/ml and an independently selected lower limit of about 1.3 mg/ml, 1.4 mg/ml, 1.5 mg/ml, 1.6 mg/ml, 1.7 mg/ml, 1.8 mg/ml, 1.9 mg/ml, 2 mg/ml, 2.1 mg/ml, 2.2 mg/ml, 2.3 mg/ml, 2.4 mg/ml, 2.5 mg/ml, 2.6 mg/ml, 2.7 mg/ml, 2.8 mg/ml, 2.9 mg/ml, or 3 mg/ml, where the lower limit is less than the upper limit. In some embodiments, the aqueous-based composition contains dextromethorphan, or a pharmaceutically acceptable salt, ester, hydrate, or solvate thereof, in an amount of about 1.3 mg/ml, 1.4 mg/ml, about 1.5 mg/ml, about 1.6 mg/ml, about 1.7 mg/ml, about 1.8 mg/ml, about 1.9 mg/ml, about 2 mg/ml, about 2.1 mg/ml, about 2.2 mg/ml, about 2.3 mg/ml, about 2.4 mg/ml, about 2.5 mg/ml, about 2.6 mg/ml, about 2.7 mg/ml, about 2.8 mg/ml, about 2.9 mg/ml, or about 3 mg/ml. In some embodiments, the aqueous-based composition contains dextromethorphan, or a pharmaceutically acceptable salt, ester, hydrate, or solvate thereof, in an amount of about 1.5 mg/ml. In some embodiments, the aqueous-based composition contains dextromethorphan, or a pharmaceutically acceptable salt, ester, hydrate, or solvate thereof, in an amount of about 2 mg/ml.

In some embodiments, the aqueous-based composition contains diphenhydramine, or a pharmaceutically acceptable salt, ester, hydrate, or solvate thereof (e.g., diphenhydramine HCl, etc.), in a pharmaceutically effective amount. In some embodiments, the pharmaceutically effective amount of diphenhydramine ranges from 0.01% w/v to 0.10% w/v. In some embodiments, the aqueous-based composition contains diphenhydramine, or a pharmaceutically acceptable salt, ester, hydrate, or solvate thereof, in an amount that is less than about 0.1% w/v, less than about 0.099% w/v, less than about 0.098% w/v, less than about 0.097% w/v, less than about 0.096% w/v, less than about 0.095% w/v, less than about 0.094% w/v, less than about 0.093% w/v, less than about 0.092% w/v, less than about 0.091% w/v, less than about 0.09% w/v, less than about 0.089% w/v, less than about 0.088% w/v, less than about 0.087% w/v, less than about 0.086% w/v, less than about 0.085% w/v, less than about 0.084% w/v, less than about 0.083% w/v, less than about 0.082% w/v, less than about 0.081% w/v, less than about 0.08% w/v, less than about 0.079% w/v, less than about 0.078% w/v, less than about 0.077% w/v, less than about 0.076% w/v, less than about 0.075% w/v, less than about 0.074% w/v, less than about 0.073% w/v, less than about 0.072% w/v, less than about 0.071% w/v, less than about 0.07% w/v, less than about 0.069% w/v, less than about 0.068% w/v, less than about 0.067% w/v, less than about 0.066% w/v, less than about 0.065% w/v, less than about 0.064% w/v, less than about 0.063% w/v, less than about 0.062% w/v, less than about 0.061% w/v, less than about 0.06% w/v, less than about 0.059% w/v, less than about 0.058% w/v, less than about 0.057% w/v, less than about 0.056% w/v, less than about 0.055% w/v, less than about 0.054% w/v, less than about 0.053% w/v, less than about 0.052% w/v, less than about 0.051% w/v, 0.05% w/v, less than about 0.049% w/v, less than about 0.048% w/v, less than about 0.047% w/v, less than about 0.046% w/v, less than about 0.045% w/v, less than about 0.044% w/v, less than about 0.043% w/v, less than about 0.042% w/v, less than about 0.041% w/v, less than about 0.04% w/v, less than about 0.039% w/v, less than about 0.038% w/v, less than about 0.037% w/v, less than about 0.036% w/v, less than about 0.035% w/v, less than about 0.034% w/v, less than about 0.033% w/v, less than about 0.032% w/v, less than about 0.031% w/v, less than about 0.03% w/v, less than about 0.029% w/v, less than about 0.028% w/v, less than about 0.027% w/v, less than about 0.026% w/v, less than about 0.025% w/v, less than about 0.024% w/v, less than about 0.023% w/v, less than about 0.022% w/v, less than about 0.021% w/v, less than about 0.02% w/v, less than about 0.019% w/v, less than about 0.018% w/v, less than about 0.017% w/v, less than about 0.016% w/v, less than about 0.015% w/v, less than about 0.014% w/v, less than about 0.013% w/v, less than about 0.012% w/v, less than about 0.011% w/v, or less than about 0.01% w/v.

In some embodiments, the aqueous-based composition contains diphenhydramine, or a pharmaceutically acceptable salt, ester, hydrate, or solvate thereof, in an amount that is greater than about 0.01% w/v, greater than about 0.011% w/v, greater than about 0.012% w/v, greater than about 0.013% w/v, greater than about 0.014% w/v, greater than about 0.015% w/v, greater than about 0.016% w/v, greater than about 0.017% w/v, greater than about 0.018% w/v, greater than about 0.019% w/v, greater than about 0.02% w/v, greater than about 0.021% w/v, greater than about 0.022% w/v, greater than about 0.023% w/v, greater than about 0.024% w/v, greater than about 0.025% w/v, greater than about 0.026% w/v, greater than about 0.027% w/v, greater than about 0.028% w/v, greater than about 0.029% w/v, greater than about 0.03% w/v, greater than about 0.031% w/v, greater than about 0.032% w/v, greater than about 0.033% w/v, greater than about 0.034% w/v, greater than about 0.035% w/v, greater than about 0.036% w/v, greater than about 0.037% w/v, greater than about 0.038% w/v, greater than about 0.039% w/v, greater than about 0.04% w/v, greater than about 0.041% w/v, greater than about 0.042% w/v, greater than about 0.043% w/v, greater than about 0.044% w/v, greater than about 0.045% w/v, greater than about 0.046% w/v, greater than about 0.047% w/v, greater than about 0.048% w/v, greater than about 0.049% w/v, greater than about 0.05% w/v, greater than about 0.051% w/v, greater than about 0.052% w/v, greater than about 0.053% w/v, greater than about 0.054% w/v, greater than about 0.055% w/v, greater than about 0.056% w/v, greater than about 0.057% w/v, greater than about 0.058% w/v, greater than about 0.059% w/v, greater than about 0.06% w/v, greater than about 0.061% w/v, greater than about 0.062% w/v, greater than about 0.063% w/v, greater than about 0.064% w/v, greater than about 0.065% w/v, greater than about 0.066% w/v, greater than about 0.067% w/v, greater than about 0.068% w/v, greater than about 0.069% w/v, greater than about 0.07% w/v, greater than about 0.071% w/v, greater than about 0.072% w/v, greater than about 0.073% w/v, greater than about 0.074% w/v, greater than about 0.075% w/v, greater than about 0.076% w/v, greater than about 0.077% w/v, greater than about 0.078% w/v, greater than about 0.079% w/v, greater than about 0.08% w/v, greater than about 0.081% w/v, greater than about 0.082% w/v, greater than about 0.083% w/v, greater than about 0.084% w/v, greater than about 0.085% w/v, greater than about 0.086% w/v, greater than about 0.087% w/v, greater than about 0.088% w/v, greater than about 0.089% w/v, greater than about 0.09% w/v, greater than about 0.091% w/v, greater than about 0.092% w/v, greater than about 0.093% w/v, greater than about 0.094% w/v, greater than about 0.095% w/v, greater than about 0.096% w/v, greater than about 0.097% w/v, greater than about 0.098% w/v, greater than about 0.099% w/v, or greater than about 0.1% w/v.

In some embodiments, the aqueous-based composition contains diphenhydramine, or a pharmaceutically acceptable salt, ester, hydrate, or solvate thereof, in an amount that has a range of % w/v having an upper limit of about 0.1% w/v, 0.09% w/v, 0.08% w/v, 0.07% w/v, 0.06% w/v, 0.05% w/v, 0.04% w/v, 0.03% w/v, 0.02% w/v, or 0.01% w/v, and an independently selected lower limit of about 0.01% w/v, 0.02% w/v, 0.03% w/v, 0.04% w/v, 0.05% w/v, 0.06% w/v, 0.07% w/v, 0.08% w/v, 0.09% w/v, or 0.1% w/v, where the lower limit is less than the upper limit. In some embodiments, the aqueous-based composition contains diphenhydramine, or a pharmaceutically acceptable salt, ester, hydrate, or solvate thereof, in an amount of about 0.01% w/v, about 0.02% w/v, about 0.03% w/v, about 0.04% w/v, about 0.05% w/v, about 0.06% w/v, about 0.07% w/v, about 0.08% w/v, about 0.09% w/v, or about 0.1% w/v.

In some embodiments, the aqueous-based composition contains diphenhydramine, or a pharmaceutically acceptable salt, ester, hydrate, or solvate thereof (e.g., diphenhydramine HCl, etc.), in a pharmaceutically effective amount. In some embodiments, the pharmaceutically effective amount of diphenhydramine ranges from 1.5 mg/ml to 50 mg/ml. In some embodiments, the aqueous-based composition contains diphenhydramine, or a pharmaceutically acceptable salt, ester, hydrate, or solvate thereof, in an amount that is less than about 50 mg/ml, less than about 45 mg/ml, less than about 40 mg/ml, less than about 35 mg/ml, less than about 30 mg/ml, less than about 25 mg/ml, less than about 20 mg/ml, less than about 19 mg/ml, less than about 18 mg/ml, less than about 17 mg/ml, less than about 16 mg/ml, less than about 15 mg/ml, less than about 14 mg/ml, less than about 13 mg/ml, less than about 12 mg/ml, less than about 11 mg/ml, less than about 10 mg/ml, less than about 9.75 mg/ml, less than about 9.5 mg/ml, less than about 9.25 mg/ml, less than about 9 mg/ml, less than about 8.75 mg/ml, less than about 8.5 mg/ml, less than about 8.25 mg/ml, less than about 8 mg/ml, less than about 7.75 mg/ml, less than about 7.5 mg/ml, less than about 7.25 mg/ml, less than about 7 mg/ml, less than about 6.75 mg/ml, less than about 6.5 mg/ml, less than about 6.25 mg/ml, less than about 6 mg/ml, less than about 5.75 mg/ml, less than about 5.5 mg/ml, less than about 5.25 mg/ml, less than about 5 mg/ml, less than about 4.75 mg/ml, less than about 4.5 mg/ml, less than about 4.25 mg/ml, less than about 4 mg/ml, less than about 3.75 mg/ml, less than about 3.5 mg/ml, less than about 3.25 mg/ml, less than about 3 mg/ml, less than about 2.75 mg/ml, less than about 2.5 mg/ml, less than about 2.25 mg/ml, less than about 2 mg/ml, less than about 1.75 mg/ml, or less than about 1.5 mg/ml.

In some embodiments, the aqueous-based composition contains diphenhydramine, or a pharmaceutically acceptable salt, ester, hydrate, or solvate thereof, in an amount that is greater than about 1.5 mg/ml, greater than about 1.75 mg/ml, greater than about 2 mg/ml, greater than about 2.25 mg/ml, greater than about 2.5 mg/ml, greater than about 2.75 mg/ml, greater than about 3 mg/ml, greater than about 3.25 mg/ml, greater than about 3.5 mg/ml, greater than about 3.75 mg/ml, greater than about 4 mg/ml, greater than about 4.25 mg/ml, greater than about 4.5 mg/ml, greater than about 4.75 mg/ml, greater than about 5 mg/ml, greater than about 5.25 mg/ml, greater than about 5.5 mg/ml, greater than about 5.75 mg/ml, greater than about 6 mg/ml, greater than about 6.25 mg/ml, greater than about 6.5 mg/ml, greater than about 6.75 mg/ml, greater than about 7 mg/ml, greater than about 7.25 mg/ml, greater than about 7.5 mg/ml, greater than about 7.75 mg/ml, greater than about 8 mg/ml, greater than about 8.25 mg/ml, greater than about 8.5 mg/ml, greater than about 8.75 mg/ml, greater than about 9 mg/ml, greater than about 9.25 mg/ml, greater than about 9.5 mg/ml, greater than about 9.75 mg/ml, greater than about 10 mg/ml, greater than about 11 mg/ml, greater than about 12 mg/ml, greater than about 13 mg/ml, greater than about 14 mg/ml, greater than about 15 mg/ml, greater than about 16 mg/ml, greater than about 17 mg/ml, greater than about 18 mg/ml, greater than about 19 mg/ml, greater than about 20 mg/ml, greater than about 25 mg/ml, greater than about 30 mg/ml, greater than about 35 mg/ml, greater than about 40 mg/ml, greater than about 45 mg/ml, or greater than about 50 mg/ml.

In some embodiments, the aqueous-based composition contains diphenhydramine, or a pharmaceutically acceptable salt, ester, hydrate, or solvate thereof, in an amount that has a range of mg/ml having an upper limit of about 50 mg/ml, 40 mg/ml, 30 mg/ml, 20 mg/ml, 15 mg/ml, 10 mg/ml, 6.25 mg/ml, 5 mg/ml, 2.5 mg/ml, 2 mg/ml, or 1.5 mg/ml and an independently selected lower limit of about 1.5 mg/ml, 2 mg/ml, 2.5 mg/ml, 5 mg/ml, 6.25 mg/ml, 10 mg/ml, 15 mg/ml, 20 mg/ml, 30 mg/ml, 40 mg/ml, or 50 mg/ml, where the lower limit is less than the upper limit. In some embodiments, the aqueous-based composition contains diphenhydramine, or a pharmaceutically acceptable salt, ester, hydrate, or solvate thereof, in an amount of about 1.5 mg/ml, about 2 mg/ml, about 2.5 mg/ml, about 5 mg/ml, about 6.25 mg/ml, about 10 mg/ml, about 15 mg/ml, about 20 mg/ml, about 30 mg/ml, about 40 mg/ml, or about 50 mg/ml. In some embodiments, the aqueous-based composition contains diphenhydramine, or a pharmaceutically acceptable salt, ester, hydrate, or solvate thereof, in an amount of about 2.5 mg/ml. In some embodiments, the aqueous-based composition contains diphenhydramine, or a pharmaceutically acceptable salt, ester, hydrate, or solvate thereof, in an amount of about 6.25 mg/ml.

In some embodiments, the aqueous-based composition contains chlorpheniramine, or a pharmaceutically acceptable salt, ester, hydrate, or solvate thereof (e.g., chlorpheniramine maleate, etc.), in a pharmaceutically effective amount. In some embodiments, the pharmaceutically effective amount of chlorpheniramine ranges from 0.01% w/v to 0.10% w/v. In some embodiments, the aqueous-based composition contains chlorpheniramine, or a pharmaceutically acceptable salt, ester, hydrate, or solvate thereof, in an amount that is less than about 0.1% w/v, less than about 0.099% w/v, less than about 0.098% w/v, less than about 0.097% w/v, less than about 0.096% w/v, less than about 0.095% w/v, less than about 0.094% w/v, less than about 0.093% w/v, less than about 0.092% w/v, less than about 0.091% w/v, less than about 0.09% w/v, less than about 0.089% w/v, less than about 0.088% w/v, less than about 0.087% w/v, less than about 0.086% w/v, less than about 0.085% w/v, less than about 0.084% w/v, less than about 0.083% w/v, less than about 0.082% w/v, less than about 0.081% w/v, less than about 0.08% w/v, less than about 0.079% w/v, less than about 0.078% w/v, less than about 0.077% w/v, less than about 0.076% w/v, less than about 0.075% w/v, less than about 0.074% w/v, less than about 0.073% w/v, less than about 0.072% w/v, less than about 0.071% w/v, less than about 0.07% w/v, less than about 0.069% w/v, less than about 0.068% w/v, less than about 0.067% w/v, less than about 0.066% w/v, less than about 0.065% w/v, less than about 0.064% w/v, less than about 0.063% w/v, less than about 0.062% w/v, less than about 0.061% w/v, less than about 0.06% w/v, less than about 0.059% w/v, less than about 0.058% w/v, less than about 0.057% w/v, less than about 0.056% w/v, less than about 0.055% w/v, less than about 0.054% w/v, less than about 0.053% w/v, less than about 0.052% w/v, less than about 0.051% w/v, 0.05% w/v, less than about 0.049% w/v, less than about 0.048% w/v, less than about 0.047% w/v, less than about 0.046% w/v, less than about 0.045% w/v, less than about 0.044% w/v, less than about 0.043% w/v, less than about 0.042% w/v, less than about 0.041% w/v, less than about 0.04% w/v, less than about 0.039% w/v, less than about 0.038% w/v, less than about 0.037% w/v, less than about 0.036% w/v, less than about 0.035% w/v, less than about 0.034% w/v, less than about 0.033% w/v, less than about 0.032% w/v, less than about 0.031% w/v, less than about 0.03% w/v, less than about 0.029% w/v, less than about 0.028% w/v, less than about 0.027% w/v, less than about 0.026% w/v, less than about 0.025% w/v, less than about 0.024% w/v, less than about 0.023% w/v, less than about 0.022% w/v, less than about 0.021% w/v, less than about 0.02% w/v, less than about 0.019% w/v, less than about 0.018% w/v, less than about 0.017% w/v, less than about 0.016% w/v, less than about 0.015% w/v, less than about 0.014% w/v, less than about 0.013% w/v, less than about 0.012% w/v, less than about 0.011% w/v, or less than about 0.01% w/v.

In some embodiments, the aqueous-based composition contains chlorpheniramine, or a pharmaceutically acceptable salt, ester, hydrate, or solvate thereof, in an amount that is greater than about 0.01% w/v, greater than about 0.011% w/v, greater than about 0.012% w/v, greater than about 0.013% w/v, greater than about 0.014% w/v, greater than about 0.015% w/v, greater than about 0.016% w/v, greater than about 0.017% w/v, greater than about 0.018% w/v, greater than about 0.019% w/v, greater than about 0.02% w/v, greater than about 0.021% w/v, greater than about 0.022% w/v, greater than about 0.023% w/v, greater than about 0.024% w/v, greater than about 0.025% w/v, greater than about 0.026% w/v, greater than about 0.027% w/v, greater than about 0.028% w/v, greater than about 0.029% w/v, greater than about 0.03% w/v, greater than about 0.031% w/v, greater than about 0.032% w/v, greater than about 0.033% w/v, greater than about 0.034% w/v, greater than about 0.035% w/v, greater than about 0.036% w/v, greater than about 0.037% w/v, greater than about 0.038% w/v, greater than about 0.039% w/v, greater than about 0.04% w/v, greater than about 0.041% w/v, greater than about 0.042% w/v, greater than about 0.043% w/v, greater than about 0.044% w/v, greater than about 0.045% w/v, greater than about 0.046% w/v, greater than about 0.047% w/v, greater than about 0.048% w/v, greater than about 0.049% w/v, greater than about 0.05% w/v, greater than about 0.051% w/v, greater than about 0.052% w/v, greater than about 0.053% w/v, greater than about 0.054% w/v, greater than about 0.055% w/v, greater than about 0.056% w/v, greater than about 0.057% w/v, greater than about 0.058% w/v, greater than about 0.059% w/v, greater than about 0.06% w/v, greater than about 0.061% w/v, greater than about 0.062% w/v, greater than about 0.063% w/v, greater than about 0.064% w/v, greater than about 0.065% w/v, greater than about 0.066% w/v, greater than about 0.067% w/v, greater than about 0.068% w/v, greater than about 0.069% w/v, greater than about 0.07% w/v, greater than about 0.071% w/v, greater than about 0.072% w/v, greater than about 0.073% w/v, greater than about 0.074% w/v, greater than about 0.075% w/v, greater than about 0.076% w/v, greater than about 0.077% w/v, greater than about 0.078% w/v, greater than about 0.079% w/v, greater than about 0.08% w/v, greater than about 0.081% w/v, greater than about 0.082% w/v, greater than about 0.083% w/v, greater than about 0.084% w/v, greater than about 0.085% w/v, greater than about 0.086% w/v, greater than about 0.087% w/v, greater than about 0.088% w/v, greater than about 0.089% w/v, greater than about 0.09% w/v, greater than about 0.091% w/v, greater than about 0.092% w/v, greater than about 0.093% w/v, greater than about 0.094% w/v, greater than about 0.095% w/v, greater than about 0.096% w/v, greater than about 0.097% w/v, greater than about 0.098% w/v, greater than about 0.099% w/v, or greater than about 0.1% w/v.

In some embodiments, the aqueous-based composition contains chlorpheniramine, or a pharmaceutically acceptable salt, ester, hydrate, or solvate thereof, in an amount that has a range of % w/v having an upper limit of about 0.1% w/v, 0.09% w/v, 0.08% w/v, 0.07% w/v, 0.06% w/v, 0.05% w/v, 0.04% w/v, 0.03% w/v, 0.02% w/v, or 0.01% w/v, and an independently selected lower limit of about 0.01% w/v, 0.02% w/v, 0.03% w/v, 0.04% w/v, 0.05% w/v, 0.06% w/v, 0.07% w/v, 0.08% w/v, 0.09% w/v, or 0.1% w/v, where the lower limit is less than the upper limit. In some embodiments, the aqueous-based composition contains chlorpheniramine, or a pharmaceutically acceptable salt, ester, hydrate, or solvate thereof, in an amount of about 0.01% w/v, about 0.02% w/v, about 0.03% w/v, about 0.04% w/v, about 0.05% w/v, about 0.06% w/v, about 0.07% w/v, about 0.08% w/v, about 0.09% w/v, or about 0.1% w/v.

In some embodiments, the aqueous-based composition contains chlorpheniramine, or a pharmaceutically acceptable salt, ester, hydrate, or solvate thereof (e.g., chlorpheniramine maleate, etc.), in a pharmaceutically effective amount. In some embodiments, the pharmaceutically effective amount of chlorpheniramine ranges from 0.2 mg/ml to 100 mg/ml. In some embodiments, the aqueous-based composition contains chlorpheniramine, or a pharmaceutically acceptable salt, ester, hydrate, or solvate thereof, in an amount that is less than about 100 mg/ml, less than about 95 mg/ml, less than about 90 mg/ml, less than about 85 mg/ml, less than about 80 mg/ml, less than about 75 mg/ml, less than about 70 mg/ml, less than about 65 mg/ml, less than about 60 mg/ml, less than about 55 mg/ml, less than about 50 mg/ml, less than about 45 mg/ml, less than about 40 mg/ml, less than about 35 mg/ml, less than about 30 mg/ml, less than about 25 mg/ml, less than about 20 mg/ml, less than about 15 mg/ml, less than about 10 mg/ml less than about 9 mg/ml, less than about 8 mg/ml, less than about 7 mg/ml, less than about 6 mg/ml, less than about 5 mg/ml, less than about 4 mg/ml, less than about 3 mg/ml, less than about 2 mg/ml, less than about 1 mg/ml, less than about 0.9 mg/ml, less than about 0.8 mg/ml, less than about 0.7 mg/ml, less than about 0.6 mg/ml, less than about 0.5 mg/ml, less than about 0.4 mg/ml, less than about 0.3 mg/ml, or less than about 0.2 mg/ml.

In some embodiments, the aqueous-based composition contains chlorpheniramine, or a pharmaceutically acceptable salt, ester, hydrate, or solvate thereof, in an amount that is greater than about 0.2 mg/ml, greater than about 0.3 mg/ml, greater than about 0.4 mg/ml, greater than about 0.5 mg/ml, greater than about 0.6 mg/ml, greater than about 0.7 mg/ml, greater than about 0.8 mg/ml, greater than about 0.9 mg/ml, greater than about 1 mg/ml, greater than about 2 mg/ml, greater than about 3 mg/ml, greater than about 4 mg/ml, greater than about 5 mg/ml, greater than about 6 mg/ml, greater than about 7 mg/ml, greater than about 8 mg/ml, greater than about 9 mg/ml, greater than about 10 mg/ml, greater than about 15 mg/ml, greater than about 20 mg/ml, greater than about 25 mg/ml, greater than about 30 mg/ml, greater than about 35 mg/ml, greater than about 40 mg/ml, greater than about 45 mg/ml, greater than about 50 mg/ml, greater than about 55 mg/ml, greater than about 60 mg/ml, greater than about 65 mg/ml, greater than about 70 mg/ml, greater than about 75 mg/ml, greater than about 80 mg/ml, greater than about 85 mg/ml, greater than about 90 mg/ml, greater than about 95 mg/ml, or greater than about 100 mg/ml.

In some embodiments, the aqueous-based composition contains chlorpheniramine, or a pharmaceutically acceptable salt, ester, hydrate, or solvate thereof, in an amount that has a range of mg/ml having an upper limit of about 100 mg/ml, 80 mg/ml, 60 mg/ml, 40 mg/ml, 20 mg/ml, 10 mg/ml, 1 mg/ml, 0.8 mg/ml, 0.4 mg/ml, or 0.2 mg/ml and an independently selected lower limit of about 0.2 mg/ml, 0.4 mg/ml, 0.8 mg/ml, 1 mg/ml, 10 mg/ml, 20 mg/ml, 30 mg/ml, 40 mg/ml, 50 mg/ml, 60 mg/ml, 70 mg/ml, 80 mg/ml, 90 mg/ml, or 100 mg/ml, where the lower limit is less than the upper limit. In some embodiments, the aqueous-based composition contains chlorpheniramine, or a pharmaceutically acceptable salt, ester, hydrate, or solvate thereof, in an amount of about 0.2 mg/ml, about 0.4 mg/ml, about 0.8 mg/ml, about 1 mg/ml, about 10 mg/ml, about 20 mg/ml, about 30 mg/ml, about 40 mg/ml, about 50 mg/ml, about 60 mg/ml, about 70 mg/ml, about 80 mg/ml, about 90 mg/ml, or about 100 mg/ml. In some embodiments, the aqueous-based composition contains chlorpheniramine, or a pharmaceutically acceptable salt, ester, hydrate, or solvate thereof, in an amount of about 0.4 mg/ml.

In some embodiments, the aqueous-based composition contains brompheniramine, or a pharmaceutically acceptable salt, ester, hydrate, or solvate thereof (e.g., brompheniramine maleate, etc.), in a pharmaceutically effective amount. In some embodiments, the pharmaceutically effective amount of brompheniramine ranges from 0.1 mg/ml to 100 mg/ml. In some embodiments, the aqueous-based composition contains brompheniramine, or a pharmaceutically acceptable salt, ester, hydrate, or solvate thereof, in an amount that is less than about 100 mg/ml, less than about 95 mg/ml, less than about 90 mg/ml, less than about 85 mg/ml, less than about 80 mg/ml, less than about 75 mg/ml, less than about 70 mg/ml, less than about 65 mg/ml, less than about 60 mg/ml, less than about 55 mg/ml, less than about 50 mg/ml, less than about 45 mg/ml, less than about 40 mg/ml, less than about 35 mg/ml, less than about 30 mg/ml, less than about 25 mg/ml, less than about 20 mg/ml, less than about 15 mg/ml, less than about 10 mg/ml, less than about 9 mg/ml, less than about 8 mg/ml, less than about 7 mg/ml, less than about 6 mg/ml, less than about 5 mg/ml, less than about 4.8 mg/ml, less than about 4.6 mg/ml, less than about 4.4 mg/ml, less than about 4.2 mg/ml, less than about 4 mg/ml, less than about 3.8 mg/ml, less than about 3.6 mg/ml, less than about 3.4 mg/ml, less than about 3.2 mg/ml, less than about 3 mg/ml, less than about 2.8 mg/ml, less than about 2.6 mg/ml, less than about 2.4 mg/ml, less than about 2.2 mg/ml, less than about 2 mg/ml, less than about 1.8 mg/ml, less than about 1.6 mg/ml, less than about 1.4 mg/ml, less than about 1.2 mg/ml, less than about 1 mg/ml, less than about 0.9 mg/ml, less than about 0.8 mg/ml, less than about 0.7 mg/ml, less than about 0.6 mg/ml, less than about 0.5 mg/ml, less than about 0.4 mg/ml, less than about 0.3 mg/ml, less than about 0.2 mg/ml, or less than about 0.1 mg/ml.

In some embodiments, the aqueous-based composition contains brompheniramine, or a pharmaceutically acceptable salt, ester, hydrate, or solvate thereof, in an amount that is greater than about 0.1 mg/ml, greater than about 0.2 mg/ml, greater than about 0.3 mg/ml, greater than about 0.4 mg/ml, greater than about 0.5 mg/ml, greater than about 0.6 mg/ml, greater than about 0.7 mg/ml, greater than about 0.8 mg/ml, greater than about 0.9 mg/ml, greater than about 1 mg/ml, greater than about 1.2 mg/ml, greater than about 1.4 mg/ml, greater than about 1.6 mg/ml, greater than about 1.8 mg/ml, greater than about 2 mg/ml, greater than about 2.2 mg/ml, greater than about 2.4 mg/ml, greater than about 2.6 mg/ml, greater than about 2.8 mg/ml, greater than about 3 mg/ml, greater than about 3.2 mg/ml, greater than about 3.4 mg/ml, greater than about 3.6 mg/ml, greater than about 3.8 mg/ml, greater than about 4 mg/ml, greater than about 4.2 mg/ml, greater than about 4.4 mg/ml, greater than about 4.6 mg/ml, greater than about 4.8 mg/ml, greater than about 5 mg/ml, greater than about 6 mg/ml, greater than about 7 mg/ml, greater than about 8 mg/ml, greater than about 9 mg/ml, greater than about 10 mg/ml, greater than about 15 mg/ml, greater than about 20 mg/ml, greater than about 25 mg/ml, greater than about 30 mg/ml, greater than about 35 mg/ml, greater than about 40 mg/ml, greater than about 45 mg/ml, greater than about 50 mg/ml, greater than about 55 mg/ml, greater than about 60 mg/ml, greater than about 65 mg/ml, greater than about 70 mg/ml, greater than about 75 mg/ml, greater than about 80 mg/ml, greater than about 85 mg/ml, greater than about 90 mg/ml, greater than about 95 mg/ml, or greater than about 100 mg/ml.

In some embodiments, the aqueous-based composition contains brompheniramine, or a pharmaceutically acceptable salt, ester, hydrate, or solvate thereof, in an amount that has a range of mg/ml having an upper limit of about 100 mg/ml, 80 mg/ml, 60 mg/ml, 40 mg/ml, 20 mg/ml, 10 mg/ml, 2.4 mg/ml, 1.2 mg/ml, 1 mg/ml, 0.8 mg/ml, 0.6 mg/ml, 0.4 mg/ml, 0.2 mg/ml, or 0.1 mg/ml and an independently selected lower limit of about 0.1 mg/ml, 0.2 mg/ml, 0.4 mg/ml, 0.6 mg/ml, 0.8 mg/ml, 1 mg/ml, 1.2 mg/ml, 2.4 mg/ml, 10 mg/ml, 20 mg/ml, 30 mg/ml, 40 mg/ml, 50 mg/ml, 60 mg/ml, 70 mg/ml, 80 mg/ml, 90 mg/ml, or 100 mg/ml, where the lower limit is less than the upper limit. In some embodiments, the aqueous-based composition contains brompheniramine, or a pharmaceutically acceptable salt, ester, hydrate, or solvate thereof, in an amount of about 0.1 mg/ml, about 0.2 mg/ml, about 0.4 mg/ml, about 0.6 mg/ml, about 0.8 mg/ml, about 1 mg/ml, about 1.2 mg/ml, about 2.4 mg/ml, about 10 mg/ml, about 20 mg/ml, about 30 mg/ml, about 40 mg/ml, about 50 mg/ml, about 60 mg/ml, about 70 mg/ml, about 80 mg/ml, about 90 mg/ml, or about 100 mg/ml. In some embodiments, the aqueous-based composition contains brompheniramine, or a pharmaceutically acceptable salt, ester, hydrate, or solvate thereof, in an amount of about 0.2 mg/ml. In some embodiments, the aqueous-based composition contains brompheniramine, or a pharmaceutically acceptable salt, ester, hydrate, or solvate thereof, in an amount of about 0.4 mg/ml. In some embodiments, the aqueous-based composition contains brompheniramine, or a pharmaceutically acceptable salt, ester, hydrate, or solvate thereof, in an amount of about 0.8 mg/ml. In some embodiments, the aqueous-based composition contains brompheniramine, or a pharmaceutically acceptable salt, ester, hydrate, or solvate thereof, in an amount of about 1.2 mg/ml. In some embodiments, the aqueous-based composition contains brompheniramine, or a pharmaceutically acceptable salt, ester, hydrate, or solvate thereof, in an amount of about 2.4 mg/ml.

In some embodiments, the aqueous-based composition contains loratadine, or a pharmaceutically acceptable salt, ester, hydrate, or solvate thereof in a pharmaceutically effective amount. In some embodiments, the pharmaceutically effective amount of loratadine ranges from 0.4 mg/ml to 1.2 mg/ml. In some embodiments, the aqueous-based composition contains loratadine, or a pharmaceutically acceptable salt, ester, hydrate, or solvate thereof, in an amount that is less than about 1.2 mg/ml, less than about 1.18 mg/ml, less than about 1.16 mg/ml, less than about 1.14 mg/ml, less than about 1.12 mg/ml, less than about 1.1 mg/ml, less than about 1.08 mg/ml, less than about 1.06 mg/ml, less than about 1.04 mg/ml, less than about 1.02 mg/ml, less than less than about 1 mg/ml, less than about 0.99 mg/ml, less than about 0.98 mg/ml, less than about 0.97 mg/ml, less than about 0.96 mg/ml, less than about 0.95 mg/ml, less than about 0.94 mg/ml, less than about 0.93 mg/ml, less than about 0.92 mg/ml, less than about 0.91 mg/ml, less than about 0.9 mg/ml, less than about 0.89 mg/ml, less than about 0.88 mg/ml, less than about 0.87 mg/ml, less than about 0.86 mg/ml, less than about 0.85 mg/ml, less than about 0.84 mg/ml, less than about 0.83 mg/ml, less than about 0.82 mg/ml, less than about 0.81 mg/ml, less than about 0.8 mg/ml, less than about 0.79 mg/ml, less than about 0.78 mg/ml, less than about 0.77 mg/ml, less than about 0.76 mg/ml, less than about 0.75 mg/ml, less than about 0.74 mg/ml, less than about 0.73 mg/ml, less than about 0.72 mg/ml, less than about 0.71 mg/ml, less than about 0.7 mg/ml, less than about 0.69 mg/ml, less than about 0.68 mg/ml, less than about 0.67 mg/ml, less than about 0.66 mg/ml, less than about 0.65 mg/ml, less than about 0.64 mg/ml, less than about 0.63 mg/ml, less than about 0.62 mg/ml, less than about 0.61 mg/ml, less than about 0.6 mg/ml, less than about 0.59 mg/ml, less than about 0.58 mg/ml, less than about 0.57 mg/ml, less than about 0.56 mg/ml, less than about 0.55 mg/ml, less than about 0.54 mg/ml, less than about 0.53 mg/ml, less than about 0.52 mg/ml, less than about 0.51 mg/ml, less than about 0.5 mg/ml, less than about 0.49 mg/ml, less than about 0.48 mg/ml, less than about 0.47 mg/ml, less than about 0.46 mg/ml, less than about 0.45 mg/ml, less than about 0.44 mg/ml, less than about 0.43 mg/ml, less than about 0.42 mg/ml, less than about 0.41 mg/ml, or less than about 0.4 mg/ml.

In some embodiments, the aqueous-based composition contains loratadine, or a pharmaceutically acceptable salt, ester, hydrate, or solvate thereof, in an amount that is greater than about 0.4 mg/ml, greater than about 0.41 mg/ml, greater than about 0.42 mg/ml, greater than about 0.43 mg/ml, greater than about 0.44 mg/ml, greater than about 0.45 mg/ml, greater than about 0.46 mg/ml, greater than about 0.47 mg/ml, greater than about 0.48 mg/ml, greater than about 0.49 mg/ml, greater than about 0.5 mg/ml, greater than about 0.51 mg/ml, greater than about 0.52 mg/ml, greater than about 0.53 mg/ml, greater than about 0.54 mg/ml, greater than about 0.55 mg/ml, greater than about 0.56 mg/ml, greater than about 0.57 mg/ml, greater than about 0.58 mg/ml, greater than about 0.59 mg/ml, greater than about 0.6 mg/ml, greater than about 0.61 mg/ml, greater than about 0.62 mg/ml, greater than about 0.63 mg/ml, greater than about 0.64 mg/ml, greater than about 0.65 mg/ml, greater than about 0.66 mg/ml, greater than about 0.67 mg/ml, greater than about 0.68 mg/ml, greater than about 0.69 mg/ml, greater than about 0.7 mg/ml, greater than about 0.71 mg/ml, greater than about 0.72 mg/ml, greater than about 0.73 mg/ml, greater than about 0.74 mg/ml, greater than about 0.75 mg/ml, greater than about 0.76 mg/ml, greater than about 0.77 mg/ml w/v, greater than about 0.78 mg/ml, greater than about 0.79 mg/ml, greater than about 0.8 mg/ml, greater than about 0.81 mg/ml, greater than about 0.82 mg/ml, greater than about 0.83 mg/ml, greater than about 0.84 mg/ml, greater than about 0.85 mg/ml, greater than about 0.86 mg/ml, greater than about 0.87 mg/ml, greater than about 0.88 mg/ml, greater than about 0.89 mg/ml, greater than about 0.9 mg/ml, greater than about 0.91 mg/ml, greater than about 0.92 mg/ml, greater than about 0.93 mg/ml, greater than about 0.94 mg/ml, greater than about 0.95 mg/ml, greater than about 0.96 mg/ml, greater than about 0.97 mg/ml, greater than about 0.98 mg/ml, greater than about 0.99 mg/ml, greater than about 1 mg/ml, greater than about 1.02 mg/ml, greater than about 1.04 mg/ml, greater than about 1.06 mg/ml, greater than about 1.08 mg/ml, greater than about 1.1 mg/ml, greater than about 1.12 mg/ml, greater than about 1.14 mg/ml, greater than about 1.16 mg/ml, greater than about 1.18 mg/ml, or greater than about 1.2 mg/ml.

In some embodiments, the aqueous-based composition contains loratadine, or a pharmaceutically acceptable salt, ester, hydrate, or solvate thereof, in an amount that has a range of mg/ml having an upper limit of about 1.2 mg/ml, 1.1 mg/ml, 1 mg/ml, 0.9 mg/ml, 0.8 mg/ml, 0.7 mg/ml, 0.6 mg/ml, 0.5 mg/ml, or 0.4 mg/ml and an independently selected lower limit of about 0.4 mg/ml, 0.5 mg/ml, 0.6 mg/ml, 0.7 mg/ml, 0.8 mg/ml, 0.9 mg/ml, 1 mg/ml, 1.1 mg/ml, or 1.2 mg/ml where the lower limit is less than the upper limit. In some embodiments, the aqueous-based composition contains loratadine, or a pharmaceutically acceptable salt, ester, hydrate, or solvate thereof, in an amount of about 0.4 mg/ml, about 0.5 mg/ml, about 0.6 mg/ml, about 0.7 mg/ml, about 0.8 mg/ml, about 0.9 mg/ml, about 1 mg/ml, about 1.1 mg/ml, or about 1.2 mg/ml. In some embodiments, the aqueous-based composition contains loratadine, or a pharmaceutically acceptable salt, ester, hydrate, or solvate thereof, in an amount of about 1 mg/ml.

In some embodiments, the aqueous-based composition contains naproxen, or a pharmaceutically acceptable salt, ester, hydrate, or solvate thereof (e.g., naproxen sodium, etc.), in a pharmaceutically effective amount. In some embodiments, the pharmaceutically effective amount of naproxen ranges from about 0.2 mg/ml to about 100 mg/ml. In some embodiments, the aqueous-based composition contains naproxen, or a pharmaceutically acceptable salt, ester, hydrate, or solvate thereof, in an amount that is less than about 100 mg/ml, less than about 95 mg/ml, less than about 90 mg/ml, less than about 85 mg/ml, less than about 80 mg/ml, less than about 75 mg/ml, less than about 70 mg/ml, less than about 65 mg/ml, less than about 60 mg/ml, less than about 55 mg/ml, less than about 50 mg/ml, less than about 45 mg/ml, less than about 40 mg/ml, less than about 35 mg/ml, less than about 30 mg/ml, less than about 25 mg/ml, less than about 20 mg/ml, less than about 15 mg/ml, less than about 10 mg/ml less than about 9 mg/ml, less than about 8 mg/ml, less than about 7 mg/ml, less than about 6 mg/ml, less than about 5 mg/ml, less than about 4 mg/ml, less than about 3 mg/ml, less than about 2 mg/ml, less than about 1 mg/ml, less than about 0.9 mg/ml, less than about 0.8 mg/ml, less than about 0.7 mg/ml, less than about 0.6 mg/ml, less than about 0.5 mg/ml, less than about 0.4 mg/ml, less than about 0.3 mg/ml, or less than about 0.2 mg/ml.

In some embodiments, the aqueous-based composition contains naproxen, or a pharmaceutically acceptable salt, ester, hydrate, or solvate thereof, in an amount that is greater than about 0.2 mg/ml, greater than about 0.3 mg/ml, greater than about 0.4 mg/ml, greater than about 0.5 mg/ml, greater than about 0.6 mg/ml, greater than about 0.7 mg/ml, greater than about 0.8 mg/ml, greater than about 0.9 mg/ml, greater than about 1 mg/ml, greater than about 2 mg/ml, greater than about 3 mg/ml, greater than about 4 mg/ml, greater than about 5 mg/ml, greater than about 6 mg/ml, greater than about 7 mg/ml, greater than about 8 mg/ml, greater than about 9 mg/ml, greater than about 10 mg/ml, greater than about 15 mg/ml, greater than about 20 mg/ml, greater than about 25 mg/ml, greater than about 30 mg/ml, greater than about 35 mg/ml, greater than about 40 mg/ml, greater than about 45 mg/ml, greater than about 50 mg/ml, greater than about 55 mg/ml, greater than about 60 mg/ml, greater than about 65 mg/ml, greater than about 70 mg/ml, greater than about 75 mg/ml, greater than about 80 mg/ml, greater than about 85 mg/ml, greater than about 90 mg/ml, greater than about 95 mg/ml, or greater than about 100 mg/ml.

In some embodiments, the aqueous-based composition contains naproxen in an amount that has a range of mg/ml having an upper limit of about 100 mg/ml, 80 mg/ml, 60 mg/ml, 40 mg/ml, 25 mg/ml, 20 mg/ml, 10 mg/ml, 1 mg/ml, 0.8 mg/ml, 0.4 mg/ml, or 0.2 mg/ml and an independently selected lower limit of about 0.2 mg/ml, 0.4 mg/ml, 0.8 mg/ml, 1 mg/ml, 10 mg/ml, 20 mg/ml, 25 mg/ml, 30 mg/ml, 40 mg/ml, 50 mg/ml, 60 mg/ml, 70 mg/ml, 80 mg/ml, 90 mg/ml, or 100 mg/ml, where the lower limit is less than the upper limit. In some embodiments, the aqueous-based composition contains naproxen in an amount of about 0.2 mg/ml, about 0.4 mg/ml, about 0.8 mg/ml, about 1 mg/ml, about 10 mg/ml, about 20 mg/ml, about 25 mg/ml, about 30 mg/ml, about 40 mg/ml, about 50 mg/ml, about 60 mg/ml, about 70 mg/ml, about 80 mg/ml, about 90 mg/ml, or about 100 mg/ml. In some embodiments, the aqueous-based composition contains naproxen in an amount of about 25 mg/ml.

In some embodiments, the aqueous-based composition contains ibuprofen, or a pharmaceutically acceptable salt, ester, hydrate, or solvate thereof (e.g., ibuprofen lysinate, etc.), in a pharmaceutically effective amount. In some embodiments, the pharmaceutically effective amount of ibuprofen ranges from 15 mg/ml to 100 mg/ml. In some embodiments, the aqueous-based composition contains ibuprofen, or a pharmaceutically acceptable salt, ester, hydrate, or solvate thereof, in an amount that is less than about 100 mg/ml, less than about 65 mg/ml, less than about 90 mg/ml, less than about 85 mg/ml, less than about 80 mg/ml, less than about 75 mg/ml, less than about 70 mg/ml, less than about 65 mg/ml, less than about 60 mg/ml, less than about 55 mg/ml, less than about 50 mg/ml, less than about 45 mg/ml, less than about 40 mg/ml, less than about 39 mg/ml, less than about 38 mg/ml, less than about 37 mg/ml, less than about 36 mg/ml, less than about 35 mg/ml, less than about 34 mg/ml, less than about 33 mg/ml, less than about 32 mg/ml, less than about 31 mg/ml, less than about 30 mg/ml, less than about 29 mg/ml, less than about 28 mg/ml, less than about 27 mg/ml, less than about 26 mg/ml, less than about 25 mg/ml, less than about 24 mg/ml, less than about 23 mg/ml, less than about 22 mg/ml less than about 21 mg/ml, less than about 20 mg/ml, less than about 19 mg/ml, less than about 18 mg/ml, less than about 17 mg/ml, less than about 16 mg/ml, or less than about 15 mg/ml.

In some embodiments, the aqueous-based composition contains ibuprofen, or a pharmaceutically acceptable salt, ester, hydrate, or solvate thereof, in an amount that is greater than about 15 mg/ml, greater than about 16 mg/ml, greater than about 17 mg/ml, greater than about 18 mg/ml, greater than about 19 mg/ml, greater than about 20 mg/ml, greater than about 21 mg/ml, greater than about 22 mg/ml, greater than about 23 mg/ml, greater than about 24 mg/ml, greater than about 25 mg/ml, greater than about 26 mg/ml, greater than about 27 mg/ml, greater than about 28 mg/ml, greater than about 29 mg/ml, greater than about 30 mg/ml, greater than about 31 mg/ml, greater than about 32 mg/ml, greater than about 33 mg/ml, greater than about 34 mg/ml, greater than about 35 mg/ml, greater than about 36 mg/ml, greater than about 37 mg/ml, greater than about 38 mg/ml, greater than about 39 mg/ml, greater than about 40 mg/ml, greater than about 45 mg/ml, greater than about 50 mg/ml, greater than about 55 mg/ml, greater than about 60 mg/ml, greater than about 65 mg/ml, greater than about 70 mg/ml, greater than about 75 mg/ml, greater than about 80 mg/ml, greater than about 85 mg/ml, greater than about 90 mg/ml, greater than about 95 mg/ml, or greater than about 100 mg/ml.

In some embodiments, the aqueous-based composition contains ibuprofen, or a pharmaceutically acceptable salt, ester, hydrate, or solvate thereof, in an amount that has a range of mg/ml having an upper limit of about 100 mg/ml, 90 mg/ml, 80 mg/ml, 70 mg/ml, 60 mg/ml, 50 mg/ml, 40 mg/ml, 30 mg/ml, 20 mg/ml, or 15 mg/ml and an independently selected lower limit of about 15 mg/ml, 20 mg/ml, 30 mg/ml, 40 mg/ml, 50 mg/ml, 60 mg/ml, 70 mg/ml, 80 mg/ml, 90 mg/ml, or 100 mg/ml, where the lower limit is less than the upper limit. In some embodiments, the aqueous-based composition contains ibuprofen, or a pharmaceutically acceptable salt, ester, hydrate, or solvate thereof, in an amount of about 15 mg/ml, about 20 mg/ml, about 30 mg/ml, about 40 mg/ml, about 50 mg/ml, about 60 mg/ml, about 70 mg/ml, about 80 mg/ml, about 90 mg/ml, or about 100 mg/ml. In some embodiments, the aqueous-based composition contains ibuprofen, or a pharmaceutically acceptable salt, ester, hydrate, or solvate thereof, in an amount of about 20 mg/ml.

In some embodiments, the aqueous-based composition contains acetaminophen (paracetamol), or a pharmaceutically acceptable salt, ester, hydrate, or solvate thereof in a pharmaceutically effective amount. In some embodiments, the pharmaceutically effective amount of acetaminophen ranges from 10 mg/ml to 110 mg/ml. In some embodiments, the aqueous-based composition contains acetaminophen, or a pharmaceutically acceptable salt, ester, hydrate, or solvate thereof, in an amount that is less than about 110 mg/ml, less than about 105 mg/ml, less than about 100 mg/ml, less than about 95 mg/ml, less than about 90 mg/ml, less than about 85 mg/ml, less than about 80 mg/ml, less than about 75 mg/ml, less than about 70 mg/ml, less than about 65 mg/ml, less than about 60 mg/ml, less than about 55 mg/ml, less than about 50 mg/ml, less than about 45 mg/ml, less than about 40 mg/ml, less than about 39 mg/ml, less than about 38 mg/ml, less than about 37 mg/ml, less than about 36 mg/ml, less than about 35 mg/ml, less than about 34 mg/ml, less than about 33 mg/ml, less than about 32 mg/ml, less than about 31 mg/ml, less than about 30 mg/ml, less than about 29 mg/ml, less than about 28 mg/ml, less than about 27 mg/ml, less than about 26 mg/ml, less than about 25 mg/ml, less than about 24 mg/ml, less than about 23 mg/ml, less than about 22 mg/ml, less than about 21 mg/ml, less than about 20 mg/ml, less than about 19 mg/ml, less than about 18 mg/ml, less than about 17 mg/ml, less than about 16 mg/ml, less than about 15 mg/ml, less than about 14 mg/ml, less than about 13 mg/ml, less than about 12 mg/ml, less than about 11 mg/ml, or less than about 10 mg/ml.

In some embodiments, the aqueous-based composition contains acetaminophen, or a pharmaceutically acceptable salt, ester, hydrate, or solvate thereof, in an amount that is greater than about 10 mg/ml, greater than about 11 mg/ml, greater than about 12 mg/ml, greater than about 13 mg/ml, greater than about 14 mg/ml, greater than about 15 mg/ml, greater than about 16 mg/ml, greater than about 17 mg/ml, greater than about 18 mg/ml, greater than about 19 mg/ml, greater than about 20 mg/ml, greater than about 21 mg/ml, greater than about 22 mg/ml, greater than about 23 mg/ml, greater than about 24 mg/ml, greater than about 25 mg/ml, greater than about 26 mg/ml, greater than about 27 mg/ml, greater than about 28 mg/ml, greater than about 29 mg/ml, greater than about 30 mg/ml, greater than about 31 mg/ml, greater than about 32 mg/ml, greater than about 33 mg/ml, greater than about 34 mg/ml, greater than about 35 mg/ml, greater than about 36 mg/ml, greater than about 37 mg/ml, greater than about 38 mg/ml, greater than about 39 mg/ml, greater than about 40 mg/ml, greater than about 45 mg/ml, greater than about 50 mg/ml, greater than about 55 mg/ml, greater than about 60 mg/ml, greater than about 65 mg/ml, greater than about 70 mg/ml, greater than about 75 mg/ml, greater than about 80 mg/ml, greater than about 85 mg/ml, greater than about 90 mg/ml, greater than about 95 mg/ml, greater than about 100 mg/ml, greater than about 105 mg/ml, or greater than about 110 mg/ml.

In some embodiments, the aqueous-based composition contains acetaminophen, or a pharmaceutically acceptable salt, ester, hydrate, or solvate thereof, in an amount that has a range of mg/ml having an upper limit of about 110 mg/ml, 105 mg/ml, 100 mg/ml, 95 mg/ml, 90 mg/ml, 85 mg/ml, 80 mg/ml, 75 mg/ml, 70 mg/ml, 65 mg/ml, 60 mg/ml, 55 mg/ml, 50 mg/ml, 45 mg/ml, 40 mg/ml, 35 mg/ml, 33 mg/ml, 32 mg/ml, 30 mg/ml, 28 mg/ml, 26 mg/ml, 24 mg/ml, 22 mg/ml, 20 mg/ml, or 10 mg/ml, and an independently selected lower limit of about 10 mg/ml, 20 mg/ml, 22 mg/ml, 24 mg/ml, 26 mg/ml, 28 mg/ml, 30 mg/ml, 32 mg/ml, 33 mg/ml, 40 mg/ml, 45 mg/ml, 50 mg/ml, 55 mg/ml, 60 mg/ml, 65 mg/ml, 70 mg/ml, 75 mg/ml, 80 mg/ml, 85 mg/ml, 90 mg/ml, 95 mg/ml, 100 mg/ml, 105 mg/ml, or 110 mg/ml, where the lower limit is less than the upper limit. In some embodiments, the aqueous-based composition contains diphenhydramine, or a pharmaceutically acceptable salt, ester, hydrate, or solvate thereof, in an amount of about 10 mg/ml, about 20 mg/ml, about 22 mg/ml, about 24 mg/ml, about 26 mg/ml, about 28 mg/ml, about 30 mg/ml, about 32 mg/ml, about 33 mg/ml, about 40 mg/ml, about 45 mg/ml, about 50 mg/ml, about 55 mg/ml, about 60 mg/ml, about 65 mg/ml, about 70 mg/ml, about 75 mg/ml, about 80 mg/ml, about 85 mg/ml, about 90 mg/ml, about 95 mg/ml, about 100 mg/ml, about 105 mg/ml, or about 110 mg/ml. In some embodiments, the aqueous-based composition contains diphenhydramine, or a pharmaceutically acceptable salt, ester, hydrate, or solvate thereof, in an amount of about 32 mg/ml. In some embodiments, the aqueous-based composition contains diphenhydramine, or a pharmaceutically acceptable salt, ester, hydrate, or solvate thereof, in an amount of about 100 mg/ml.

In some embodiments, the aqueous-based composition contains guaifenesin, or a pharmaceutically acceptable salt, ester, hydrate, or solvate thereof, in a pharmaceutically effective amount. In some embodiments, the pharmaceutically effective amount of guaifenesin ranges from about 0.2 mg/ml to about 100 mg/ml. In some embodiments, the aqueous-based composition contains guaifenesin, or a pharmaceutically acceptable salt, ester, hydrate, or solvate thereof, in an amount that is less than about 100 mg/ml, less than about 95 mg/ml, less than about 90 mg/ml, less than about 85 mg/ml, less than about 80 mg/ml, less than about 75 mg/ml, less than about 70 mg/ml, less than about 65 mg/ml, less than about 60 mg/ml, less than about 55 mg/ml, less than about 50 mg/ml, less than about 45 mg/ml, less than about 40 mg/ml, less than about 35 mg/ml, less than about 30 mg/ml, less than about 25 mg/ml, less than about 20 mg/ml, less than about 15 mg/ml, less than about 10 mg/ml less than about 9 mg/ml, less than about 8 mg/ml, less than about 7 mg/ml, less than about 6 mg/ml, less than about 5 mg/ml, less than about 4 mg/ml, less than about 3 mg/ml, less than about 2 mg/ml, less than about 1 mg/ml, less than about 0.9 mg/ml, less than about 0.8 mg/ml, less than about 0.7 mg/ml, less than about 0.6 mg/ml, less than about 0.5 mg/ml, less than about 0.4 mg/ml, less than about 0.3 mg/ml, or less than about 0.2 mg/ml.

In some embodiments, the aqueous-based composition contains guaifenesin, or a pharmaceutically acceptable salt, ester, hydrate, or solvate thereof, in an amount that is greater than about 0.2 mg/ml, greater than about 0.3 mg/ml, greater than about 0.4 mg/ml, greater than about 0.5 mg/ml, greater than about 0.6 mg/ml, greater than about 0.7 mg/ml, greater than about 0.8 mg/ml, greater than about 0.9 mg/ml, greater than about 1 mg/ml, greater than about 2 mg/ml, greater than about 3 mg/ml, greater than about 4 mg/ml, greater than about 5 mg/ml, greater than about 6 mg/ml, greater than about 7 mg/ml, greater than about 8 mg/ml, greater than about 9 mg/ml, greater than about 10 mg/ml, greater than about 15 mg/ml, greater than about 20 mg/ml, greater than about 25 mg/ml, greater than about 30 mg/ml, greater than about 35 mg/ml, greater than about 40 mg/ml, greater than about 45 mg/ml, greater than about 50 mg/ml, greater than about 55 mg/ml, greater than about 60 mg/ml, greater than about 65 mg/ml, greater than about 70 mg/ml, greater than about 75 mg/ml, greater than about 80 mg/ml, greater than about 85 mg/ml, greater than about 90 mg/ml, greater than about 95 mg/ml, or greater than about 100 mg/ml.

In some embodiments, the aqueous-based composition contains guaifenesin, or a pharmaceutically acceptable salt, ester, hydrate, or solvate thereof, in an amount that has a range of mg/ml having an upper limit of about 100 mg/ml, 80 mg/ml, 60 mg/ml, 40 mg/ml, 20 mg/ml, 10 mg/ml, 1 mg/ml, 0.8 mg/ml, 0.4 mg/ml, or 0.2 mg/ml and an independently selected lower limit of about 0.2 mg/ml, 0.4 mg/ml, 0.8 mg/ml, 1 mg/ml, 10 mg/ml, 20 mg/ml, 30 mg/ml, 40 mg/ml, 50 mg/ml, 60 mg/ml, 70 mg/ml, 80 mg/ml, 90 mg/ml, or 100 mg/ml, where the lower limit is less than the upper limit. In some embodiments, the aqueous-based composition contains guaifenesin, or a pharmaceutically acceptable salt, ester, hydrate, or solvate thereof, in an amount of about 0.2 mg/ml, about 0.4 mg/ml, about 0.8 mg/ml, about 1 mg/ml, about 10 mg/ml, about 20 mg/ml, about 30 mg/ml, about 40 mg/ml, about 50 mg/ml, about 60 mg/ml, about 70 mg/ml, about 80 mg/ml, about 90 mg/ml, or about 100 mg/ml. In some embodiments, the aqueous-based composition contains guaifenesin, or a pharmaceutically acceptable salt, ester, hydrate, or solvate thereof, in an amount of about 20 mg/ml.

In some embodiments, the aqueous-based compositions may contain two APIs. In some embodiments, the two APIs are phenylephrine, or a pharmaceutically acceptable salt, ester, hydrate, or solvate thereof (e.g., phenylephrine HCl, phenylephrine bitartrate, etc.), and dextromethorphan, or a pharmaceutically acceptable salt, ester, hydrate, or solvate thereof (e.g., dextromethorphan HBr, etc.). In some embodiments, the aqueous-based composition contains phenylephrine, or a pharmaceutically acceptable salt, ester, hydrate, or solvate thereof in an amount that ranges from 0.01% w/v to about 0.05% w/v and dextromethorphan, or a pharmaceutically acceptable salt, ester, hydrate, or solvate thereof in an amount that ranges from 0.01% w/v to about 0.10% w/v. In some embodiments, the aqueous-based composition contains phenylephrine, or a pharmaceutically acceptable salt, ester, hydrate, or solvate thereof, in an amount that is less than about 0.05% w/v, less than about 0.049% w/v, less than about 0.048% w/v, less than about 0.047% w/v, less than about 0.046% w/v, less than about 0.045% w/v, less than about 0.044% w/v, less than about 0.043% w/v, less than about 0.042% w/v, less than about 0.041% w/v, less than about 0.04% w/v, less than about 0.039% w/v, less than about 0.038% w/v, less than about 0.037% w/v, less than about 0.036% w/v, less than about 0.035% w/v, less than about 0.034% w/v, less than about 0.033% w/v, less than about 0.032% w/v, less than about 0.031% w/v, less than about 0.03% w/v, less than about 0.029% w/v, less than about 0.028% w/v, less than about 0.027% w/v, less than about 0.026% w/v, less than about 0.025% w/v, less than about 0.024% w/v, less than about 0.023% w/v, less than about 0.022% w/v, less than about 0.021% w/v, less than about 0.02% w/v, less than about 0.019% w/v, less than about 0.018% w/v, less than about 0.017% w/v, less than about 0.016% w/v, less than about 0.015% w/v, less than about 0.014% w/v, less than about 0.013% w/v, less than about 0.012% w/v, less than about 0.011% w/v, or less than about 0.01% w/v, and dextromethorphan, or a pharmaceutically acceptable salt, ester, hydrate, or solvate thereof, in an amount that is less than about 0.1% w/v, less than about 0.099% w/v, less than about 0.098% w/v, less than about 0.097% w/v, less than about 0.096% w/v, less than about 0.095% w/v, less than about 0.094% w/v, less than about 0.093% w/v, less than about 0.092% w/v, less than about 0.091% w/v, less than about 0.09% w/v, less than about 0.089% w/v, less than about 0.088% w/v, less than about 0.087% w/v, less than about 0.086% w/v, less than about 0.085% w/v, less than about 0.084% w/v, less than about 0.083% w/v, less than about 0.082% w/v, less than about 0.081% w/v, less than about 0.08% w/v, less than about 0.079% w/v, less than about 0.078% w/v, less than about 0.077% w/v, less than about 0.076% w/v, less than about 0.075% w/v, less than about 0.074% w/v, less than about 0.073% w/v, less than about 0.072% w/v, less than about 0.071% w/v, less than about 0.07% w/v, less than about 0.069% w/v, less than about 0.068% w/v, less than about 0.067% w/v, less than about 0.066% w/v, less than about 0.065% w/v, less than about 0.064% w/v, less than about 0.063% w/v, less than about 0.062% w/v, less than about 0.061% w/v, less than about 0.06% w/v, less than about 0.059% w/v, less than about 0.058% w/v, less than about 0.057% w/v, less than about 0.056% w/v, less than about 0.055% w/v, less than about 0.054% w/v, less than about 0.053% w/v, less than about 0.052% w/v, less than about 0.051% w/v, less than about 0.05% w/v, less than about 0.049% w/v, less than about 0.048% w/v, less than about 0.047% w/v, less than about 0.046% w/v, less than about 0.045% w/v, less than about 0.044% w/v, less than about 0.043% w/v, less than about 0.042% w/v, less than about 0.041% w/v, less than about 0.04% w/v, less than about 0.039% w/v, less than about 0.038% w/v, less than about 0.037% w/v, less than about 0.036% w/v, less than about 0.035% w/v, less than about 0.034% w/v, less than about 0.033% w/v, less than about 0.032% w/v, less than about 0.031% w/v, less than about 0.03% w/v, less than about 0.029% w/v, less than about 0.028% w/v, less than about 0.027% w/v, less than about 0.026% w/v, less than about 0.025% w/v, less than about 0.024% w/v, less than about 0.023% w/v, less than about 0.022% w/v, less than about 0.021% w/v, less than about 0.02% w/v, less than about 0.019% w/v, less than about 0.018% w/v, less than about 0.017% w/v, less than about 0.016% w/v, less than about 0.015% w/v, less than about 0.014% w/v, less than about 0.013% w/v, less than about 0.012% w/v, less than about 0.011% w/v, or less than about 0.01% w/v.

In some embodiments, the aqueous-based composition contains phenylephrine, or a pharmaceutically acceptable salt, ester, hydrate, or solvate thereof, in an amount that is greater than about 0.01% w/v, greater than about 0.011% w/v, greater than about 0.012% w/v, greater than about 0.013% w/v, greater than about 0.014% w/v, greater than about 0.015% w/v, greater than about 0.016% w/v, greater than about 0.017% w/v, greater than about 0.018% w/v, greater than about 0.019% w/v, greater than about 0.02% w/v, greater than about 0.021% w/v, greater than about 0.022% w/v, greater than about 0.023% w/v, greater than about 0.024% w/v, greater than about 0.025% w/v, greater than about 0.026% w/v, greater than about 0.027% w/v, greater than about 0.028% w/v, greater than about 0.029% w/v, greater than about 0.03% w/v, greater than about 0.031% w/v, greater than about 0.032% w/v, greater than about 0.033% w/v, greater than about 0.034% w/v, greater than about 0.035% w/v, greater than about 0.036% w/v, greater than about 0.037% w/v, greater than about 0.038% w/v, greater than about 0.039% w/v, greater than about 0.04% w/v, greater than about 0.041% w/v, greater than about 0.042% w/v, greater than about 0.043% w/v, greater than about 0.044% w/v, greater than about 0.045% w/v, greater than about 0.046% w/v, greater than about 0.047% w/v, greater than about 0.048% w/v, greater than about 0.049% w/v, or greater than about 0.05% w/v, and dextromethorphan, or a pharmaceutically acceptable salt, ester, hydrate, or solvate thereof, in an amount that is greater than about 0.01% w/v, greater than about 0.011% w/v, greater than about 0.012% w/v, greater than about 0.013% w/v, greater than about 0.014% w/v, greater than about 0.015% w/v, greater than about 0.016% w/v, greater than about 0.017% w/v, greater than about 0.018% w/v, greater than about 0.019% w/v, greater than about 0.02% w/v, greater than about 0.021% w/v, greater than about 0.022% w/v, greater than about 0.023% w/v, greater than about 0.024% w/v, greater than about 0.025% w/v, greater than about 0.026% w/v, greater than about 0.027% w/v, greater than about 0.028% w/v, greater than about 0.029% w/v, greater than about 0.03% w/v, greater than about 0.031% w/v, greater than about 0.032% w/v, greater than about 0.033% w/v, greater than about 0.034% w/v, greater than about 0.035% w/v, greater than about 0.036% w/v, greater than about 0.037% w/v, greater than about 0.038% w/v, greater than about 0.039% w/v, greater than about 0.04% w/v, greater than about 0.041% w/v, greater than about 0.042% w/v, greater than about 0.043% w/v, greater than about 0.044% w/v, greater than about 0.045% w/v, greater than about 0.046% w/v, greater than about 0.047% w/v, greater than about 0.048% w/v, greater than about 0.049% w/v, greater than about 0.05% w/v, greater than about 0.051% w/v, greater than about 0.052% w/v, greater than about 0.053% w/v, greater than about 0.054% w/v, greater than about 0.055% w/v, greater than about 0.056% w/v, greater than about 0.057% w/v, greater than about 0.058% w/v, greater than about 0.059% w/v, greater than about 0.06% w/v, greater than about 0.061% w/v, greater than about 0.062% w/v, greater than about 0.063% w/v, greater than about 0.064% w/v, greater than about 0.065% w/v, greater than about 0.066% w/v, greater than about 0.067% w/v, greater than about 0.068% w/v, greater than about 0.069% w/v, greater than about 0.07% w/v, greater than about 0.071% w/v, greater than about 0.072% w/v, greater than about 0.073% w/v, greater than about 0.074% w/v, greater than about 0.075% w/v, greater than about 0.076% w/v, greater than about 0.077% w/v, greater than about 0.078% w/v, greater than about 0.079% w/v, greater than about 0.08% w/v, greater than about 0.081% w/v, greater than about 0.082% w/v, greater than about 0.083% w/v, greater than about 0.084% w/v, greater than about 0.085% w/v, greater than about 0.086% w/v, greater than about 0.087% w/v, greater than about 0.088% w/v, greater than about 0.089% w/v, greater than about 0.09% w/v, greater than about 0.091% w/v, greater than about 0.092% w/v, greater than about 0.093% w/v, greater than about 0.094% w/v, greater than about 0.095% w/v, greater than about 0.096% w/v, greater than about 0.097% w/v, greater than about 0.098% w/v, greater than about 0.099% w/v, or greater than about 0.1% w/v.

In some embodiments, the aqueous-based composition contains phenylephrine, or a pharmaceutically acceptable salt, ester, hydrate, or solvate thereof, in an amount that has a range of % w/v having an upper limit of about 0.05% w/v, 0.04% w/v, 0.03% w/v, 0.02% w/v, or 0.01% w/v, and an independently selected lower limit of about 0.01% w/v, 0.02% w/v, 0.03% w/v, 0.04% w/v, or 0.05% w/v, where the lower limit is less than the upper limit, and dextromethorphan, or a pharmaceutically acceptable salt, ester, hydrate, or solvate thereof, in an amount that has a range of % w/v having an upper limit of about 0.1% w/v, 0.09% w/v, 0.08% w/v, 0.07% w/v, 0.06% w/v, 0.05% w/v, 0.04% w/v, 0.03% w/v, 0.02% w/v, or 0.01% w/v, and an independently selected lower limit of about 0.01% w/v, 0.02% w/v, 0.03% w/v, 0.04% w/v, 0.05% w/v, 0.06% w/v, 0.07% w/v, 0.08% w/v, 0.09% w/v, or 0.1% w/v, where the lower limit is less than the upper limit. In some embodiments, the aqueous-based composition contains phenylephrine, or a pharmaceutically acceptable salt, ester, hydrate, or solvate thereof, in an amount of about 0.01% w/v, about 0.02% w/v, about 0.03% w/v, about 0.04% w/v, or about 0.05% w/v, and dextromethorphan, or a pharmaceutically acceptable salt, ester, hydrate, or solvate thereof, in an amount of about 0.01% w/v, about 0.02% w/v, about 0.03% w/v, about 0.04% w/v, about 0.05% w/v, about 0.06% w/v, about 0.07% w/v, about 0.08% w/v, about 0.09% w/v, or about 0.1% w/v. In some embodiments, the aqueous-based composition contains phenylephrine, or a pharmaceutically acceptable salt, ester, hydrate, or solvate thereof, in an amount of about 0.05% w/v, and dextromethorphan, or a pharmaceutically acceptable salt, ester, hydrate, or solvate thereof, in an amount of about 0.1% w/v.

In some embodiments, the aqueous-based composition contains phenylephrine, or a pharmaceutically acceptable salt, ester, hydrate, or solvate thereof (e.g., phenylephrine HCl, phenylephrine bitartrate, etc.) in an amount that ranges from 1 mg/ml to 10 mg/ml and dextromethorphan, or a pharmaceutically acceptable salt, ester, hydrate, or solvate thereof (e.g., dextromethorphan HBr, etc.) in an amount that ranges from 2 mg/ml to 3 mg/ml. In some embodiments, the aqueous-based composition contains phenylephrine, or a pharmaceutically acceptable salt, ester, hydrate, or solvate thereof, in an amount that is less than about 10 mg/ml, less than about 9.9 mg/ml, less than about 9.8 mg/ml, less than about 9.7 mg/ml, less than about 9.6 mg/ml, less than about 9.5 mg/ml, less than about 9.4 mg/ml, less than about 9.3 mg/ml, less than about 9.2 mg/ml, less than about 9.1 mg/ml, less than about 9 mg/ml, less than about 8.9 mg/ml, less than about 8.8 mg/ml, less than about 8.7 mg/ml, less than about 8.6 mg/ml, less than about 8.5 mg/ml, less than about 8.4 mg/ml, less than about 8.3 mg/ml, less than about 8.2 mg/ml, less than about 8.1 mg/ml, less than about 8 mg/ml, less than about 7.9 mg/ml, less than about 7.8 mg/ml, less than about 7.7 mg/ml, less than about 7.6 mg/ml, less than about 7.5 mg/ml, less than about 7.4 mg/ml, less than about 7.3 mg/ml, less than about 7.2 mg/ml, less than about 7.1 mg/ml, less than about 7 mg/ml, less than about 6.9 mg/ml, less than about 6.8 mg/ml, less than about 6.7 mg/ml, less than about 6.6 mg/ml, less than about 6.5 mg/ml, less than about 6.4 mg/ml, less than about 6.3 mg/ml, less than about 6.2 mg/ml, less than about 6.1 mg/ml, less than about 6 mg/ml, less than about 5.9 mg/ml, less than about 5.8 mg/ml, less than about 5.7 mg/ml, less than about 5.6 mg/ml, less than about 5.5 mg/ml, less than about 5.4 mg/ml, less than about 5.3 mg/ml, less than about 5.2 mg/ml, less than about 5.1 mg/ml, less than about 5 mg/ml, less than about 4.9 mg/ml, less than about 4.8 mg/ml, less than about 4.7 mg/ml, less than about 4.6 mg/ml, less than about 4.5 mg/ml, less than about 4.4 mg/ml, less than about 4.3 mg/ml, less than about 4.2 mg/ml, less than about 4.1 mg/ml, less than about 4 mg/ml, less than about 3.9 mg/ml, less than about 3.8 mg/ml, less than about 3.7 mg/ml, less than about 3.6 mg/ml, less than about 3.5 mg/ml, less than about 3.4 mg/ml, less than about 3.3 mg/ml, less than about 3.2 mg/ml, less than about 3.1 mg/ml, less than about 3 mg/ml, less than about 2.9 mg/ml, less than about 2.8 mg/ml, less than about 2.7 mg/ml, less than about 2.6 mg/ml, less than about 2.5 mg/ml, less than about 2.4 mg/ml, less than about 2.3 mg/ml, less than about 2.2 mg/ml, less than about 2.1 mg/ml, less than about 2 mg/ml, less than about 1.9 mg/ml, less than about 1.8 mg/ml, less than about 1.7 mg/ml, less than about 1.6 mg/ml, less than about 1.5 mg/ml, less than about 1.4 mg/ml, less than about 1.3 mg/ml, less than about 1.2 mg/ml, less than about 1.1 mg/ml, or less than about 1 mg/ml, and dextromethorphan, or a pharmaceutically acceptable salt, ester, hydrate, or solvate thereof, in an amount that is less than about 3 mg/ml, less than about 2.99 mg/ml, less than about 2.98 mg/ml, less than about 2.97 mg/ml, less than about 2.96 mg/ml, less than about 2.95 mg/ml, less than about 2.94 mg/ml, less than about 2.93 mg/ml, less than about 2.92 mg/ml, less than about 2.91 mg/ml, less than about 2.9 mg/ml, less than about 2.89 mg/ml, less than about 2.88 mg/ml, less than about 2.87 mg/ml, less than about 2.86 mg/ml, less than about 2.85 mg/ml, less than about 2.84 mg/ml, less than about 2.83 mg/ml, less than about 2.82 mg/ml, less than about 2.81 mg/ml, less than about 2.8 mg/ml, less than about 2.79 mg/ml, less than about 2.78 mg/ml, less than about 2.77 mg/ml, less than about 2.76 mg/ml, less than about 2.75 mg/ml, less than about 2.74 mg/ml, less than about 2.73 mg/ml, less than about 2.72 mg/ml, less than about 2.71 mg/ml, less than about 2.7 mg/ml, less than about 2.69 mg/ml, less than about 2.68 mg/ml, less than about 2.67 mg/ml, less than about 2.66 mg/ml, less than about 2.65 mg/ml, less than about 2.64 mg/ml, less than about 2.63 mg/ml, less than about 2.62 mg/ml, less than about 2.61 mg/ml, less than about 2.6 mg/ml, less than about 2.59 mg/ml, less than about 2.58 mg/ml, less than about 2.57 mg/ml, less than about 2.56 mg/ml, less than about 2.55 mg/ml, less than about 2.54 mg/ml, less than about 2.53 mg/ml, less than about 2.52 mg/ml, less than about 2.51 mg/ml, less than about 2.5 mg/ml, less than about 2.49 mg/ml, less than about 2.48 mg/ml, less than about 2.47 mg/ml, less than about 2.46 mg/ml, less than about 2.45 mg/ml, less than about 2.44 mg/ml, less than about 2.43 mg/ml, less than about 2.42 mg/ml, less than about 2.41 mg/ml, less than about 2.4 mg/ml, less than about 2.39 mg/ml, less than about 2.38 mg/ml, less than about 2.37 mg/ml, less than about 2.36 mg/ml, less than about 2.35 mg/ml, less than about 2.34 mg/ml, less than about 2.33 mg/ml, less than about 2.32 mg/ml, less than about 2.31 mg/ml, less than about 2.3 mg/ml, less than about 2.29 mg/ml, less than about 2.28 mg/ml, less than about 2.27 mg/ml, less than about 2.26 mg/ml, less than about 2.25 mg/ml, less than about 2.24 mg/ml, less than about 2.23 mg/ml, less than about 2.22 mg/ml, less than about 2.21 mg/ml, less than about 2.2 mg/ml, less than about 2.19 mg/ml, less than about 2.18 mg/ml, less than about 2.17 mg/ml, less than about 2.16 mg/ml, less than about 2.15 mg/ml, less than about 2.14 mg/ml, less than about 2.13 mg/ml, less than about 2.12 mg/ml, less than about 2.11 mg/ml, or less than about 2 mg/ml.

In some embodiments, the aqueous-based composition contains phenylephrine, or a pharmaceutically acceptable salt, ester, hydrate, or solvate thereof, in an amount that is greater than about 1 mg/ml, greater than about 1.1 mg/ml, greater than about 1.2 mg/ml, greater than about 1.3 mg/ml, greater than about 1.4 mg/ml, greater than about 1.5 mg/ml, greater than about 1.6 mg/ml, greater than about 1.7 mg/ml, greater than about 1.8 mg/ml, greater than about 1.9 mg/ml, greater than about 2 mg/ml, greater than about 2.1 mg/ml, greater than about 2.2 mg/ml, greater than about 2.3 mg/ml, greater than about 2.4 mg/ml, greater than about 2.5 mg/ml, greater than about 2.6 mg/ml, greater than about 2.7 mg/ml, greater than about 2.8 mg/ml, greater than about 2.9 mg/ml, greater than about 3 mg/ml, greater than about 3.1 mg/ml, greater than about 3.2 mg/ml, greater than about 3.3 mg/ml, greater than about 3.4 mg/ml, greater than about 3.5 mg/ml, greater than about 3.6 mg/ml, greater than about 3.7 mg/ml, greater than about 3.8 mg/ml, greater than about 3.9 mg/ml, greater than about 4 mg/ml, greater than about 4.1 mg/ml, greater than about 4.2 mg/ml, greater than about 4.3 mg/ml, greater than about 4.4 mg/ml, greater than about 4.5 mg/ml, greater than about 4.6 mg/ml, greater than about 4.7 mg/ml, greater than about 4.8 mg/ml, greater than about 4.9 mg/ml, greater than about 5 mg/ml, greater than about 5.1 mg/ml, greater than about 5.2 mg/ml, greater than about 5.3 mg/ml, greater than about 5.4 mg/ml, greater than about 5.5 mg/ml, greater than about 5.6 mg/ml, greater than about 5.7 mg/ml, greater than about 5.8 mg/ml, greater than about 5.9 mg/ml, greater than about 6 mg/ml, greater than about 6.1 mg/ml, greater than about 6.2 mg/ml, greater than about 6.3 mg/ml, greater than about 6.4 mg/ml, greater than about 6.5 mg/ml, greater than about 6.6 mg/ml, greater than about 6.7 mg/ml, greater than about 6.8 mg/ml, greater than about 6.9 mg/ml, greater than about 7 mg/ml, greater than about 7.1 mg/ml, greater than about 7.2 mg/ml, greater than about 7.3 mg/ml, greater than about 7.4 mg/ml, greater than about 7.5 mg/ml, greater than about 7.6 mg/ml, greater than about 7.7 mg/ml w/v, greater than about 7.8 mg/ml, greater than about 7.9 mg/ml, greater than about 8 mg/ml, greater than about 8.1 mg/ml, greater than about 8.2 mg/ml, greater than about 8.3 mg/ml, greater than about 8.4 mg/ml, greater than about 8.5 mg/ml, greater than about 8.6 mg/ml, greater than about 8.7 mg/ml, greater than about 8.8 mg/ml, greater than about 8.9 mg/ml, greater than about 9 mg/ml, greater than about 9.1 mg/ml, greater than about 9.2 mg/ml, greater than about 9.3 mg/ml, greater than about 9.4 mg/ml, greater than about 9.5 mg/ml, greater than about 9.6 mg/ml, greater than about 9.7 mg/ml, greater than about 9.8 mg/ml, greater than about 9.9 mg/ml, or greater than about 10 mg/ml, and dextromethorphan, or a pharmaceutically acceptable salt, ester, hydrate, or solvate thereof, in an amount that is greater than about 2 mg/ml, greater than about 2.11 mg/ml, greater than about 2.12 mg/ml, greater than about 2.13 mg/ml, greater than about 2.14 mg/ml, greater than about 2.15 mg/ml, greater than about 2.16 mg/ml, greater than about 2.17 mg/ml, greater than about 2.18 mg/ml, greater than about 2.19 mg/ml, greater than about 2.2 mg/ml, greater than about 2.21 mg/ml, greater than about 2.22 mg/ml, greater than about 2.23 mg/ml, greater than about 2.24 mg/ml, greater than about 2.25 mg/ml, greater than about 2.26 mg/ml, greater than about 2.27 mg/ml, greater than about 2.28 mg/ml, greater than about 2.29 mg/ml, greater than about 2.3 mg/ml, greater than about 2.31 mg/ml, greater than about 2.32 mg/ml, greater than about 2.33 mg/ml, greater than about 2.34 mg/ml, greater than about 2.35 mg/ml, greater than about 2.36 mg/ml, greater than about 2.37 mg/ml, greater than about 2.38 mg/ml, greater than about 2.39 mg/ml, greater than about 2.4 mg/ml, greater than about 2.41 mg/ml, greater than about 2.42 mg/ml, greater than about 2.43 mg/ml, greater than about 2.44 mg/ml, greater than about 2.45 mg/ml, greater than about 2.46 mg/ml, greater than about 2.47 mg/ml, greater than about 2.48 mg/ml, greater than about 2.49 mg/ml, greater than about 2.5 mg/ml, greater than about 2.51 mg/ml, greater than about 2.52 mg/ml, greater than about 2.53 mg/ml, greater than about 2.54 mg/ml, greater than about 2.55 mg/ml, greater than about 2.56 mg/ml, greater than about 2.57 mg/ml, greater than about 2.58 mg/ml, greater than about 2.59 mg/ml, greater than about 2.6 mg/ml, greater than about 2.61 mg/ml, greater than about 2.62 mg/ml, greater than about 2.63 mg/ml, greater than about 2.64 mg/ml, greater than about 2.65 mg/ml, greater than about 2.66 mg/ml, greater than about 2.67 mg/ml, greater than about 2.68 mg/ml, greater than about 2.69 mg/ml, greater than about 2.7 mg/ml, greater than about 2.71 mg/ml, greater than about 2.72 mg/ml, greater than about 2.73 mg/ml, greater than about 2.74 mg/ml, greater than about 2.75 mg/ml, greater than about 2.76 mg/ml, greater than about 2.77 mg/ml w/v, greater than about 2.78 mg/ml, greater than about 2.79 mg/ml, greater than about 2.8 mg/ml, greater than about 2.81 mg/ml, greater than about 2.82 mg/ml, greater than about 2.83 mg/ml, greater than about 2.84 mg/ml, greater than about 2.85 mg/ml, greater than about 2.86 mg/ml, greater than about 2.87 mg/ml, greater than about 2.88 mg/ml, greater than about 2.89 mg/ml, greater than about 2.9 mg/ml, greater than about 2.91 mg/ml, greater than about 2.92 mg/ml, greater than about 2.93 mg/ml, greater than about 2.94 mg/ml, greater than about 2.95 mg/ml, greater than about 2.96 mg/ml, greater than about 2.97 mg/ml, greater than about 2.98 mg/ml, greater than about 2.99 mg/ml, or greater than about 3 mg/ml.

In some embodiments, the aqueous-based composition contains phenylephrine, or a pharmaceutically acceptable salt, ester, hydrate, or solvate thereof, in an amount that has a range of mg/ml having an upper limit of about 10 mg/ml, 9 mg/ml, 8 mg/ml, 7 mg/ml, 6 mg/ml, 5 mg/ml, 4 mg/ml, 3 mg/ml, 2 mg/ml, or 1 mg/ml, and an independently selected lower limit of about 1 mg/ml, 2 mg/ml, 3 mg/ml, 4 mg/ml, 5 mg/ml, 6 mg/ml, 7 mg/ml, 8 mg/ml, 9 mg/ml, or 10 mg/ml, where the lower limit is less than the upper limit, and dextromethorphan, or a pharmaceutically acceptable salt, ester, hydrate, or solvate thereof, in an amount that has a range of mg/ml having an upper limit of about 3 mg/ml, 2.9 mg/ml, 2.8 mg/ml, 2.7 mg/ml, 2.6 mg/ml, 2.5 mg/ml, 2.4 mg/ml, 2.3 mg/ml, 2.2 mg/ml, 2.1 mg/ml, or 2 mg/ml and an independently selected lower limit of about 2 mg/ml, 2.1 mg/ml, 2.2 mg/ml, 2.3 mg/ml, 2.4 mg/ml, 2.5 mg/ml, 2.6 mg/ml, 2.7 mg/ml, 2.8 mg/ml, 2.9 mg/ml, or 3 mg/ml, where the lower limit is less than the upper limit. In some embodiments, the aqueous-based composition contains phenylephrine, or a pharmaceutically acceptable salt, ester, hydrate, or solvate thereof, in an amount of about 1 mg/ml, about 2 mg/ml, about 3 mg/ml, about 4 mg/ml, about 5 mg/ml, about 6 mg/ml, about 7 mg/ml, about 8 mg/ml, about 9 mg/ml, or about 10 mg/ml, and dextromethorphan, or a pharmaceutically acceptable salt, ester, hydrate, or solvate thereof, in an amount of about 2 mg/ml, about 2.1 mg/ml, about 2.2 mg/ml, about 2.3 mg/ml, about 2.4 mg/ml, about 2.5 mg/ml, about 2.6 mg/ml, about 2.7 mg/ml, about 2.8 mg/ml, about 2.9 mg/ml, or about 3 mg/ml.

In some embodiments, the two APIs are dextromethorphan, or a pharmaceutically acceptable salt, ester, hydrate, or solvate thereof (e.g., dextromethorphan HBr, etc.), and chlorpheniramine, or a pharmaceutically acceptable salt, ester, hydrate, or solvate thereof (e.g., chlorpheniramine maleate, etc.). In some embodiments, the aqueous-based composition contains dextromethorphan, or a pharmaceutically acceptable salt, ester, hydrate, or solvate thereof in an amount that ranges from in an amount that ranges from 2 mg/ml to 3 mg/ml and chlorpheniramine, or a pharmaceutically acceptable salt, ester, hydrate, or solvate thereof in an amount that ranges from 0.2 mg/ml to 100 mg/ml. In some embodiments, the aqueous-based composition contains dextromethorphan, or a pharmaceutically acceptable salt, ester, hydrate, or solvate thereof, in an amount that is less than about 3 mg/ml, less than about 2.99 mg/ml, less than about 2.98 mg/ml, less than about 2.97 mg/ml, less than about 2.96 mg/ml, less than about 2.95 mg/ml, less than about 2.94 mg/ml, less than about 2.93 mg/ml, less than about 2.92 mg/ml, less than about 2.91 mg/ml, less than about 2.9 mg/ml, less than about 2.89 mg/ml, less than about 2.88 mg/ml, less than about 2.87 mg/ml, less than about 2.86 mg/ml, less than about 2.85 mg/ml, less than about 2.84 mg/ml, less than about 2.83 mg/ml, less than about 2.82 mg/ml, less than about 2.81 mg/ml, less than about 2.8 mg/ml, less than about 2.79 mg/ml, less than about 2.78 mg/ml, less than about 2.77 mg/ml, less than about 2.76 mg/ml, less than about 2.75 mg/ml, less than about 2.74 mg/ml, less than about 2.73 mg/ml, less than about 2.72 mg/ml, less than about 2.71 mg/ml, less than about 2.7 mg/ml, less than about 2.69 mg/ml, less than about 2.68 mg/ml, less than about 2.67 mg/ml, less than about 2.66 mg/ml, less than about 2.65 mg/ml, less than about 2.64 mg/ml, less than about 2.63 mg/ml, less than about 2.62 mg/ml, less than about 2.61 mg/ml, less than about 2.6 mg/ml, less than about 2.59 mg/ml, less than about 2.58 mg/ml, less than about 2.57 mg/ml, less than about 2.56 mg/ml, less than about 2.55 mg/ml, less than about 2.54 mg/ml, less than about 2.53 mg/ml, less than about 2.52 mg/ml, less than about 2.51 mg/ml, less than about 2.5 mg/ml, less than about 2.49 mg/ml, less than about 2.48 mg/ml, less than about 2.47 mg/ml, less than about 2.46 mg/ml, less than about 2.45 mg/ml, less than about 2.44 mg/ml, less than about 2.43 mg/ml, less than about 2.42 mg/ml, less than about 2.41 mg/ml, less than about 2.4 mg/ml, less than about 2.39 mg/ml, less than about 2.38 mg/ml, less than about 2.37 mg/ml, less than about 2.36 mg/ml, less than about 2.35 mg/ml, less than about 2.34 mg/ml, less than about 2.33 mg/ml, less than about 2.32 mg/ml, less than about 2.31 mg/ml, less than about 2.3 mg/ml, less than about 2.29 mg/ml, less than about 2.28 mg/ml, less than about 2.27 mg/ml, less than about 2.26 mg/ml, less than about 2.25 mg/ml, less than about 2.24 mg/ml, less than about 2.23 mg/ml, less than about 2.22 mg/ml, less than about 2.21 mg/ml, less than about 2.2 mg/ml, less than about 2.19 mg/ml, less than about 2.18 mg/ml, less than about 2.17 mg/ml, less than about 2.16 mg/ml, less than about 2.15 mg/ml, less than about 2.14 mg/ml, less than about 2.13 mg/ml, less than about 2.12 mg/ml, less than about 2.11 mg/ml, or less than about 2 mg/ml, and chlorpheniramine, or a pharmaceutically acceptable salt, ester, hydrate, or solvate thereof, in an amount that is less than about 100 mg/ml, less than about 95 mg/ml, less than about 90 mg/ml, less than about 85 mg/ml, less than about 80 mg/ml, less than about 75 mg/ml, less than about 70 mg/ml, less than about 65 mg/ml, less than about 60 mg/ml, less than about 55 mg/ml, less than about 50 mg/ml, less than about 45 mg/ml, less than about 40 mg/ml, less than about 35 mg/ml, less than about 30 mg/ml, less than about 25 mg/ml, less than about 20 mg/ml, less than about 15 mg/ml, less than about 10 mg/ml less than about 9 mg/ml, less than about 8 mg/ml, less than about 7 mg/ml, less than about 6 mg/ml, less than about 5 mg/ml, less than about 4 mg/ml, less than about 3 mg/ml, less than about 2 mg/ml, less than about 1 mg/ml, less than about 0.9 mg/ml, less than about 0.8 mg/ml, less than about 0.7 mg/ml, less than about 0.6 mg/ml, less than about 0.5 mg/ml, less than about 0.4 mg/ml, less than about 0.3 mg/ml, or less than about 0.2 mg/ml.

In some embodiments, the aqueous-based composition contains dextromethorphan, or a pharmaceutically acceptable salt, ester, hydrate, or solvate thereof, in an amount that is greater than about 2 mg/ml, greater than about 2.11 mg/ml, greater than about 2.12 mg/ml, greater than about 2.13 mg/ml, greater than about 2.14 mg/ml, greater than about 2.15 mg/ml, greater than about 2.16 mg/ml, greater than about 2.17 mg/ml, greater than about 2.18 mg/ml, greater than about 2.19 mg/ml, greater than about 2.2 mg/ml, greater than about 2.21 mg/ml, greater than about 2.22 mg/ml, greater than about 2.23 mg/ml, greater than about 2.24 mg/ml, greater than about 2.25 mg/ml, greater than about 2.26 mg/ml, greater than about 2.27 mg/ml, greater than about 2.28 mg/ml, greater than about 2.29 mg/ml, greater than about 2.3 mg/ml, greater than about 2.31 mg/ml, greater than about 2.32 mg/ml, greater than about 2.33 mg/ml, greater than about 2.34 mg/ml, greater than about 2.35 mg/ml, greater than about 2.36 mg/ml, greater than about 2.37 mg/ml, greater than about 2.38 mg/ml, greater than about 2.39 mg/ml, greater than about 2.4 mg/ml, greater than about 2.41 mg/ml, greater than about 2.42 mg/ml, greater than about 2.43 mg/ml, greater than about 2.44 mg/ml, greater than about 2.45 mg/ml, greater than about 2.46 mg/ml, greater than about 2.47 mg/ml, greater than about 2.48 mg/ml, greater than about 2.49 mg/ml, greater than about 2.5 mg/ml, greater than about 2.51 mg/ml, greater than about 2.52 mg/ml, greater than about 2.53 mg/ml, greater than about 2.54 mg/ml, greater than about 2.55 mg/ml, greater than about 2.56 mg/ml, greater than about 2.57 mg/ml, greater than about 2.58 mg/ml, greater than about 2.59 mg/ml, greater than about 2.6 mg/ml, greater than about 2.61 mg/ml, greater than about 2.62 mg/ml, greater than about 2.63 mg/ml, greater than about 2.64 mg/ml, greater than about 2.65 mg/ml, greater than about 2.66 mg/ml, greater than about 2.67 mg/ml, greater than about 2.68 mg/ml, greater than about 2.69 mg/ml, greater than about 2.7 mg/ml, greater than about 2.71 mg/ml, greater than about 2.72 mg/ml, greater than about 2.73 mg/ml, greater than about 2.74 mg/ml, greater than about 2.75 mg/ml, greater than about 2.76 mg/ml, greater than about 2.77 mg/ml w/v, greater than about 2.78 mg/ml, greater than about 2.79 mg/ml, greater than about 2.8 mg/ml, greater than about 2.81 mg/ml, greater than about 2.82 mg/ml, greater than about 2.83 mg/ml, greater than about 2.84 mg/ml, greater than about 2.85 mg/ml, greater than about 2.86 mg/ml, greater than about 2.87 mg/ml, greater than about 2.88 mg/ml, greater than about 2.89 mg/ml, greater than about 2.9 mg/ml, greater than about 2.91 mg/ml, greater than about 2.92 mg/ml, greater than about 2.93 mg/ml, greater than about 2.94 mg/ml, greater than about 2.95 mg/ml, greater than about 2.96 mg/ml, greater than about 2.97 mg/ml, greater than about 2.98 mg/ml, greater than about 2.99 mg/ml, or greater than about 3 mg/ml, and chlorpheniramine, or a pharmaceutically acceptable salt, ester, hydrate, or solvate thereof, in an amount that is greater than about 0.2 mg/ml, greater than about 0.3 mg/ml, greater than about 0.4 mg/ml, greater than about 0.5 mg/ml, greater than about 0.6 mg/ml, greater than about 0.7 mg/ml, greater than about 0.8 mg/ml, greater than about 0.9 mg/ml, greater than about 1 mg/ml, greater than about 2 mg/ml, greater than about 3 mg/ml, greater than about 4 mg/ml, greater than about 5 mg/ml, greater than about 6 mg/ml, greater than about 7 mg/ml, greater than about 8 mg/ml, greater than about 9 mg/ml, greater than about 10 mg/ml, greater than about 15 mg/ml, greater than about 20 mg/ml, greater than about 25 mg/ml, greater than about 30 mg/ml, greater than about 35 mg/ml, greater than about 40 mg/ml, greater than about 45 mg/ml, greater than about 50 mg/ml, greater than about 55 mg/ml, greater than about 60 mg/ml, greater than about 65 mg/ml, greater than about 70 mg/ml, greater than about 75 mg/ml, greater than about 80 mg/ml, greater than about 85 mg/ml, greater than about 90 mg/ml, greater than about 95 mg/ml, or greater than about 100 mg/ml.

In some embodiments, the aqueous-based composition contains dextromethorphan, or a pharmaceutically acceptable salt, ester, hydrate, or solvate thereof, in an amount that has a range of mg/ml having an upper limit of about 3 mg/ml, 2.9 mg/ml, 2.8 mg/ml, 2.7 mg/ml, 2.6 mg/ml, 2.5 mg/ml, 2.4 mg/ml, 2.3 mg/ml, 2.2 mg/ml, 2.1 mg/ml, or 2 mg/ml and an independently selected lower limit of about 2 mg/ml, 2.1 mg/ml, 2.2 mg/ml, 2.3 mg/ml, 2.4 mg/ml, or 2.5 mg/ml, 2.6 mg/ml, 2.7 mg/ml, 2.8 mg/ml, 2.9 mg/ml, or 3 mg/ml, where the lower limit is less than the upper limit, and chlorpheniramine, or a pharmaceutically acceptable salt, ester, hydrate, or solvate thereof, in an amount that has a range of mg/ml having an upper limit of about 100 mg/ml, 80 mg/ml, 60 mg/ml, 40 mg/ml, 20 mg/ml, 10 mg/ml, 1 mg/ml, 0.8 mg/ml, 0.4 mg/ml, or 0.2 mg/ml and an independently selected lower limit of about 0.2 mg/ml, 0.4 mg/ml, 0.8 mg/ml, 1 mg/ml, 10 mg/ml, 20 mg/ml, 30 mg/ml, 40 mg/ml, 50 mg/ml, 60 mg/ml, 70 mg/ml, 80 mg/ml, 90 mg/ml, or 100 mg/ml, where the lower limit is less than the upper limit. In some embodiments, the aqueous-based composition contains dextromethorphan, or a pharmaceutically acceptable salt, ester, hydrate, or solvate thereof, in an amount of about 2 mg/ml, about 2.1 mg/ml, about 2.2 mg/ml, about 2.3 mg/ml, about 2.4 mg/ml, about 2.5 mg/ml, about 2.6 mg/ml, about 2.7 mg/ml, about 2.8 mg/ml, about 2.9 mg/ml, or about 3 mg/ml, and chlorpheniramine, or a pharmaceutically acceptable salt, ester, hydrate, or solvate thereof, in an amount of about 0.2 mg/ml, about 0.4 mg/ml, about 0.8 mg/ml, about 1 mg/ml, about 10 mg/ml, about 20 mg/ml, about 30 mg/ml, about 40 mg/ml, about 50 mg/ml, about 60 mg/ml, about 70 mg/ml, about 80 mg/ml, about 90 mg/ml, or about 100 mg/ml.

In some embodiments, the aqueous-based composition contains dextromethorphan, or a pharmaceutically acceptable salt, ester, hydrate, or solvate thereof in an amount that ranges from 0.01% w/v to about 0.10% w/v and chlorpheniramine, or a pharmaceutically acceptable salt, ester, hydrate, or solvate thereof (e.g., chlorpheniramine maleate, etc.) in an amount that ranges from 0.01% w/v to about 0.05% w/v. In some embodiments, the aqueous-based composition contains dextromethorphan, or a pharmaceutically acceptable salt, ester, hydrate, or thereof (e.g., dextromethorphan HBr, etc.) in an amount of about 0.1% w/v, and chlorpheniramine, or a pharmaceutically acceptable salt, ester, hydrate, or solvate thereof (e.g., chlorpheniramine maleate, etc.) in an amount of about 0.02% w/v.

In some embodiments, the two APIs are phenylephrine, or a pharmaceutically acceptable salt, ester, hydrate, or solvate thereof (e.g., phenylephrine HCl, etc.), and chlorpheniramine, or a pharmaceutically acceptable salt, ester, hydrate, or solvate thereof (e.g., chlorpheniramine maleate, etc.). In some embodiments, the aqueous-based composition contains phenylephrine, or a pharmaceutically acceptable salt, ester, hydrate, or solvate thereof in an amount that ranges from 1 mg/ml to about 10 mg/ml and chlorpheniramine, or a pharmaceutically acceptable salt, ester, hydrate, or solvate thereof in an amount that ranges from 0.2 mg/ml to 100 mg/ml. In some embodiments, the aqueous-based composition contains phenylephrine, or a pharmaceutically acceptable salt, ester, hydrate, or solvate thereof, in an amount that is less than about 10 mg/ml, less than about 9.9 mg/ml, less than about 9.8 mg/ml, less than about 9.7 mg/ml, less than about 9.6 mg/ml, less than about 9.5 mg/ml, less than about 9.4 mg/ml, less than about 9.3 mg/ml, less than about 9.2 mg/ml, less than about 9.1 mg/ml, less than about 9 mg/ml, less than about 8.9 mg/ml, less than about 8.8 mg/ml, less than about 8.7 mg/ml, less than about 8.6 mg/ml, less than about 8.5 mg/ml, less than about 8.4 mg/ml, less than about 8.3 mg/ml, less than about 8.2 mg/ml, less than about 8.1 mg/ml, less than about 8 mg/ml, less than about 7.9 mg/ml, less than about 7.8 mg/ml, less than about 7.7 mg/ml, less than about 7.6 mg/ml, less than about 7.5 mg/ml, less than about 7.4 mg/ml, less than about 7.3 mg/ml, less than about 7.2 mg/ml, less than about 7.1 mg/ml, less than about 7 mg/ml, less than about 6.9 mg/ml, less than about 6.8 mg/ml, less than about 6.7 mg/ml, less than about 6.6 mg/ml, less than about 6.5 mg/ml, less than about 6.4 mg/ml, less than about 6.3 mg/ml, less than about 6.2 mg/ml, less than about 6.1 mg/ml, less than about 6 mg/ml, less than about 5.9 mg/ml, less than about 5.8 mg/ml, less than about 5.7 mg/ml, less than about 5.6 mg/ml, less than about 5.5 mg/ml, less than about 5.4 mg/ml, less than about 5.3 mg/ml, less than about 5.2 mg/ml, less than about 5.1 mg/ml, less than about 5 mg/ml, less than about 4.9 mg/ml, less than about 4.8 mg/ml, less than about 4.7 mg/ml, less than about 4.6 mg/ml, less than about 4.5 mg/ml, less than about 4.4 mg/ml, less than about 4.3 mg/ml, less than about 4.2 mg/ml, less than about 4.1 mg/ml, less than about 4 mg/ml, less than about 3.9 mg/ml, less than about 3.8 mg/ml, less than about 3.7 mg/ml, less than about 3.6 mg/ml, less than about 3.5 mg/ml, less than about 3.4 mg/ml, less than about 3.3 mg/ml, less than about 3.2 mg/ml, less than about 3.1 mg/ml, less than about 3 mg/ml, less than about 2.9 mg/ml, less than about 2.8 mg/ml, less than about 2.7 mg/ml, less than about 2.6 mg/ml, less than about 2.5 mg/ml, less than about 2.4 mg/ml, less than about 2.3 mg/ml, less than about 2.2 mg/ml, less than about 2.1 mg/ml, less than about 2 mg/ml, less than about 1.9 mg/ml, less than about 1.8 mg/ml, less than about 1.7 mg/ml, less than about 1.6 mg/ml, less than about 1.5 mg/ml, less than about 1.4 mg/ml, less than about 1.3 mg/ml, less than about 1.2 mg/ml, less than about 1.1 mg/ml, or less than about 1 mg/ml, and chlorpheniramine, or a pharmaceutically acceptable salt, ester, hydrate, or solvate thereof, in an amount that is less than about 100 mg/ml, less than about 95 mg/ml, less than about 90 mg/ml, less than about 85 mg/ml, less than about 80 mg/ml, less than about 75 mg/ml, less than about 70 mg/ml, less than about 65 mg/ml, less than about 60 mg/ml, less than about 55 mg/ml, less than about 50 mg/ml, less than about 45 mg/ml, less than about 40 mg/ml, less than about 35 mg/ml, less than about 30 mg/ml, less than about 25 mg/ml, less than about 20 mg/ml, less than about 15 mg/ml, less than about 10 mg/ml less than about 9 mg/ml, less than about 8 mg/ml, less than about 7 mg/ml, less than about 6 mg/ml, less than about 5 mg/ml, less than about 4 mg/ml, less than about 3 mg/ml, less than about 2 mg/ml, less than about 1 mg/ml, less than about 0.9 mg/ml, less than about 0.8 mg/ml, less than about 0.7 mg/ml, less than about 0.6 mg/ml, less than about 0.5 mg/ml, less than about 0.4 mg/ml, less than about 0.3 mg/ml, or less than about 0.2 mg/ml.

In some embodiments, the aqueous-based composition contains phenylephrine, or a pharmaceutically acceptable salt, ester, hydrate, or solvate thereof, in an amount that is greater than about 1 mg/ml, greater than about 1.1 mg/ml, greater than about 1.2 mg/ml, greater than about 1.3 mg/ml, greater than about 1.4 mg/ml, greater than about 1.5 mg/ml, greater than about 1.6 mg/ml, greater than about 1.7 mg/ml, greater than about 1.8 mg/ml, greater than about 1.9 mg/ml, greater than about 2 mg/ml, greater than about 2.1 mg/ml, greater than about 2.2 mg/ml, greater than about 2.3 mg/ml, greater than about 2.4 mg/ml, greater than about 2.5 mg/ml, greater than about 2.6 mg/ml, greater than about 2.7 mg/ml, greater than about 2.8 mg/ml, greater than about 2.9 mg/ml, greater than about 3 mg/ml, greater than about 3.1 mg/ml, greater than about 3.2 mg/ml, greater than about 3.3 mg/ml, greater than about 3.4 mg/ml, greater than about 3.5 mg/ml, greater than about 3.6 mg/ml, greater than about 3.7 mg/ml, greater than about 3.8 mg/ml, greater than about 3.9 mg/ml, greater than about 4 mg/ml, greater than about 4.1 mg/ml, greater than about 4.2 mg/ml, greater than about 4.3 mg/ml, greater than about 4.4 mg/ml, greater than about 4.5 mg/ml, greater than about 4.6 mg/ml, greater than about 4.7 mg/ml, greater than about 4.8 mg/ml, greater than about 4.9 mg/ml, greater than about 5 mg/ml, greater than about 5.1 mg/ml, greater than about 5.2 mg/ml, greater than about 5.3 mg/ml, greater than about 5.4 mg/ml, greater than about 5.5 mg/ml, greater than about 5.6 mg/ml, greater than about 5.7 mg/ml, greater than about 5.8 mg/ml, greater than about 5.9 mg/ml, greater than about 6 mg/ml, greater than about 6.1 mg/ml, greater than about 6.2 mg/ml, greater than about 6.3 mg/ml, greater than about 6.4 mg/ml, greater than about 6.5 mg/ml, greater than about 6.6 mg/ml, greater than about 6.7 mg/ml, greater than about 6.8 mg/ml, greater than about 6.9 mg/ml, greater than about 7 mg/ml, greater than about 7.1 mg/ml, greater than about 7.2 mg/ml, greater than about 7.3 mg/ml, greater than about 7.4 mg/ml, greater than about 7.5 mg/ml, greater than about 7.6 mg/ml, greater than about 7.7 mg/ml w/v, greater than about 7.8 mg/ml, greater than about 7.9 mg/ml, greater than about 8 mg/ml, greater than about 8.1 mg/ml, greater than about 8.2 mg/ml, greater than about 8.3 mg/ml, greater than about 8.4 mg/ml, greater than about 8.5 mg/ml, greater than about 8.6 mg/ml, greater than about 8.7 mg/ml, greater than about 8.8 mg/ml, greater than about 8.9 mg/ml, greater than about 9 mg/ml, greater than about 9.1 mg/ml, greater than about 9.2 mg/ml, greater than about 9.3 mg/ml, greater than about 9.4 mg/ml, greater than about 9.5 mg/ml, greater than about 9.6 mg/ml, greater than about 9.7 mg/ml, greater than about 9.8 mg/ml, greater than about 9.9 mg/ml, or greater than about 10 mg/ml, and chlorpheniramine, or a pharmaceutically acceptable salt, ester, hydrate, or solvate thereof, in an amount that is greater than about 0.2 mg/ml, greater than about 0.3 mg/ml, greater than about 0.4 mg/ml, greater than about 0.5 mg/ml, greater than about 0.6 mg/ml, greater than about 0.7 mg/ml, greater than about 0.8 mg/ml, greater than about 0.9 mg/ml, greater than about 1 mg/ml, greater than about 2 mg/ml, greater than about 3 mg/ml, greater than about 4 mg/ml, greater than about 5 mg/ml, greater than about 6 mg/ml, greater than about 7 mg/ml, greater than about 8 mg/ml, greater than about 9 mg/ml, greater than about 10 mg/ml, greater than about 15 mg/ml, greater than about 20 mg/ml, greater than about 25 mg/ml, greater than about 30 mg/ml, greater than about 35 mg/ml, greater than about 40 mg/ml, greater than about 45 mg/ml, greater than about 50 mg/ml, greater than about 55 mg/ml, greater than about 60 mg/ml, greater than about 65 mg/ml, greater than about 70 mg/ml, greater than about 75 mg/ml, greater than about 80 mg/ml, greater than about 85 mg/ml, greater than about 90 mg/ml, greater than about 95 mg/ml, or greater than about 100 mg/ml.

In some embodiments, the aqueous-based composition contains phenylephrine, or a pharmaceutically acceptable salt, ester, hydrate, or solvate thereof, in an amount that has a range of mg/ml having an upper limit of about 10 mg/ml, 9 mg/ml, 8 mg/ml, 7 mg/ml, 6 mg/ml, 5 mg/ml, 4 mg/ml, 3 mg/ml, 2 mg/ml, or 1 mg/ml, and an independently selected lower limit of about 1 mg/ml, 2 mg/ml, 3 mg/ml, 4 mg/ml, 5 mg/ml, 6 mg/ml, 7 mg/ml, 8 mg/ml, 9 mg/ml, or 10 mg/ml, where the lower limit is less than the upper limit, and chlorpheniramine, or a pharmaceutically acceptable salt, ester, hydrate, or solvate thereof, in an amount that has a range of mg/ml having an upper limit of about 100 mg/ml, 80 mg/ml, 60 mg/ml, 40 mg/ml, 20 mg/ml, 10 mg/ml, 1 mg/ml, 0.8 mg/ml, 0.4 mg/ml, or 0.2 mg/ml and an independently selected lower limit of about 0.2 mg/ml, 0.4 mg/ml, 0.8 mg/ml, 1 mg/ml, 10 mg/ml, 20 mg/ml, 30 mg/ml, 40 mg/ml, 50 mg/ml, 60 mg/ml, 70 mg/ml, 80 mg/ml, 90 mg/ml, or 100 mg/ml, where the lower limit is less than the upper limit. In some embodiments, the aqueous-based composition contains phenylephrine, or a pharmaceutically acceptable salt, ester, hydrate, or solvate thereof, in an amount of about 1 mg/ml, about 2 mg/ml, about 3 mg/ml, about 4 mg/ml, about 5 mg/ml, about 6 mg/ml, about 7 mg/ml, about 8 mg/ml, about 9 mg/ml, about 10 mg/ml, and chlorpheniramine, or a pharmaceutically acceptable salt, ester, hydrate, or solvate thereof, in an amount of about 0.2 mg/ml, about 0.4 mg/ml, about 0.8 mg/ml, about 1 mg/ml, about 10 mg/ml, about 20 mg/ml, about 30 mg/ml, about 40 mg/ml, about 50 mg/ml, about 60 mg/ml, about 70 mg/ml, about 80 mg/ml, about 90 mg/ml, or about 100 mg/ml.

In some embodiments, the aqueous-based composition contains phenylephrine, or a pharmaceutically acceptable salt, ester, hydrate, or solvate thereof (e.g., phenylephrine HCl, etc.) in an amount that ranges from 0.01% w/v to about 0.05% w/v and chlorpheniramine, or a pharmaceutically acceptable salt, ester, hydrate, or solvate thereof (e.g., chlorpheniramine maleate, etc.) in an amount that ranges from 0.01% w/v to about 0.05% w/v. In some embodiments, the aqueous-based composition contains phenylephrine, or a pharmaceutically acceptable salt, ester, hydrate, or solvate thereof (e.g., phenylephrine HCl, etc.) in an amount of about 0.05% w/v, and chlorpheniramine, or a pharmaceutically acceptable salt, ester, hydrate, or solvate thereof (e.g., chlorpheniramine maleate, etc.) in an amount of about 0.02% w/v.

In some embodiments, the two APIs are diphenhydramine, or a pharmaceutically acceptable salt, ester, hydrate, or solvate thereof (e.g., diphenhydramine HCl, etc.), and phenylephrine, or a pharmaceutically acceptable salt, ester, hydrate, or solvate thereof (e.g., phenylephrine HCl, etc.). In some embodiments, the aqueous-based composition contains diphenhydramine, or a pharmaceutically acceptable salt, ester, hydrate, or solvate thereof in an amount that ranges from 2 mg/ml to 50 mg/ml and phenylephrine, or a pharmaceutically acceptable salt, ester, hydrate, or solvate thereof in an amount that ranges from 1 mg/ml to about 10 mg/ml. In some embodiments, the aqueous-based composition contains diphenhydramine, or a pharmaceutically acceptable salt, ester, hydrate, or solvate thereof, in an amount that is less than about 50 mg/ml, less than about 45 mg/ml, less than about 40 mg/ml, less than about 35 mg/ml, less than about 30 mg/ml, less than about 25 mg/ml, less than about 20 mg/ml, less than about 19 mg/ml, less than about 18 mg/ml, less than about 17 mg/ml, less than about 16 mg/ml, less than about 15 mg/ml, less than about 14 mg/ml, less than about 13 mg/ml, less than about 12 mg/ml, less than about 11 mg/ml, less than about 10 mg/ml, less than about 9.5 mg/ml, less than about 9 mg/ml, less than about 8.5 mg/ml, less than about 8 mg/ml, less than about 7.5 mg/ml, less than about 7 mg/ml, less than about 6.5 mg/ml, less than about 6 mg/ml, less than about 5.5 mg/ml, less than about 5 mg/ml, less than about 4.5 mg/ml, less than about 4 mg/ml, less than about 3.5 mg/ml, less than about 3 mg/ml, less than about 2.5 mg/ml, or less than about 2 mg/ml, and phenylephrine, or a pharmaceutically acceptable salt, ester, hydrate, or solvate thereof, in an amount that is less than about 10 mg/ml, less than about 9.9 mg/ml, less than about 9.8 mg/ml, less than about 9.7 mg/ml, less than about 9.6 mg/ml, less than about 9.5 mg/ml, less than about 9.4 mg/ml, less than about 9.3 mg/ml, less than about 9.2 mg/ml, less than about 9.1 mg/ml, less than about 9 mg/ml, less than about 8.9 mg/ml, less than about 8.8 mg/ml, less than about 8.7 mg/ml, less than about 8.6 mg/ml, less than about 8.5 mg/ml, less than about 8.4 mg/ml, less than about 8.3 mg/ml, less than about 8.2 mg/ml, less than about 8.1 mg/ml, less than about 8 mg/ml, less than about 7.9 mg/ml, less than about 7.8 mg/ml, less than about 7.7 mg/ml, less than about 7.6 mg/ml, less than about 7.5 mg/ml, less than about 7.4 mg/ml, less than about 7.3 mg/ml, less than about 7.2 mg/ml, less than about 7.1 mg/ml, less than about 7 mg/ml, less than about 6.9 mg/ml, less than about 6.8 mg/ml, less than about 6.7 mg/ml, less than about 6.6 mg/ml, less than about 6.5 mg/ml, less than about 6.4 mg/ml, less than about 6.3 mg/ml, less than about 6.2 mg/ml, less than about 6.1 mg/ml, less than about 6 mg/ml, less than about 5.9 mg/ml, less than about 5.8 mg/ml, less than about 5.7 mg/ml, less than about 5.6 mg/ml, less than about 5.5 mg/ml, less than about 5.4 mg/ml, less than about 5.3 mg/ml, less than about 5.2 mg/ml, less than about 5.1 mg/ml, less than about 5 mg/ml, less than about 4.9 mg/ml, less than about 4.8 mg/ml, less than about 4.7 mg/ml, less than about 4.6 mg/ml, less than about 4.5 mg/ml, less than about 4.4 mg/ml, less than about 4.3 mg/ml, less than about 4.2 mg/ml, less than about 4.1 mg/ml, less than about 4 mg/ml, less than about 3.9 mg/ml, less than about 3.8 mg/ml, less than about 3.7 mg/ml, less than about 3.6 mg/ml, less than about 3.5 mg/ml, less than about 3.4 mg/ml, less than about 3.3 mg/ml, less than about 3.2 mg/ml, less than about 3.1 mg/ml, less than about 3 mg/ml, less than about 2.9 mg/ml, less than about 2.8 mg/ml, less than about 2.7 mg/ml, less than about 2.6 mg/ml, less than about 2.5 mg/ml, less than about 2.4 mg/ml, less than about 2.3 mg/ml, less than about 2.2 mg/ml, less than about 2.1 mg/ml, less than about 2 mg/ml, less than about 1.9 mg/ml, less than about 1.8 mg/ml, less than about 1.7 mg/ml, less than about 1.6 mg/ml, less than about 1.5 mg/ml, less than about 1.4 mg/ml, less than about 1.3 mg/ml, less than about 1.2 mg/ml, less than about 1.1 mg/ml, or less than about 1 mg/ml.

In some embodiments, the aqueous-based composition contains diphenhydramine, or a pharmaceutically acceptable salt, ester, hydrate, or solvate thereof, in an amount that is greater than about 2 mg/ml, greater than about 2.5 mg/ml, greater than about 3 mg/ml, greater than about 3.5 mg/ml, greater than about 4 mg/ml, greater than about 4.5 mg/ml, greater than about 5 mg/ml, greater than about 5.5 mg/ml, greater than about 6 mg/ml, greater than about 6.5 mg/ml, greater than about 7 mg/ml, greater than about 7.5 mg/ml, greater than about 8 mg/ml, greater than about 8.5 mg/ml, greater than about 9 mg/ml, greater than about 9.5 mg/ml, greater than about 10 mg/ml, greater than about 11 mg/ml, greater than about 12 mg/ml, greater than about 13 mg/ml, greater than about 14 mg/ml, greater than about 15 mg/ml, greater than about 16 mg/ml, greater than about 17 mg/ml, greater than about 18 mg/ml, greater than about 19 mg/ml, greater than about 20 mg/ml, greater than about 25 mg/ml, greater than about 30 mg/ml, greater than about 35 mg/ml, greater than about 40 mg/ml, greater than about 45 mg/ml, or greater than about 50 mg/ml, and phenylephrine, or a pharmaceutically acceptable salt, ester, hydrate, or solvate thereof, in an amount that is greater than about 1 mg/ml, greater than about 1.1 mg/ml, greater than about 1.2 mg/ml, greater than about 1.3 mg/ml, greater than about 1.4 mg/ml, greater than about 1.5 mg/ml, greater than about 1.6 mg/ml, greater than about 1.7 mg/ml, greater than about 1.8 mg/ml, greater than about 1.9 mg/ml, greater than about 2 mg/ml, greater than about 2.1 mg/ml, greater than about 2.2 mg/ml, greater than about 2.3 mg/ml, greater than about 2.4 mg/ml, greater than about 2.5 mg/ml, greater than about 2.6 mg/ml, greater than about 2.7 mg/ml, greater than about 2.8 mg/ml, greater than about 2.9 mg/ml, greater than about 3 mg/ml, greater than about 3.1 mg/ml, greater than about 3.2 mg/ml, greater than about 3.3 mg/ml, greater than about 3.4 mg/ml, greater than about 3.5 mg/ml, greater than about 3.6 mg/ml, greater than about 3.7 mg/ml, greater than about 3.8 mg/ml, greater than about 3.9 mg/ml, greater than about 4 mg/ml, greater than about 4.1 mg/ml, greater than about 4.2 mg/ml, greater than about 4.3 mg/ml, greater than about 4.4 mg/ml, greater than about 4.5 mg/ml, greater than about 4.6 mg/ml, greater than about 4.7 mg/ml, greater than about 4.8 mg/ml, greater than about 4.9 mg/ml, greater than about 5 mg/ml, greater than about 5.1 mg/ml, greater than about 5.2 mg/ml, greater than about 5.3 mg/ml, greater than about 5.4 mg/ml, greater than about 5.5 mg/ml, greater than about 5.6 mg/ml, greater than about 5.7 mg/ml, greater than about 5.8 mg/ml, greater than about 5.9 mg/ml, greater than about 6 mg/ml, greater than about 6.1 mg/ml, greater than about 6.2 mg/ml, greater than about 6.3 mg/ml, greater than about 6.4 mg/ml, greater than about 6.5 mg/ml, greater than about 6.6 mg/ml, greater than about 6.7 mg/ml, greater than about 6.8 mg/ml, greater than about 6.9 mg/ml, greater than about 7 mg/ml, greater than about 7.1 mg/ml, greater than about 7.2 mg/ml, greater than about 7.3 mg/ml, greater than about 7.4 mg/ml, greater than about 7.5 mg/ml, greater than about 7.6 mg/ml, greater than about 7.7 mg/ml w/v, greater than about 7.8 mg/ml, greater than about 7.9 mg/ml, greater than about 8 mg/ml, greater than about 8.1 mg/ml, greater than about 8.2 mg/ml, greater than about 8.3 mg/ml, greater than about 8.4 mg/ml, greater than about 8.5 mg/ml, greater than about 8.6 mg/ml, greater than about 8.7 mg/ml, greater than about 8.8 mg/ml, greater than about 8.9 mg/ml, greater than about 9 mg/ml, greater than about 9.1 mg/ml, greater than about 9.2 mg/ml, greater than about 9.3 mg/ml, greater than about 9.4 mg/ml, greater than about 9.5 mg/ml, greater than about 9.6 mg/ml, greater than about 9.7 mg/ml, greater than about 9.8 mg/ml, greater than about 9.9 mg/ml, or greater than about 10 mg/ml.

In some embodiments, the aqueous-based composition contains diphenhydramine, or a pharmaceutically acceptable salt, ester, hydrate, or solvate thereof, in an amount that has a range of mg/ml having an upper limit of about 50 mg/ml, 40 mg/ml, 30 mg/ml, 20 mg/ml, 15 mg/ml, 10 mg/ml, 5 mg/ml, 2.5 mg/ml, or 2 mg/ml and an independently selected lower limit of about 2 mg/ml, 2.5 mg/ml, 5 mg/ml, 10 mg/ml, 15 mg/ml, 20 mg/ml, 30 mg/ml, 40 mg/ml, or 50 mg/ml, where the lower limit is less than the upper limit, and phenylephrine, or a pharmaceutically acceptable salt, ester, hydrate, or solvate thereof, in an amount that has a range of mg/ml having an upper limit of about 10 mg/ml, 9 mg/ml, 8 mg/ml, 7 mg/ml, 6 mg/ml, 5 mg/ml, 4 mg/ml, 3 mg/ml, 2 mg/ml, or 1 mg/ml, and an independently selected lower limit of about 1 mg/ml, 2 mg/ml, 3 mg/ml, 4 mg/ml, 5 mg/ml, 6 mg/ml, 7 mg/ml, 8 mg/ml, 9 mg/ml, or 10 mg/ml, where the lower limit is less than the upper limit. In some embodiments, the aqueous-based composition contains diphenhydramine, or a pharmaceutically acceptable salt, ester, hydrate, or solvate thereof, in an amount of about 2 mg/ml, about 2.5 mg/ml, about 5 mg/ml, about 10 mg/ml, about 15 mg/ml, about 20 mg/ml, about 30 mg/ml, about 40 mg/ml, or about 50 mg/ml, and phenylephrine, or a pharmaceutically acceptable salt, ester, hydrate, or solvate thereof, in an amount of about 1 mg/ml, about 2 mg/ml, about 3 mg/ml, about 4 mg/ml, about 5 mg/ml, about 6 mg/ml, about 7 mg/ml, about 8 mg/ml, about 9 mg/ml, or about 10 mg/ml.

Excipients

In some aspects, the present disclosure relates to aqueous-based compositions that contain any suitable known excipients. As used herein, an “excipient” refers to any non-active or inert ingredient used in a composition containing an active ingredient. Any suitable excipient may be used. Examples of suitable excipients include, without limitation, buffers, salts, sugars, stabilizers, diluents, antioxidants, solvents, chelators, thickeners, pH-adjusting agents, flavorants, colorants, sweeteners, and/or vehicles. In some embodiments, aqueous-based compositions of the present disclosure include one or more excipients that may be selected from pH-adjusting agents, flavorants, colorants, and sweeteners.

pH-Adjusting Agents

In some aspects, the present disclosure relates to aqueous-based compositions containing one or more (e.g., one or more, two or more, three or more, four or more, five or more, etc.) pH-adjusting agents. In some embodiments, the pH-adjusting agent may be any suitable acid, salts thereof, and derivatives thereof (e.g., acid anhydride derivatives, etc.). In some embodiments, the pH-adjusting agent is one or more of citric acid, fumaric acid, malic acid, phosphoric acid, succinic acid, tartaric acid, maleic acid, acetic acid, phosphoric acid, hydrochloric acid, lactic acid, propionic acid, salts thereof, and derivatives thereof (e.g., acid anhydride derivatives, etc.). In some embodiments, the pH-adjusting agent is one or more of sodium citrate, sodium phosphate monobasic, sodium phosphate dibasic, sodium acetate, ammonia, salts thereof, and derivatives thereof.

In some embodiments, the aqueous-based composition includes the pH-adjusting agent in an amount sufficient to yield a composition having a pH value that ranges from about 2.5 to about 8.

In some embodiments, an aqueous-based composition of the present disclosure has a pH value that ranges from about 2.5 to about 8. In some embodiments, the aqueous-based composition has a pH value that ranges from about 2.5 to about 8, from about 2.5 to about 7.5, from about 2.5 to about 7, from about 2.5 to about 6.5, from about 2.5 to about 6, from about 2.5 to about 5.5, from about 2.5 to about 5, from about 2.5 to about 4.5, from about 2.5 to about 4, from about 2.5 to about 3.5, from about 2.5 to about 3, from about 3 to about 8, from about 3 to about 7.5, from about 3 to about 7, from about 3 to about 6.5, from about 3 to about 6, from about 3 to about 5.5, from about 3 to about 5, from about 3 to about 4.5, from about 3 to about 4, from about 3 to about 3.5, from about 3.5 to about 8, from about 3.5 to about 7.5, from about 3.5 to about 7, from about 3.5 to about 6.5, from about 3.5 to about 6, from about 3.5 to about 5.5, from about 3.5 to about 5, from about 3.5 to about 4.5, from about 3.5 to about 4, from about 4 to about 8, from about 4 to about 7.5, from about 4 to about 7, from about 4 to about 6.5, from about 4 to about 6, from about 4 to about 5.5, from about 4 to about 5, from about 4 to about 4.5, from about 4.5 to about 8, from about 4.5 to about 7.5, from about 4.5 to about 7, from about 4.5 to about 6.5, from about 4.5 to about 6, from about 4.5 to about 5.5, from about 4.5 to about 5, from about 5 to about 8, from about 5 to about 7.5, from about 5 to about 7, from about 5 to about 6.5, from about 5 to about 6, from about 5 to about 5.5, from about 5.5 to about 8, from about 5.5 to about 7.5, from about 5.5 to about 7, from about 5.5 to about 6.5, from about 5.5 to about 6, from about 6 to about 8, from about 6 to about 7.5, from about 6 to about 7, from about 6 to about 6.5, from about 6.5 to about 8, from about 6.5 to about 7.5, from about 6.5 to about 7, from about 7 to about 8, from about 7 to about 7.5, or from about 7.5 to about 8. In some embodiments, the aqueous-based composition has a pH value of about 2.5, about 3, about 3.5, about 4, about 4.5, about 5, about 5.5, about 6, about 6.5, about 7, about 7.5, or about 8.

Flavorants

In some aspects, the present disclosure relates to aqueous-based compositions containing one or more (e.g., one or more, two or more, three or more, four or more, five or more, etc.) natural flavorants. As used herein, a “natural flavorant” refers to any flavorant that is an essential oil, oleoresin, essence or extractive, protein hydrolysate, distillate, or any product of roasting, heating or enzymolysis, which contains the flavoring constituents derived from a spice, fruit or fruit juice, vegetable or vegetable juice, edible yeast, herb, bark, bud, root, leaf or similar plant material, meat, fish, poultry, eggs, dairy products, or fermentation products thereof. Examples of such natural flavorants include, without limitation honey flavorants, grape flavorants, strawberry flavorants, raspberry flavorants, blue raspberry flavorants, tropical punch flavorants, and any combinations thereof.

In some embodiments, the present disclosure relates to aqueous-based compositions that do not contain any artificial flavorants. As used herein, an “artificial flavorant” refers to all flavorants that are not derived from a spice, fruit or fruit juice, vegetable or vegetable juice, edible yeast, herb, bark, bud, root, leaf or similar plant material, meat, fish, poultry, eggs, dairy products, or fermentation products thereof.

In some embodiments, the aqueous-based composition contains the one or more natural flavorants in an amount that ranges from about 0.05% w/v to about 1% w/v. In some embodiments, the aqueous-based composition contains one or more natural flavorants in an amount that is less than about 1% w/v, less than about 0.99% w/v, less than about 0.98% w/v, less than about 0.97% w/v, less than about 0.96% w/v, less than about 0.95% w/v, less than about 0.94% w/v, less than about 0.93% w/v, less than about 0.92% w/v, less than about 0.91% w/v, less than about 0.9% w/v, less than about 0.89% w/v, less than about 0.88% w/v, less than about 0.87% w/v, less than about 0.86% w/v, less than about 0.85% w/v, less than about 0.84% w/v, less than about 0.83% w/v, less than about 0.82% w/v, less than about 0.81% w/v, less than about 0.8% w/v, less than about 0.79% w/v, less than about 0.78% w/v, less than about 0.77% w/v, less than about 0.76% w/v, less than about 0.75% w/v, less than about 0.74% w/v, less than about 0.73% w/v, less than about 0.72% w/v, less than about 0.71% w/v, less than about 0.7% w/v, less than about 0.69% w/v, less than about 0.68% w/v, less than about 0.67% w/v, less than about 0.66% w/v, less than about 0.65% w/v, less than about 0.64% w/v, less than about 0.63% w/v, less than about 0.62% w/v, less than about 0.61% w/v, less than about 0.6% w/v, less than about 0.59% w/v, less than about 0.58% w/v, less than about 0.57% w/v, less than about 0.56% w/v, less than about 0.55% w/v, less than about 0.54% w/v, less than about 0.53% w/v, less than about 0.52% w/v, less than about 0.51% w/v, less than about 0.5% w/v, less than about 0.49% w/v, less than about 0.48% w/v, less than about 0.47% w/v, less than about 0.46% w/v, less than about 0.45% w/v, less than about 0.44% w/v, less than about 0.43% w/v, less than about 0.42% w/v, less than about 0.41% w/v, less than about 0.4% w/v, less than about 0.39% w/v, less than about 0.38% w/v, less than about 0.37% w/v, less than about 0.36% w/v, less than about 0.35% w/v, less than about 0.34% w/v, less than about 0.33% w/v, less than about 0.32% w/v, less than about 0.31% w/v, less than about 0.3% w/v, less than about 0.29% w/v, less than about 0.28% w/v, less than about 0.27% w/v, less than about 0.26% w/v, less than about 0.25% w/v, less than about 0.24% w/v, less than about 0.23% w/v, less than about 0.22% w/v, less than about 0.21% w/v, less than about 0.2% w/v, less than about 0.19% w/v, less than about 0.18% w/v, less than about 0.17% w/v, less than about 0.16% w/v, less than about 0.15% w/v, less than about 0.14% w/v, less than about 0.13% w/v, less than about 0.12% w/v, less than about 0.11% w/v, less than about 0.1% w/v, less than about 0.099% w/v, less than about 0.098% w/v, less than about 0.097% w/v, less than about 0.096% w/v, less than about 0.095% w/v, less than about 0.094% w/v, less than about 0.093% w/v, less than about 0.092% w/v, less than about 0.091% w/v, less than about 0.09% w/v, less than about 0.089% w/v, less than about 0.088% w/v, less than about 0.087% w/v, less than about 0.086% w/v, less than about 0.085% w/v, less than about 0.084% w/v, less than about 0.083% w/v, less than about 0.082% w/v, less than about 0.081% w/v, less than about 0.08% w/v, less than about 0.079% w/v, less than about 0.078% w/v, less than about 0.077% w/v, less than about 0.076% w/v, less than about 0.075% w/v, less than about 0.074% w/v, less than about 0.073% w/v, less than about 0.072% w/v, less than about 0.071% w/v, less than about 0.07% w/v, less than about 0.069% w/v, less than about 0.068% w/v, less than about 0.067% w/v, less than about 0.066% w/v, less than about 0.065% w/v, less than about 0.064% w/v, less than about 0.063% w/v, less than about 0.062% w/v, less than about 0.061% w/v, less than about 0.06% w/v, less than about 0.059% w/v, less than about 0.058% w/v, less than about 0.057% w/v, less than about 0.056% w/v, less than about 0.055% w/v, less than about 0.054% w/v, less than about 0.053% w/v, less than about 0.052% w/v, less than about 0.051% w/v, or less than about 0.05% w/v.

In some embodiments, the aqueous-based composition contains one or more natural flavorants in an amount that is greater than about 0.05% w/v, greater than about 0.051% w/v, greater than about 0.052% w/v, greater than about 0.053% w/v, greater than about 0.054% w/v, greater than about 0.055% w/v, greater than about 0.056% w/v, greater than about 0.057% w/v, greater than about 0.058% w/v, greater than about 0.059% w/v, greater than about 0.06% w/v, greater than about 0.061% w/v, greater than about 0.062% w/v, greater than about 0.063% w/v, greater than about 0.064% w/v, greater than about 0.065% w/v, greater than about 0.066% w/v, greater than about 0.067% w/v, greater than about 0.068% w/v, greater than about 0.069% w/v, greater than about 0.07% w/v, greater than about 0.071% w/v, greater than about 0.072% w/v, greater than about 0.073% w/v, greater than about 0.074% w/v, greater than about 0.075% w/v, greater than about 0.076% w/v, greater than about 0.077% w/v, greater than about 0.078% w/v, greater than about 0.079% w/v, greater than about 0.08% w/v, greater than about 0.081% w/v, greater than about 0.082% w/v, greater than about 0.083% w/v, greater than about 0.084% w/v, greater than about 0.085% w/v, greater than about 0.086% w/v, greater than about 0.087% w/v, greater than about 0.088% w/v, greater than about 0.089% w/v, greater than about 0.09% w/v, greater than about 0.091% w/v, greater than about 0.092% w/v, greater than about 0.093% w/v, greater than about 0.094% w/v, greater than about 0.095% w/v, greater than about 0.096% w/v, greater than about 0.097% w/v, greater than about 0.098% w/v, greater than about 0.099% w/v, greater than about 0.1% w/v, greater than about 0.11% w/v, greater than about 0.12% w/v, greater than about 0.13% w/v, greater than about 0.14% w/v, greater than about 0.15% w/v, greater than about 0.16% w/v, greater than about 0.17% w/v, greater than about 0.18% w/v, greater than about 0.19% w/v, greater than about 0.2% w/v, greater than about 0.21% w/v, greater than about 0.22% w/v, greater than about 0.23% w/v, greater than about 0.24% w/v, greater than about 0.25% w/v, greater than about 0.26% w/v, greater than about 0.27% w/v, greater than about 0.28% w/v, greater than about 0.29% w/v, greater than about 0.3% w/v, greater than about 0.31% w/v, greater than about 0.32% w/v, greater than about 0.33% w/v, greater than about 0.34% w/v, greater than about 0.35% w/v, greater than about 0.36% w/v, greater than about 0.37% w/v, greater than about 0.38% w/v, greater than about 0.39% w/v, greater than about 0.4% w/v, greater than about 0.41% w/v, greater than about 0.42% w/v, greater than about 0.43% w/v, greater than about 0.44% w/v, greater than about 0.45% w/v, greater than about 0.46% w/v, greater than about 0.47% w/v, greater than about 0.48% w/v, greater than about 0.49% w/v, greater than about 0.5% w/v, greater than about 0.51% w/v, greater than about 0.52% w/v, greater than about 0.53% w/v, greater than about 0.54% w/v, greater than about 0.55% w/v, greater than about 0.56% w/v, greater than about 0.57% w/v, greater than about 0.58% w/v, greater than about 0.59% w/v, greater than about 0.6% w/v, greater than about 0.61% w/v, greater than about 0.62% w/v, greater than about 0.63% w/v, greater than about 0.64% w/v, greater than about 0.65% w/v, greater than about 0.66% w/v, greater than about 0.67% w/v, greater than about 0.68% w/v, greater than about 0.69% w/v, greater than about 0.7% w/v, greater than about 0.71% w/v, greater than about 0.72% w/v, greater than about 0.73% w/v, greater than about 0.74% w/v, greater than about 0.75% w/v, greater than about 0.76% w/v, greater than about 0.77% w/v, greater than about 0.78% w/v, greater than about 0.79% w/v, greater than about 0.8% w/v, greater than about 0.81% w/v, greater than about 0.82% w/v, greater than about 0.83% w/v, greater than about 0.84% w/v, greater than about 0.85% w/v, greater than about 0.86% w/v, greater than about 0.87% w/v, greater than about 0.88% w/v, greater than about 0.89% w/v, greater than about 0.9% w/v, greater than about 0.91% w/v, greater than about 0.92% w/v, greater than about 0.93% w/v, greater than about 0.94% w/v, greater than about 0.95% w/v, greater than about 0.96% w/v, greater than about 0.97% w/v, greater than about 0.98% w/v, greater than about 0.99% w/v, or greater than about 1% w/v.

In some embodiments, the aqueous-based composition contains one or more natural flavorants in an amount that has a range of % w/v having an upper limit of about 1% w/v, 0.9%, 0.75% w/v, 0.5% w/v, 0.25% w/v, 0.1% w/v, 0.09% w/v, 0.075% w/v, 0.06% w/v, or 0.05% w/v, and an independently selected lower limit of about 0.05% w/v, 0.06% w/v, 0.075% w/v, 0.09% w/v, 0.1% w/v, 0.25% w/v, 0.5% w/v, 0.75% w/v, 0.9% w/v, or 1% w/v, where the lower limit is less than the upper limit. In some embodiments, the aqueous-based composition contains one or more APIs in an amount of about 0.05% w/v, about 0.06% w/v, about 0.075% w/v, about 0.09% w/v, about 0.1% w/v, about 0.25% w/v, about 0.5% w/v, about 0.75% w/v, about 0.9% w/v, or about 1% w/v.

Colorants

In some aspects, the present disclosure relates to aqueous-based compositions containing one or more (e.g., one or more, two or more, three or more, four or more, five or more, etc.) natural colorants. As used herein, a “natural colorant” refers to any colorant derived from a spice, fruit or fruit juice, vegetable or vegetable juice, edible yeast, herb, bark, bud, root, leaf or similar plant material, meat, fish, poultry, eggs, dairy products, or fermentation products thereof. Examples of such natural colorants may include, without limitation, caramel/malt, annatto, carmine, carrot oil, cottonseed flour, carotenoids (e.g., beta-carotene, lycopene, canthaxanthin, etc.), beet powder, beetroot red, cochineal/carmine, lutein, anthocyanin, grape skin extract, turmeric, mica-based pearlescent pigment, paprika (e.g., paprika oleoresin, etc.), riboflavin, saffron, spirulina, fruit juice (e.g., grape juice, etc.), vegetable juice (e.g., carrot juice, beet juice, etc.), and any combinations thereof. In some embodiments, aqueous-based compositions of the present disclosure contain one or more natural colorants selected from lutein, paprika, cochineal/carmine, beetroot red, anthocyanin, caramel/malt, and any combination thereof.

In some embodiments, the present disclosure relates to aqueous-based compositions that do not contain any artificial colorants. As used herein, the term “artificial colorant” refers to any colorant not derived from a spice, fruit or fruit juice, vegetable or vegetable juice, edible yeast, herb, bark, bud, root, leaf or similar plant material, meat, fish, poultry, eggs, dairy products, or fermentation products thereof. Examples of such artificial colorants may include, without limitation, D&C Red 40, D&C Red 3, FD&C Yellows 5 and 6, D&C Yellow 10, FD&C Blues 1 and 2, FD&C Green 3, and any combinations thereof.

In some embodiments, the aqueous-based composition contains the one or more natural colorants in an amount that ranges from about 0.01% w/v to about 0.05% w/v. In some embodiments, the aqueous-based composition contains the one or more natural colorants in an amount that is less than about 0.05% w/v, less than about 0.049% w/v, less than about 0.048% w/v, less than about 0.047% w/v, less than about 0.046% w/v, less than about 0.045% w/v, less than about 0.044% w/v, less than about 0.043% w/v, less than about 0.042% w/v, less than about 0.041% w/v, less than about 0.04% w/v, less than about 0.039% w/v, less than about 0.038% w/v, less than about 0.037% w/v, less than about 0.036% w/v, less than about 0.035% w/v, less than about 0.034% w/v, less than about 0.033% w/v, less than about 0.032% w/v, less than about 0.031% w/v, less than about 0.03% w/v, less than about 0.029% w/v, less than about 0.028% w/v, less than about 0.027% w/v, less than about 0.026% w/v, less than about 0.025% w/v, less than about 0.024% w/v, less than about 0.023% w/v, less than about 0.022% w/v, less than about 0.021% w/v, less than about 0.02% w/v, less than about 0.019% w/v, less than about 0.018% w/v, less than about 0.017% w/v, less than about 0.016% w/v, less than about 0.015% w/v, less than about 0.014% w/v, less than about 0.013% w/v, less than about 0.012% w/v, less than about 0.011% w/v, or less than about 0.01% w/v.

In some embodiments, the aqueous-based composition contains the one or more natural colorants in an amount that is greater than about 0.01% w/v, greater than about 0.011% w/v, greater than about 0.012% w/v, greater than about 0.013% w/v, greater than about 0.014% w/v, greater than about 0.015% w/v, greater than about 0.016% w/v, greater than about 0.017% w/v, greater than about 0.018% w/v, greater than about 0.019% w/v, greater than about 0.02% w/v, greater than about 0.021% w/v, greater than about 0.022% w/v, greater than about 0.023% w/v, greater than about 0.024% w/v, greater than about 0.025% w/v, greater than about 0.026% w/v, greater than about 0.027% w/v, greater than about 0.028% w/v, greater than about 0.029% w/v, greater than about 0.03% w/v, greater than about 0.031% w/v, greater than about 0.032% w/v, greater than about 0.033% w/v, greater than about 0.034% w/v, greater than about 0.035% w/v, greater than about 0.036% w/v, greater than about 0.037% w/v, greater than about 0.038% w/v, greater than about 0.039% w/v, greater than about 0.04% w/v, greater than about 0.041% w/v, greater than about 0.042% w/v, greater than about 0.043% w/v, greater than about 0.044% w/v, greater than about 0.045% w/v, greater than about 0.046% w/v, greater than about 0.047% w/v, greater than about 0.048% w/v, greater than about 0.049% w/v, or greater than about 0.05% w/v.

In some embodiments, the aqueous-based composition contains the one or more natural colorants in an amount that has a range of % w/v having an upper limit of about 0.05% w/v, 0.04% w/v, 0.03% w/v, 0.02% w/v, or 0.01% w/v, and an independently selected lower limit of about 0.01% w/v, 0.02% w/v, 0.03% w/v, 0.04% w/v, or 0.05% w/v, where the lower limit is less than the upper limit. In some embodiments, the aqueous-based composition contains the one or more natural colorants in an amount of about 0.01% w/v, about 0.02% w/v, about 0.03% w/v, about 0.04% w/v, or about 0.05% w/v.

Sweeteners

In some aspects, the present disclosure relates to aqueous-based compositions containing one or more (e.g., one or more, two or more, three or more, four or more, five or more, etc.) natural sweeteners. As used herein, a “natural sweetener” refers to any sweetener that is produced as a process of nature or results from processing a plant or any part thereof, and often contains sugar (sucrose) or sugar alcohols. The one or more sweeteners may be any suitable natural sweetener. Examples of suitable natural sweeteners include, without limitation, honey, differently processed sugars (e.g., refined, unrefined, brown, etc.), sugar cane juice, sugars from different plant sources (e.g., sugar cane, sugar beet, coconut, date, palm, maple, etc.), agave nectar, maple syrup, brown rice syrup, barley malt syrup, molasses, treacle, stevia, fruit juice concentrate, monk fruit extract, thaumatin, sugar alcohols, lactose, and/or vegetable glycerin.

In some embodiments, the present disclosure relates to aqueous-based compositions that do not contain any artificial sweeteners. As used herein, an “artificial sweetener” refers to any sweetener that is produced using specialized processes and requiring human agency, and often does not contain sugar (sucrose) or sugar alcohols. Examples of artificial sweeteners may include, without limitation, saccharin, aspartame, acesulfame potassium (acesulfame K), sucralose, neotame, cyclamates, neohesperidine, and advantame.

In some embodiments, the aqueous-based composition contains the one or more natural sweeteners in an amount that ranges from 50% w/v to about 70% w/v. In some embodiments, the aqueous-based composition contains the one or more natural sweeteners in an amount that is less than about 70% w/v, less than about 69.5% w/v, less than about 69% w/v, less than about 68.5% w/v, less than about 68% w/v, less than about 67.5% w/v, less than about 67% w/v, less than about 66.5% w/v, less than about 66% w/v, less than about 65.5% w/v, less than about 65% w/v, less than about 64.5% w/v, less than about 64% w/v, less than about 63.5% w/v, less than about 63% w/v, less than about 62.5% w/v, less than about 62% w/v, less than about 61.5% w/v, less than about 61% w/v, less than about 60.5% w/v, less than about 60% w/v, less than about 59.5% w/v, less than about 59% w/v, less than about 58.5% w/v, less than about 58% w/v, less than about 57.5% w/v, less than about 57% w/v, less than about 56.5% w/v, less than about 56% w/v, less than about 55.5% w/v, less than about 55% w/v, less than about 54.5% w/v, less than about 54% w/v, less than about 53.5% w/v, less than about 53% w/v, less than about 52.5% w/v, less than about 52% w/v, less than about 51.5% w/v, less than about 51% w/v, less than about 50.5% w/v, or less than about 50% w/v. In some embodiments, the aqueous-based composition contains the one or more natural sweeteners in an amount that is greater than about 50% w/v, greater than about 50.5% w/v, greater than about 51% w/v, greater than about 51.5% w/v, greater than about 52% w/v, greater than about 52.5% w/v, greater than about 53% w/v, greater than about 53.5% w/v, greater than about 54% w/v, greater than about 54.5% w/v, greater than about 55% w/v, greater than about 55.5% w/v, greater than about 56% w/v, greater than about 56.5% w/v, greater than about 57% w/v, greater than about 57.5% w/v, greater than about 58% w/v, greater than about 58.5% w/v, greater than about 59% w/v, greater than about 59.5% w/v, greater than about 60% w/v, greater than about 60.5% w/v, greater than about 61% w/v, greater than about 61.5% w/v, greater than about 62% w/v, greater than about 62.5% w/v, greater than about 63% w/v, greater than about 63.5% w/v, greater than about 64% w/v, greater than about 64.5% w/v, greater than about 65% w/v, greater than about 65.5% w/v, greater than about 66% w/v, greater than about 66.5% w/v, greater than about 67% w/v, greater than about 67.5% w/v, greater than about 68% w/v, greater than about 68.5% w/v, greater than about 69% w/v, greater than about 69.5% w/v, or greater than about 70% w/v. In some embodiments, the aqueous-based composition contains the one or more natural sweeteners in an amount that has a range of % w/v having an upper limit of about 70% w/v, 67.5% w/v, 65% w/v, 62.5% w/v, 60% w/v, 57.5% w/v, 55% w/v, 52.5% w/v, 50% w/v, and an independently selected lower limit of about 50% w/v, 52.5% w/v, 55% w/v, 57.5% w/v, 60% w/v, 62.5% w/v, 65% w/v, 67.5% w/v, or 70% w/v, where the lower limit is less than the upper limit. In some embodiments, the aqueous-based composition contains the one or more natural sweeteners in an amount of about 50% w/v, about 52.5% w/v, about 55% w/v, about 57.5% w/v, about 60% w/v, about 62.5% w/v, about 65% w/v, about 67.5% w/v, or about 70%. In some embodiments, the aqueous-based composition contains the one or more natural sweeteners in an amount that that ranges from about 10% w/v to about 50% w/v. In some embodiments, the aqueous-based composition contains the one or more natural sweeteners in an amount of about 10% w/v, about 15% w/v, about 25% w/v, about 35% w/v, about 45% w/v, or about 50% w/v.

Routes of Administration

In some aspects, the present disclosure relates to aqueous-based compositions that may be delivered via any suitable route of administration. Suitable routes of administration may include, without limitation, oral routes of administration, buccal routes of administration, sublingual routes of administration, inhalation routes of administration, nebulization routes of administration, ophthalmic routes of administration, otic routes of administration, nasal routes of administration, intravenous routes of administration, intramuscular routes of administration, intrathecal routes of administration, intraosseous routes of administration, rectal routes of administration, subcutaneous routes of administration, cutaneous routes of administration, and/or transdermal routes of administration. Accordingly, in some embodiments, an aqueous-based composition of the present disclosure is an oral composition that is administered via an oral route. In some embodiments, an aqueous-based composition of the present disclosure is a buccal composition that is administered via a buccal route. In some embodiments, an aqueous-based composition of the present disclosure is a sublingual composition that is administered via a sublingual route. In some embodiments, an aqueous-based composition of the present disclosure is an inhalation composition that is administered via an inhalation route. In some embodiments, an aqueous-based composition of the present disclosure is a nebulization composition that is administered via a nebulization route. In some embodiments, an aqueous-based composition of the present disclosure is an ophthalmic composition that is administered via an ophthalmic route. In some embodiments, an aqueous-based composition of the present disclosure is an otic composition that is administered via an otic route. In some embodiments, an aqueous-based composition of the present disclosure is a nasal composition that is administered via a nasal route. In some embodiments, an aqueous-based composition of the present disclosure is an intravenous composition that is administered via an intravenous route. In some embodiments, an aqueous-based composition of the present disclosure is an intramuscular composition that is administered via an intramuscular route. In some embodiments, an aqueous-based composition of the present disclosure is an intrathecal composition that is administered via an intrathecal route. In some embodiments, an aqueous-based composition of the present disclosure is an intraosseous composition that is administered via an intraosseous route. In some embodiments, an aqueous-based composition of the present disclosure is a rectal composition that is administered via a rectal route. In some embodiments, an aqueous-based composition of the present disclosure is a subcutaneous composition that is administered via a subcutaneous route. In some embodiments, an aqueous-based composition of the present disclosure is a cutaneous composition that is administered via a cutaneous route. In some embodiments, an aqueous-based composition of the present disclosure is a transdermal composition that is administered via a transdermal route.

Multi-Dosage Forms

In some aspects, the present disclosure relates to aqueous-based compositions in a multi-dosage form. As used herein, the term “multi-dosage” refers to an aqueous-based composition stored in a sealable container from which individual portions or doses may be taken. The advantages of the aqueous-based compositions of the present disclosure, which have antimicrobial effectiveness without containing preservatives, allow repeated dispensing of individual portions or doses without the risk of microbial contamination.

Multi-dosage forms of the present disclosure may be stored in any suitable container. A suitable container may include, without limitation, a sealable non-deformable container or a sealable deformable container. In some embodiments, the sealable non-deformable container may be a bottle, a cartridge, a syringe, and/or an inhaler. In some embodiments, the sealable non-deformable container may be used in conjunction with flexible tubing, an adjustable spigot, a hollow tip, a needle, or any other portion or dose delivery attachment known in the art. In some embodiments, the deformable container may be a bottle, a bag, a pouch, a tube, and/or a sack. In some embodiments, the sealable deformable container may be used in conjunction with flexible tubing, an adjustable spigot, a hollow tip, a needle, or any other portion or dose delivery attachment known in the art. In some embodiments, the suitable container may include means to reduce the risk of microbial growth and contamination associated with use. These means may include, without limitation, mechanical seals, self-sealing apertures, filtering systems, 0.2 micron or smaller filters, the use of negative pressure, and/or silver lining.

In some embodiments, the multi-dosage form may be a pharmaceutical composition stored in a sealable non-deformable or deformable container, and individual portions may be dispensed according to standards provided on the container. In some embodiments, the multi-dosage form may be a pharmaceutical composition stored in a sealable bottle and individual doses may be administered orally by dispensing onto a measuring spoon. In some embodiments, the multi-dosage form may be a pharmaceutical composition stored in a sealable inhaler and individual doses may be administered by dispensing a selected amount using the inhaler. In some embodiments, the multi-dosage form may be a pharmaceutical composition stored in a sealable syringe, individual doses may be dispensed using markings on the syringe, and the doses may be administered using a needle or a hollow tube. In some embodiments, the multi-dosage form may be a pharmaceutical composition stored in a sealable high-pressure syringe, individual doses may be dispensed using spacers, and the doses may be administered in aerosol form using a hollow tip. In some embodiments, the multi-dosage form may be a pharmaceutical composition stored in a sealable deformable bottle, and individual doses may be dispensed in drop form out of a hollow tip by squeezing the bottle. In some embodiments, the multi-dosage form may be a pharmaceutical composition stored in a sealable bag or pouch, and individual doses may be administered via intravenous therapy known in the art. In some embodiments, the multi-dosage form may be a nutritional composition stored in a sealable non-deformable or deformable container, and individual portions may be dispensed according to standards provided on the container or according to the user's preference. In some embodiments, the nutritional composition may be a beverage stored in a sealable non-deformable bottle, and individual portions may be dispensed by adjusting the position of the bottle to pour the beverage out or by using a mouthpiece. In some embodiments, the nutritional composition may be a beverage stored in a sealable deformable bottle, and individual portions may be dispensed by squeezing the bottle or by using a mouthpiece. In some embodiments, the multi-dosage form may be a nutritional composition stored in a sealable bag or pouch, and individual doses may be administered via intravenous therapy practices known in the art. In some embodiments, the multi-dosage form may be a nutritional composition stored in a sealable bag or pouch, and individual doses may be administered via tube feeding practices known in the art. In some embodiments, the multi-dosage form may be a cosmetic composition stored in a sealable non-deformable or deformable container, and individual portions may be dispensed according to standards provided on the container or according to the user's preference.

Multi-dosage forms of the present disclosure may contain one or more natural polymers, two or more sugar alcohols, one or more excipients, one or more thickening agents, one or more pH-adjusting agents, one or more natural sweeteners, one or more natural flavorants, one or more natural colorants, and/or water.

Formulated Aqueous-Based Compositions

In some embodiments, the aqueous-based composition containing a pharmaceutically effective amount of phenylephrine and dextromethorphan, the natural polymer xanthan gum, the natural polyols glycerin and sorbitol, and not containing any pharmaceutical antimicrobials or any artificial antimicrobial preservatives (e.g., butylparaben, methylparaben, propylparaben, EDTA, sodium benzoate, sodium propionate, or any combination thereof) contains the natural polymer xanthan gum in an amount that ranges from 0.5% w/v to 1% w/v, contains the natural polyol glycerin in an amount that ranges from about 50% w/v to about 70% w/v, and contains the sugar alcohol sorbitol in an amount that ranges from about 10% w/v to about 50% w/v. In some embodiments, the aqueous-based composition contains phenylephrine, or a pharmaceutically acceptable salt, ester, hydrate, or solvate thereof (e.g., phenylephrine HCl, phenylephrine bitartrate, etc.) in an amount that ranges from 0.01% w/v to 0.2% w/v and contains dextromethorphan, or a pharmaceutically acceptable salt, ester, hydrate, or solvate thereof (e.g., dextromethorphan HBr, etc.) in an amount that ranges from 0.01% w/v to 0.2% w/v. In some embodiments, the aqueous-based composition contains phenylephrine, or a pharmaceutically acceptable salt, ester, hydrate, or solvate thereof (e.g., phenylephrine HCl, phenylephrine bitartrate, etc.) in an amount that ranges from 0.01% w/v to 0.05% w/v and contains dextromethorphan, or a pharmaceutically acceptable salt, ester, hydrate, or solvate thereof (e.g., dextromethorphan HBr, etc.) in an amount that ranges from 0.01% w/v to 0.10% w/v. In some embodiments, the aqueous-based composition contains phenylephrine, or a pharmaceutically acceptable salt, ester, hydrate, or solvate thereof (e.g., phenylephrine HCl, phenylephrine bitartrate, etc.) in an amount of about 0.05% w/v and contains dextromethorphan, or a pharmaceutically acceptable salt, ester, hydrate, or solvate thereof (e.g., dextromethorphan HBr, etc.) in an amount of about 0.10% w/v. In some embodiments, the aqueous-based composition contains phenylephrine, or a pharmaceutically acceptable salt, ester, hydrate, or solvate thereof (e.g., phenylephrine HCl, phenylephrine bitartrate, etc.) in an amount that ranges from 0.3 mg/ml to 3 mg/ml and contains dextromethorphan, or a pharmaceutically acceptable salt, ester, hydrate, or solvate thereof (e.g., dextromethorphan HBr, etc.) in an amount that ranges from 1.3 mg/ml to 3 mg/ml. In some embodiments, the aqueous-based composition further includes citric acid, trisodium citrate, and water. In some embodiments, the citric acid is present in an amount of about 0.75% w/v. In some embodiments, the trisodium citrate is present in an amount of about 0.10% w/v.

In some embodiments, the aqueous-based composition containing a pharmaceutically effective amount of dextromethorphan and chlorpheniramine, the natural polymer xanthan gum, the natural polyols glycerin and sorbitol, and not containing any pharmaceutical antimicrobials or any artificial antimicrobial preservatives (e.g., butylparaben, methylparaben, propylparaben, EDTA, sodium benzoate, sodium propionate, or any combination thereof) contains the natural polymer xanthan gum in an amount that ranges from 0.5% w/v to 1% w/v, contains the natural polyol glycerin in an amount that ranges from about 50% w/v to about 70% w/v, and contains the sugar alcohol sorbitol in an amount that ranges from about 10% w/v to about 50% w/v. In some embodiments, the aqueous-based composition contains dextromethorphan, or a pharmaceutically acceptable salt, ester, hydrate, or solvate thereof (e.g., dextromethorphan HBr, etc.) in an amount that ranges from 0.01% w/v to 0.2% w/v and contains chlorpheniramine, or a pharmaceutically acceptable salt, ester, hydrate, or solvate thereof (e.g., chlorpheniramine maleate, etc.) in an amount that ranges from 0.01% w/v to 0.1% w/v. In some embodiments, the aqueous-based composition contains dextromethorphan, or a pharmaceutically acceptable salt, ester, hydrate, or solvate thereof (e.g., dextromethorphan HBr, etc.) in an amount of about 0.1% w/v and contains chlorpheniramine, or a pharmaceutically acceptable salt, ester, hydrate, or solvate thereof (e.g., chlorpheniramine maleate, etc.) in an amount of about 0.02% w/v. In some embodiments, the aqueous-based composition contains dextromethorphan, or a pharmaceutically acceptable salt, ester, hydrate, or solvate thereof (e.g., dextromethorphan HBr, etc.) in an amount that ranges from 1.3 mg/ml to 3 mg/ml and contains chlorpheniramine, or a pharmaceutically acceptable salt, ester, hydrate, or solvate thereof (e.g., chlorpheniramine maleate, etc.) in an amount that ranges from 0.2 mg/ml to 2 mg/ml. In some embodiments, the aqueous-based composition further includes citric acid, trisodium citrate, and water. In some embodiments, the citric acid is present in an amount of about 0.75% w/v. In some embodiments, the trisodium citrate is present in an amount of about 0.10% w/v.

In some embodiments, the aqueous-based composition containing a pharmaceutically effective amount of phenylephrine and chlorpheniramine, the natural polymer xanthan gum, the natural polyols glycerin and sorbitol, and not containing any pharmaceutical antimicrobials or any artificial antimicrobial preservatives (e.g., butylparaben, methylparaben, propylparaben, EDTA, sodium benzoate, sodium propionate, or any combination thereof) contains the natural polymer xanthan gum in an amount that ranges from 0.5% w/v to 1% w/v, contains the natural polyol glycerin in an amount that ranges from about 50% w/v to about 70% w/v, and contains the sugar alcohol sorbitol in an amount that ranges from about 10% w/v to about 50% w/v. In some embodiments, the aqueous-based composition contains phenylephrine, or a pharmaceutically acceptable salt, ester, hydrate, or solvate thereof (e.g., phenylephrine HCl, phenylephrine bitartrate, etc.) in an amount that ranges from 0.01% w/v to 0.2% w/v and contains chlorpheniramine, or a pharmaceutically acceptable salt, ester, hydrate, or solvate thereof (e.g., chlorpheniramine maleate, etc.) in an amount that ranges from 0.01% w/v to 0.1% w/v. In some embodiments, the aqueous-based composition contains phenylephrine, or a pharmaceutically acceptable salt, ester, hydrate, or solvate thereof (e.g., phenylephrine HCl, phenylephrine bitartrate, etc.) in an amount of about 0.05% w/v and contains chlorpheniramine, or a pharmaceutically acceptable salt, ester, hydrate, or solvate thereof (e.g., chlorpheniramine maleate, etc.) in an amount of about 0.02% w/v. In some embodiments, the aqueous-based composition contains phenylephrine, or a pharmaceutically acceptable salt, ester, hydrate, or solvate thereof (e.g., phenylephrine HCl, phenylephrine bitartrate, etc.) in an amount that ranges from 0.3 mg/ml to 3 mg/ml and contains chlorpheniramine, or a pharmaceutically acceptable salt, ester, hydrate, or solvate thereof (e.g., chlorpheniramine maleate, etc.) in an amount that ranges from 0.2 mg/ml to 2 mg/ml. In some embodiments, the aqueous-based composition further includes citric acid, trisodium citrate, and water. In some embodiments, the citric acid is present in an amount of about 0.75% w/v. In some embodiments, the trisodium citrate is present in an amount of about 0.10% w/v.

In some embodiments, the aqueous-based composition containing a pharmaceutically effective amount of diphenhydramine and phenylephrine, the natural polymer xanthan gum, the natural polyols glycerin and sorbitol, and not containing any pharmaceutical antimicrobials or any artificial antimicrobial preservatives (e.g., butylparaben, methylparaben, propylparaben, EDTA, sodium benzoate, sodium propionate, or any combination thereof) contains the natural polymer xanthan gum in an amount that ranges from 0.5% w/v to 1% w/v, contains the natural polyol glycerin in an amount that ranges from about 50% w/v to about 70% w/v, and contains the sugar alcohol sorbitol in an amount that ranges from about 10% w/v to about 50% w/v. In some embodiments, the aqueous-based composition contains diphenhydramine, or a pharmaceutically acceptable salt, ester, hydrate, or solvate thereof (e.g., diphenhydramine HCl, etc.) in an amount that ranges from 0.1% w/v to 0.8% w/v and contains phenylephrine, or a pharmaceutically acceptable salt, ester, hydrate, or solvate thereof (e.g., phenylephrine HCl, phenylephrine bitartrate, etc.) in an amount that ranges from 0.01% w/v to 0.2% w/v. In some embodiments, the aqueous-based composition contains diphenhydramine, or a pharmaceutically acceptable salt, ester, hydrate, or solvate thereof (e.g., diphenhydramine HCl, etc.) in an amount of about 0.25% w/v and contains phenylephrine, or a pharmaceutically acceptable salt, ester, hydrate, or solvate thereof (e.g., phenylephrine HCl, phenylephrine bitartrate, etc.) in an amount of about 0.05% w/v. In some embodiments, the aqueous-based composition contains diphenhydramine, or a pharmaceutically acceptable salt, ester, hydrate, or solvate thereof (e.g., diphenhydramine HCl, etc.) in an amount that ranges from 1.5 mg/ml to 7.5 mg/ml and contains phenylephrine, or a pharmaceutically acceptable salt, ester, hydrate, or solvate thereof (e.g., phenylephrine HCl, phenylephrine bitartrate, etc.) in an amount that ranges from 0.3 mg/ml to 3 mg/ml. In some embodiments, the aqueous-based composition further includes citric acid, trisodium citrate, and water. In some embodiments, the citric acid is present in an amount of about 0.75% w/v. In some embodiments, the trisodium citrate is present in an amount of about 0.10% w/v.

In some embodiments, the aqueous-based composition containing a pharmaceutically effective amount of phenylephrine and dextromethorphan, the natural polymer xanthan gum in an amount effective to promote microbial growth, and the natural polyols glycerin and sorbitol in an amount sufficient to inhibit the microbial growth promoted by the natural polymer xanthan gum contains the natural polymer xanthan gum in an amount that ranges from 0.5% w/v to 1% w/v, contains the natural polyol glycerin in an amount that ranges from about 50% w/v to about 70% w/v, and contains the sugar alcohol sorbitol in an amount that ranges from about 10% w/v to about 50% w/v. In some embodiments, the aqueous-based composition contains phenylephrine, or a pharmaceutically acceptable salt, ester, hydrate, or solvate thereof (e.g., phenylephrine HCl, phenylephrine bitartrate, etc.) in an amount that ranges from 0.01% w/v to 0.2% w/v and contains dextromethorphan, or a pharmaceutically acceptable salt, ester, hydrate, or solvate thereof (e.g., dextromethorphan HBr, etc.) in an amount that ranges from 0.01% w/v to 0.2% w/v. In some embodiments, the aqueous-based composition contains phenylephrine, or a pharmaceutically acceptable salt, ester, hydrate, or solvate thereof (e.g., phenylephrine HCl, phenylephrine bitartrate, etc.) in an amount that ranges from 0.01% w/v to 0.05% w/v and contains dextromethorphan, or a pharmaceutically acceptable salt, ester, hydrate, or solvate thereof (e.g., dextromethorphan HBr, etc.) in an amount that ranges from 0.01% w/v to 0.10% w/v. In some embodiments, the aqueous-based composition contains phenylephrine, or a pharmaceutically acceptable salt, ester, hydrate, or solvate thereof (e.g., phenylephrine HCl, phenylephrine bitartrate, etc.) in an amount of about 0.05% w/v and contains dextromethorphan, or a pharmaceutically acceptable salt, ester, hydrate, or solvate thereof (e.g., dextromethorphan HBr, etc.) in an amount of about 0.10% w/v. In some embodiments, the aqueous-based composition contains phenylephrine, or a pharmaceutically acceptable salt, ester, hydrate, or solvate thereof (e.g., phenylephrine HCl, phenylephrine bitartrate, etc.) in an amount that ranges from 0.3 mg/ml to 3 mg/ml and contains dextromethorphan, or a pharmaceutically acceptable salt, ester, hydrate, or solvate thereof (e.g., dextromethorphan HBr, etc.) in an amount that ranges from 1.3 mg/ml to 3 mg/ml. In some embodiments, the aqueous-based composition further includes citric acid, trisodium citrate, and water. In some embodiments, the citric acid is present in an amount of about 0.75% w/v. In some embodiments, the trisodium citrate is present in an amount of about 0.10% w/v.

In some embodiments, the aqueous-based composition containing a pharmaceutically effective amount of dextromethorphan and chlorpheniramine, the natural polymer xanthan gum in an amount effective to promote microbial growth, and the natural polyols glycerin and sorbitol in an amount sufficient to inhibit the microbial growth promoted by the natural polymer xanthan gum contains the natural polymer xanthan gum in an amount that ranges from 0.5% w/v to 1% w/v, contains the natural polyol glycerin in an amount that ranges from about 50% w/v to about 70% w/v, and contains the sugar alcohol sorbitol in an amount that ranges from about 10% w/v to about 50% w/v. In some embodiments, the aqueous-based composition contains dextromethorphan, or a pharmaceutically acceptable salt, ester, hydrate, or solvate thereof (e.g., dextromethorphan HBr, etc.) in an amount that ranges from 0.01% w/v to 0.2% w/v and contains chlorpheniramine, or a pharmaceutically acceptable salt, ester, hydrate, or solvate thereof (e.g., chlorpheniramine maleate, etc.) in an amount that ranges from 0.01% w/v to 0.1% w/v. In some embodiments, the aqueous-based composition contains dextromethorphan, or a pharmaceutically acceptable salt, ester, hydrate, or solvate thereof (e.g., dextromethorphan HBr, etc.) in an amount of about 0.1% w/v and contains chlorpheniramine, or a pharmaceutically acceptable salt, ester, hydrate, or solvate thereof (e.g., chlorpheniramine maleate, etc.) in an amount of about 0.02% w/v. In some embodiments, the aqueous-based composition contains dextromethorphan, or a pharmaceutically acceptable salt, ester, hydrate, or solvate thereof (e.g., dextromethorphan HBr, etc.) in an amount that ranges from 1.3 mg/ml to 3 mg/ml and contains chlorpheniramine, or a pharmaceutically acceptable salt, ester, hydrate, or solvate thereof (e.g., chlorpheniramine maleate, etc.) in an amount that ranges from 0.2 mg/ml to 2 mg/ml. In some embodiments, the aqueous-based composition further includes citric acid, trisodium citrate, and water. In some embodiments, the citric acid is present in an amount of about 0.75% w/v. In some embodiments, the trisodium citrate is present in an amount of about 0.10% w/v.

In some embodiments, the aqueous-based composition containing a pharmaceutically effective amount of phenylephrine and chlorpheniramine, the natural polymer xanthan gum in an amount effective to promote microbial growth, and the natural polyols glycerin and sorbitol in an amount sufficient to inhibit the microbial growth promoted by the natural polymer xanthan gum contains the natural polymer xanthan gum in an amount that ranges from 0.5% w/v to 1% w/v, contains the natural polyol glycerin in an amount that ranges from about 50% w/v to about 70% w/v, and contains the sugar alcohol sorbitol in an amount that ranges from about 10% w/v to about 50% w/v. In some embodiments, the aqueous-based composition contains phenylephrine, or a pharmaceutically acceptable salt, ester, hydrate, or solvate thereof (e.g., phenylephrine HCl, phenylephrine bitartrate, etc.) in an amount that ranges from 0.01% w/v to 0.2% w/v and contains chlorpheniramine, or a pharmaceutically acceptable salt, ester, hydrate, or solvate thereof (e.g., chlorpheniramine maleate, etc.) in an amount that ranges from 0.01% w/v to 0.1% w/v. In some embodiments, the aqueous-based composition contains phenylephrine, or a pharmaceutically acceptable salt, ester, hydrate, or solvate thereof (e.g., phenylephrine HCl, phenylephrine bitartrate, etc.) in an amount of about 0.05% w/v and contains chlorpheniramine, or a pharmaceutically acceptable salt, ester, hydrate, or solvate thereof (e.g., chlorpheniramine maleate, etc.) in an amount of about 0.02% w/v. In some embodiments, the aqueous-based composition contains phenylephrine, or a pharmaceutically acceptable salt, ester, hydrate, or solvate thereof (e.g., phenylephrine HCl, phenylephrine bitartrate, etc.) in an amount that ranges from 0.3 mg/ml to 3 mg/ml and contains chlorpheniramine, or a pharmaceutically acceptable salt, ester, hydrate, or solvate thereof (e.g., chlorpheniramine maleate, etc.) in an amount that ranges from 0.2 mg/ml to 2 mg/ml. In some embodiments, the aqueous-based composition further includes citric acid, trisodium citrate, and water. In some embodiments, the citric acid is present in an amount of about 0.75% w/v. In some embodiments, the trisodium citrate is present in an amount of about 0.10% w/v.

In some embodiments, the aqueous-based composition containing a pharmaceutically effective amount of diphenhydramine and phenylephrine, the natural polymer xanthan gum in an amount effective to promote microbial growth, and the natural polyols glycerin and sorbitol in an amount sufficient to inhibit the microbial growth promoted by the natural polymer xanthan gum contains the natural polymer xanthan gum in an amount that ranges from 0.5% w/v to 1% w/v, contains the natural polyol glycerin in an amount that ranges from about 50% w/v to about 70% w/v, and contains the sugar alcohol sorbitol in an amount that ranges from about 10% w/v to about 50% w/v. In some embodiments, the aqueous-based composition contains diphenhydramine, or a pharmaceutically acceptable salt, ester, hydrate, or solvate thereof (e.g., diphenhydramine HCl, etc.) in an amount that ranges from 0.1% w/v to 0.8% w/v and contains phenylephrine, or a pharmaceutically acceptable salt, ester, hydrate, or solvate thereof (e.g., phenylephrine HCl, phenylephrine bitartrate, etc.) in an amount that ranges from 0.01% w/v to 0.2% w/v. In some embodiments, the aqueous-based composition contains diphenhydramine, or a pharmaceutically acceptable salt, ester, hydrate, or solvate thereof (e.g., diphenhydramine HCl, etc.) in an amount of about 0.25% w/v and contains phenylephrine, or a pharmaceutically acceptable salt, ester, hydrate, or solvate thereof (e.g., phenylephrine HCl, phenylephrine bitartrate, etc.) in an amount of about 0.05% w/v. In some embodiments, the aqueous-based composition contains diphenhydramine, or a pharmaceutically acceptable salt, ester, hydrate, or solvate thereof (e.g., diphenhydramine HCl, etc.) in an amount that ranges from 1.5 mg/ml to 7.5 mg/ml and contains phenylephrine, or a pharmaceutically acceptable salt, ester, hydrate, or solvate thereof (e.g., phenylephrine HCl, phenylephrine bitartrate, etc.) in an amount that ranges from 0.3 mg/ml to 3 mg/ml. In some embodiments, the aqueous-based composition further includes citric acid, trisodium citrate, and water. In some embodiments, the citric acid is present in an amount of about 0.75% w/v. In some embodiments, the trisodium citrate is present in an amount of about 0.10% w/v.

### EXAMPLES

The present disclosure will be more fully understood by reference to the following examples. They should not, however, be construed as limiting the scope of the present disclosure. It is understood that the examples and embodiments described herein are for illustrative purposes only and that various modifications or changes in light thereof will be suggested to persons skilled in the art and are to be included within the spirit and purview of this application and scope of the appended claims.

### Example 1

### Effects of Different Parameters on Antimicrobial Effectiveness Testing

The following example describes the effects of excipient concentration, natural polymer concentration, pH, and viscosity on the antimicrobial effectiveness of aqueous-based compositions lacking an antimicrobial preservative and containing one or more active pharmaceutical ingredients.

Materials and Methods

Antimicrobial Effectiveness Testing: The antimicrobial effectiveness testing (AET) for aqueous-based compositions containing the sugar alcohols glycerin and sorbitol and the natural polymer xanthan gum was assessed according to USP <51> Antimicrobial Effectiveness Testing. Specifically, the Criteria for Antimicrobial Effectiveness for Category 3 products were used to determine antimicrobial effectiveness of the aqueous-based compositions. The organisms used were those listed in the USP <51> Criteria, and include Escherichia coli (ATCC 8739), Staphylococcus aureus (ATCC 6538), Pseudomonas aeruginosa (ATCC 9027), Candida albicans (ATCC 10231), and Aspergillus brasiliensis (ATCC 16404), which were cultured using the appropriate liquid medium for each strain and the conditions described in the Preparation of Test Strains section of the USP <51> Criteria.

The AET testing procedure is described in the Procedure section of the USP <51> Criteria. Briefly, each test composition was aliquoted into five sterile, capped bacteriological containers. Then, each container was inoculated with one of the prepared and standardized inocula containing the test organisms, and mixed. The concentration of test organisms added to the test composition was such that the concentration of the organisms after inoculation was between 1×10⁵ and 1×10⁶ cfu/mL of the test composition. This starting concentration was estimated based on the concentration of microorganisms in each of the standardized inocula as determined by the plate-count method (i.e., using a minimum of duplicate plates and then averaging the cfu before determining cfu/mL). Following inoculation, the inoculated containers were incubated at 22.5° C.±2.5°. Each container was sampled at 14 and 28 days, at which time any changes observed in appearance were recorded and the plate-count procedure was used to determine the number of cfu present in each test composition. Finally, the change in logio values of the concentration of cfu/mL for each microorganism was calculated. Changes in concentration were expressed in terms of log reductions, which were defined as the difference between the logio unit value of the starting concentration of cfu/mL in the suspension and the logio unit value of cfu/mL of the survivors at the time point of sampling.

According to the USP <51> criteria, compositions were AET compliant if said compositions were able to inhibit the growth of bacteria, yeast, and molds at 14 days and at 28 days. Bacterial growth was considered inhibited if at 14 days there was not less than a 1.0 logio reduction from the initial count, and no increase from the count at 14 days by 28 days. Yeast and mold growth was considered inhibited if there was no increase from the initial calculated count at 14 days and at 28 days. No increase was defined as not more than 0.5 logio unit higher than the previous value measured.

Results

To test the effects of different parameters on the antimicrobial effectiveness of aqueous-based compositions lacking an antimicrobial preservative and containing one or more active pharmaceutical ingredients, a series of aqueous-based compositions were developed. These compositions contained purified water, the buffer components, glycerin and sorbitol in varying amounts, and were developed to examine the effects of excipient concentration, natural polymer concentration, pH, and viscosity. The active ingredients Dextromethorphan HBr and Phenylephrine HBr were added to each composition (Table 1-Table 3). Composition 1613686-030 was identified as an aqueous-based composition lacking an artificial antimicrobial preservative that nonetheless had antimicrobial effectiveness, and was therefore used as a positive control in these assays.

Some of the test compositions were designed to examine the effects of excipient concentration. For example, 1613686-042 omitted glycerin entirely, while 1613686-043 omitted sorbitol entirely, and 1613686-041 omitted both glycerin and sorbitol (Table 1). Other compositions tested different glycerin concentrations, such as the composition 1613686-044 that contained 30% glycerin (Table 2). In all compositions, sorbitol was either present at 10% concentration or omitted entirely (Table 1 & Table 2).

TABLE 1
Aqueous-based compositions designed to test effect of excipient concentration

| Ingredients | 1613686-030 | 1613686-041 | 1613686-042 | 1613686-043 |
|---|---|---|---|---|
| Dextromethorphan HBr | 0.100% | 0.100% | 0.100% | 0.100% |
| Phenylephrine HCl | 0.050% | 0.050% | 0.050% | 0.050% |
| Citric acid, Anhydrous | 0.750% | 0.750% | 0.750% | 0.750% |
| Trisodium Citrate, dihydrate | 0.100% | 0.100% | 0.100% | 0.100% |
| Glycerin, USP | 50.000% | | | 50.000% |
| Sorbitol Solution USP | 10.000% | | 10.000% | |
| Xanthan gum (Xantural 180) | 0.250% | 0.250% | 0.250% | 0.250% |
| Purified water | q.s. | q.s. | q.s. | q.s. |
| pH | 3.02 | 2.78 | 2.77 | 2.86 |
| Viscosity (cP) at room | 290 | 149 | 160 | 224 |
| temperature (LV2 at 30 rpm) | | | | |
| AET complies (Yes/No) | Yes | No | No | No |

TABLE 2
Aqueous-based compositions designed to test effect
of excipient concentration, pH, and viscosity

| Ingredients | 1613686-044 | 1613686-045 | 1613686-046 | 1613686-047 |
|---|---|---|---|---|
| Dextromethorphan HBr | 0.100% | 0.100% | 0.100% | 0.100% |
| Phenylephrine HCl | 0.050% | 0.050% | 0.050% | 0.050% |
| Citric acid, Anhydrous | 0.750% | 0.750% | 0.0034% | 0.750% |
| Trisodium Citrate, dihydrate | 0.100% | 0.100% | 0.289% | 0.100% |
| Glycerin, USP | 30.000% | 10.000% | 50.000% | 40.000% |
| Sorbitol Solution USP | 10.000% | 10.000% | 10.000% | 10.000% |
| Xanthan gum (Xantural 180) | 0.250% | 0.250% | 0.250% | 0.250% |
| Purified water | q.s. | q.s. | q.s. | q.s. |
| pH | 2.75 | 2.71 | 7.60 | 2.87 |
| Viscosity (cP) at room | 245 | 363 | 203 | 280 |
| temperature (LV2 at 30 rpm) | | | | |
| AET complies (Yes/No) | No | No | Yes | No |

Some of the test compositions were designed to examine the effects of natural polymer concentration. In compositions where natural polymer concentrations were being tested, xanthan gum was present at 0.375%, 0.500%, or 0.750%. At each of these concentrations, the effect of increased xanthan gum concentration was examined either with 50% glycerin and 10% sorbitol or without both. For example, composition 1613686-048 contained 0.750% xanthan gum, 50.000% glycerin and 10.000% sorbitol, while composition 1613686-049 contained 0.750% xanthan gum (Table 3). In compositions where excipient concentrations were being tested, xanthan gum was present at 0.25% (Table 1 & Table 2).

TABLE 3
Aqueous-based compositions designed to test effect of natural polymer concentration

| Ingredients | 1613686-030 | 1613686-048 | 1613686-049 | 1613686-050 | 1613686-051 | 1613686-052 | 1613686-053 |
|---|---|---|---|---|---|---|---|
| Dextromethorphan | 0.100% | 0.100% | 0.100% | 0.100% | 0.100% | 0.100% | 0.100% |
| HBr | | | | | | | |
| Phenylephrine HCl | 0.050% | 0.050% | 0.050% | 0.050% | 0.050% | 0.050% | 0.050% |
| Citric acid, Anhydrous | 0.750% | 0.750% | 0.750% | 0.750% | 0.750% | 0.750% | 0.750% |
| Trisodium Citrate, | 0.100% | 0.100% | 0.100% | 0.100% | 0.100% | 0.100% | 0.100% |
| dihydrate | | | | | | | |
| Glycerin, USP | 50.000% | 50.000% | | 50.000% | | 50.000% | |
| Sorbitol Solution USP | 10.000% | 10.000% | | 10.000% | | 10.000% | |
| Xanthan gum | 0.250% | 0.750% | 0.750% | 0.500% | 0.500% | 0.375% | 0.375% |
| (Xantural 180) | | | | | | | |
| Purified water | q.s. | q.s. | q.s. | q.s. | q.s. | q.s. | q.s. |
| pH | 3.02 | 3.05 | 2.86 | 3.01 | 2.84 | 2.97 | 2.87 |
| Viscosity (cP) at | 290 | 1697 | 1280 | 892 | 640 | TBD | 332 |
| room temperature | | | | | | | |
| (LV2 at 30 rpm) | | | | | | | |
| AET complies | Yes | Yes | No | Yes | No | Yes | No |
| (Yes/No) | | | | | | | |

Some of the test compositions were designed to examine the effects of both pH and viscosity. A slightly alkaline pH of 7.6 was tested in composition 1613686-046 (Table 2), which was achieved by altering the concentrations of citric acid and trisodium citrate. The results from these compositions indicate the antimicrobial effectiveness is present in the pH range of 3 to 8. Furthermore, viscosities ranging from 149.3 to 1697 were tested (Table 1, Table 2 & Table 3). The range of viscosities was achieved by varying the xanthan gum concentration, which directly affected the viscosity of the final solution.

The aqueous-based compositions were evaluated using AET, as described above (Tables 4A-4F; compositions grouped on the basis of common inoculum concentration values). Almost all of the compositions inhibited the growth of all three bacterial species (E. coli, S. aureus, and P. aeruginosa) at day 14 and day 28, with the exception of composition 1613686-049, which had increased bacterial growth at day 14 and day 28 (indicated by bold values in Table 4D). The bacterial growth observed in composition 1613686-049 was not indicative of the microorganism species used in AET. This growth, however, was observed prior to initiating the AET study, which indicates that composition 1613686-049 provided a substrate for microbial growth (i.e., was not antimicrobially effective). Thus, the microbial growth seen in this composition during AET testing confirms that composition 1613686-049 does not satisfy the criteria of antimicrobial effectiveness. The composition 1613686-051 showed an increased count of the yeast species C. albicans at days 14 and 28, as did the composition 1613686-053 (indicated by bold values in Table 4F). The compositions 1613686-049, 1613686-051, and 1613686-053 lack glycerin and sorbitol. The AET results for these three compositions appear to indicate that xanthan gum alone is not sufficient to inhibit microbial growth, and that glycerin and sorbitol may be required for antimicrobial effectiveness. Moreover, these results indicate that higher amounts of xanthan gum, as in 1613686-049, perform worse in AET testing.

Three compositions, one example being composition 1613686-043, showed an increased count of the mold species A. brasiliensis (indicated by bold values in Tables 4C & 4E), but no increase in growth of the other microorganisms. Four compositions, for example the composition 1613686-042, showed an increased count of both mold and yeast species (indicated by bold values in Tables 4B, 4C & 4F; “TNTC” is an abbreviation for “too numerous to count”). Of these compositions, 1613686-043 was missing sorbitol, 1613686-042 was missing glycerin, and 1613686-041 was missing both glycerin and sorbitol. These results further indicate that both glycerin and sorbitol may be required for antimicrobial effectiveness. The results from compositions 1613686-044, 1613686-045, and 1613686-047, indicate that at least 50% glycerin may be required for antimicrobial effectiveness.

TABLE 4A
AET measurements for composition 1613686-030

| | Inoculum | 1613686-030 | |
|---|---|---|---|
| Organism | Concentration | Day 14 | Day 28 |
| E. coli | 4.3 × 10⁵/mL | <10/mL | <10/mL |
| S. aureus | 3.2 × 10⁵/mL | <10/mL | <10/mL |
| P. aeruginosa | 2.5 × 10⁵/mL | <10/mL | <10/mL |
| C. albicans | 2.9 × 10⁵/mL | 3.4 × 10²/mL | <10/mL |
| A. brasiliensis | 3.0 × 10⁵/mL | 1.9 × 10⁴/mL | 9.3 × 10³/mL |

TABLE 4B
AET measurements for compositions 1613686-041 and 1613686-042

| | Inoculum | 1613686-041 | | 1613686-042 | |
|---|---|---|---|---|---|
| Organism | Concentration | Day 14 | Day 28 | Day 14 | Day 28 |
| E. coli | 4.5 × 10⁵/mL | <10/mL | <10/mL | <10/mL | <10/mL |
| S. aureus | 3.5 × 10⁵/mL | <10/mL | <10/mL | <10/mL | <10/mL |
| P. aeruginosa | 3.6 × 10⁵/mL | <10/mL | <10/mL | <10/mL | <10/mL |
| C. albicans | 3.6 × 10⁵/mL | TNTC | 3.0 × 10⁵/mL | 4.2 × 10⁵/mL | 2.3 × 10²/mL |
| A. brasiliensis | 2.0 × 10⁵/mL | 7.0 × 10³/mL | 2.2 × 10⁵/mL | TNTC | 2.1 × 10⁵/mL |

TABLE 4C
AET measurements for compositions 1613686-043, 1613686-044, 1613686-045, and 1613686-046

| | Inoculum | 1613686-043 | | 1613686-044 | | 1613686-045 | | 1613686-046 | |
|---|---|---|---|---|---|---|---|---|---|
| Organism | Concentration | Day 14 | Day 28 | Day 14 | Day 28 | Day 14 | Day 28 | Day 14 | Day 28 |
| E. coli | 4.5 × 10⁵/mL | <10/mL | <10/mL | <10/mL | <10/mL | <10/mL | <10/mL | <10/mL | <10/mL |
| S. aureus | 3.5 × 10⁵/mL | <10/mL | <10/mL | <10/mL | <10/mL | <10/mL | <10/mL | <10/mL | <10/mL |
| P. aeruginosa | 3.6 × 10⁵/mL | <10/mL | <10/mL | <10/mL | <10/mL | <10/mL | <10/mL | <10/mL | <10/mL |
| C. albicans | 3.0 × 10⁵/mL | 1.5 × | 90/mL | 4.1 × | 1.1 × | 1.5 × | 6.7 × | <10/mL | <10/mL |
| | | 10³/mL | | 10⁴/mL | 10⁶/mL | 10³/mL | 10⁴/mL | | |
| A. brasiliensis | 2.0 × 10⁵/mL | 2.3 × | 2.5 × | 3.3 × | 4.5 × | 3.7 × | 5.0 × | 1.2 × | 2.0 × |
| | | 10⁵/mL | 10⁵/mL | 10⁵/mL | 10⁵/mL | 10⁵/mL | 10⁵/mL | 10⁵/mL | 10⁵/mL |

TABLE 4D
AET measurements for composition 1613686-049

| | Inoculum | 1613686-049 | |
|---|---|---|---|
| Organism | Concentration | Day 14 | Day 28 |
| E. coli | 3.8 × 10⁵/mL | 4.4 × 10⁶/mL | 9.0 × 10⁵/mL |
| S. aureus | 2.3 × 10⁵/mL | 4.3 × 10⁶/mL | 1.5 × 10⁶/mL |
| P. aeruginosa | 2.5 × 10⁵/mL | 3.0 × 10⁶/mL | 1.8 × 10⁶/mL |
| C. albicans | 3.0 × 10⁵/mL | 1.9 × 10⁶/mL | <10/mL |
| A. brasiliensis | 2.0 × 10⁵/mL | 1.5 × 10⁵/mL | 1.1 × 10⁵/mL |

TABLE 4E
AET measurements for compositions 1613686-047, 1613686-048, and 1613686-050

| | Inoculum | 1613686-047 | | 1613686-048 | | 1613686-050 | |
|---|---|---|---|---|---|---|---|
| Organism | Concentration | Day 14 | Day 28 | Day 14 | Day 28 | Day 14 | Day 28 |
| E. coli | 3.5 × 10⁵/mL | <10/mL | <10/mL | <10/mL | <10/mL | <10/mL | <10/mL |
| S. aureus | 2.4 × 10⁵/mL | <10/mL | <10/mL | <10/mL | <10/mL | <10/mL | <10/mL |
| P. aeruginosa | 2.5 × 10⁵/mL | <10/mL | <10/mL | <10/mL | <10/mL | <10/mL | <10/mL |
| C. albicans | 3.2 × 10⁵/mL | 3.8 × | 3.8 × | 1.4 × | 1.3 × | 6.3 × | 5.3 × |
| | | 10⁴/mL | 10³/mL | 10⁴/mL | 10⁴/mL | 10³/mL | 10³/mL |
| A. brasiliensis | 2.0 × 10⁵/mL | 1.6 × | 2.2 × | 5.6 × | 5.1 × | 4.4 × | 4.6 × |
| | | 10⁵/mL | 10⁵/mL | 10⁴/mL | 10⁴/mL | 10⁴/mL | 10⁴/mL |

TABLE 4F
AET measurements for compositions 1613686-051, 1613686-052, and 1613686-053

| | Inoculum | 1613686-051 | | 1613686-052 | | 1613686-053 | |
|---|---|---|---|---|---|---|---|
| Organism | Concentration | Day 14 | Day 28 | Day 14 | Day 28 | Day 14 | Day 28 |
| E. coli | 3.5 × 10⁵/mL | <10/mL | <10/mL | <10/mL | <10/mL | <10/mL | <10/mL |
| S. aureus | 2.4 × 10⁵/mL | <10/mL | <10/mL | <10/mL | <10/mL | <10/mL | <10/mL |
| P. aeruginosa | 2.5 × 10⁵/mL | <10/mL | <10/mL | <10/mL | <10/mL | <10/mL | <10/mL |
| C. albicans | 3.2 × 10⁵/mL | 8.8 × | 8.4 × | 1.6 × | 8.1 × | 9.5 × | 1.1 × |
| | | 10⁵/mL | 10⁵/mL | 10⁴/mL | 10³/mL | 10⁵/mL | 10⁶/mL |
| A. brasiliensis | 2.0 × 10⁵/mL | 1.5 × | 1.3 × | 5.2 × | 5.6 × | 2.3 × | 2.0 × |
| | | 10⁵/mL | 10⁵/mL | 10³/mL | 10³/mL | 10⁵/mL | 10⁵/mL |

Upon completion of AET, each composition was scored for its compliance with the AET criteria (Table 1, Table 2 & Table 3). Surprisingly, the presence of the microbial substrate xanthan gum did not alter the antimicrobial effectiveness of test compositions in which 50% glycerin and 10% sorbitol were present. For example, aqueous-based composition 1613686-048 contained a high level of xanthan gum (0.75%) and remained AET compliant (Table 3). When pH and viscosity were altered, the antimicrobial effectiveness of test compositions in which 50% glycerin and 10% sorbitol were present remained unaltered. For example, aqueous-based composition 1613686-046 had a relatively high pH of 7.6 and a relatively low viscosity of 202.7, and remained AET compliant (Table 2). These results suggest that 50% glycerin and 10% sorbitol are sufficient to maintain the antimicrobial effectiveness of a solution in the presence of microbial substrates such as xanthan gum.

### Example 2

### Aqueous-Based Compositions

The following example describes the composition of aqueous-based compositions.

TABLE 5
Aqueous-based composition containing Dextromethorphan
HBr and Phenylephrine HCl

| Ingredient: | % w/v |
|---|---|
| Dextromethorphan HBr | 0.100 |
| Phenylephrine HCl | 0.050 |
| Citric acid | 0.750 |
| Trisodium citrate | 0.100 |
| Glycerin | 50.000 |
| Sorbitol solution | 10.000 |
| Xanthan gum | ≥0.250 |
| Purified water | q.s. |
| Total | 100.00 |

TABLE 6
Aqueous-based composition containing Phenylephrine
HCl and Chlorpheniramine maleate

| Ingredient: | % w/v |
|---|---|
| Phenylephrine HCl | 0.050 |
| Chlorpheniramine maleate | 0.020 |
| Citric acid | 0.750 |
| Trisodium citrate | 0.100 |
| Glycerin | 50.000 |
| Sorbitol solution | 10.000 |
| Xanthan gum | ≥0.250 |
| Purified water | q.s. |
| Total | 100.00 |

TABLE 7
Aqueous-based composition containing Dextromethorphan
HBr and Chlorpheniramine maleate

| Ingredient: | % w/v |
|---|---|
| Dextromethorphan HBr | 0.100 |
| Chlorpheniramine maleate | 0.020 |
| Citric acid | 0.750 |
| Trisodium citrate | 0.100 |
| Glycerin | 50.000 |
| Sorbitol solution | 10.000 |
| Xanthan gum | ≥0.250 |
| Purified water | q.s. |
| Total | 100.00 |

These compositions will undergo AET testing as described in Example 1.

What is claimed is:

1. An aqueous-based composition, comprising:
one or more natural polymers; and
two or more natural polyols,
wherein the aqueous-based composition does not comprise any pharmaceutical antimicrobial preservatives or any artificial antimicrobial preservatives.

2. An aqueous-based composition, comprising:
one or more natural polymers; and
two or more natural polyols,
wherein the aqueous-based composition does not comprise butylparaben, methylparaben, propylparaben, EDTA, sodium benzoate, sodium propionate, or any combination thereof.

3. (canceled)

4. (canceled)

5. An aqueous-based composition, comprising:
a pharmaceutically effective amount of one or more active pharmaceutical ingredients;
one or more natural polymers; and
two or more natural polyols,
wherein the aqueous-based composition does not comprise any pharmaceutical antimicrobial preservatives.

6. An aqueous-based composition, comprising:
a pharmaceutically effective amount of one or more active pharmaceutical ingredients;
one or more natural polymers; and
two or more natural polyols,
wherein the aqueous-based composition does not comprise butylparaben, methylparaben, propylparaben, EDTA, sodium benzoate, sodium propionate, or any combination thereof.

7. (canceled)

8. (canceled)

9. The aqueous-based composition of claim 1, wherein the one or more natural polymers are one or more polysaccharides.

10. The aqueous-based composition of claim 9, wherein the one or more polysaccharides are selected from the group consisting of xanthan gum, alginate (alginic acid), carageenan, pectin, acacia gum (gum arabic), tragacanth, locust bean gum, guar gum, cellulose gum, gellan gum, chitosan, and any combination thereof.

11.-14. (canceled)

15. The aqueous-based composition of claim 1, wherein the aqueous-based composition comprises the one or more natural polymers in an amount that ranges from 0.5% w/v to 1% w/v.

16. The aqueous-based composition of claim 15, wherein the two or more natural polyols are glycerin and one or more sugar alcohols.

17. The aqueous-based composition of claim 16, wherein the one or more sugar alcohols are selected from the group consisting of sorbitol, mannitol, maltitol, xylitol, erythritol, isomalt or any combination thereof.

18.-55. (canceled)

56. The aqueous-based composition of claim 1, wherein the aqueous-based composition does not comprise an artificial flavorant.

57. (canceled)

58. The aqueous-based composition of claim 1, wherein the aqueous-based composition does not comprise an artificial colorant.

59. (canceled)

60. The aqueous-based composition of claim 1, wherein the aqueous-based composition does not comprise an artificial sweetener.

61. The aqueous-based composition of claim 1, wherein the aqueous-based composition is in the form of a solution, a syrup, a suspension, or a gel.

62. (canceled)

63. The aqueous-based composition of claim 1, wherein the aqueous-based composition is an oral composition, a buccal composition, a sublingual composition, an inhalation composition, a nebulization composition, an ophthalmic composition, an otic composition, a nasal composition, an intravenous composition, an intramuscular composition, an intrathecal composition, an intraosseous composition, a rectal composition, a subcutaneous composition, a cutaneous composition, or a transdermal composition.

64.-96. (canceled)
